SEQUENCE LISTING (32 sequences)

SEQ ID NO: 1 - protein, 296 aa, Homo sapiens
MMLPLQGAQMLQMLEKSLRKSLPASLKVYGTVFHINHGNPFNLKAVVDKWPDFNTVVVCPQEQDMTDDLDHYTNTYQIYSKDPQNCQEFLGSPELINWKQHLQIQSSQPSLNEAIQNLAAIKSFKVKQTQRILYMAAETAKELTPFLLKSKILSPNGGKPKAINQEMFKLSSMDVTHAHLVNKFWHFGGNERSQRFIERCIQTFPTCCLLGPEGTPVCWDLMDQTGEMRMAGTLPEYRLHGLVTYVIYSHAQKLGKLGFPVYSHVDYSNEAMQKMSYTLQHVPIPRSWNQWNCVPL

SEQ ID NO: 2 - DNA, 891 bp, Homo sapiens
atgatgttaccattgcaaggtgcccagatgctgcagatgctggagaaatccttgaggaagagcctcccagcatccttaaaggtttatggaactgtctttcacataaaccacggaaatccattcaatctgaaggctgtggtggacaagtggcctgattttaatacagtggttgtctgccctcaggagcaggatatgacagatgaccttgatcactataccaatacttaccaaatctactccaaagatccccaaaactgtcaggaattccttggatcaccagaactcatcaactggaaacagcatttacagattcaaagttcacagcctagcctgaatgaggctatacaaaatcttgcagccattaagtccttcaaagtcaaacaaacacaacgcattctctatatggcagctgaaacagccaaggaactgactcctttcctgctgaaatcaaagattttatctcccaatggtggcaaacccaaggccatcaaccaagagatgtttaaactctcatctatggatgttacccatgctcacttggtgaataaattctggcattttggtggtaatgagaggagccagagattcattgagcgctgcattcagacctttcccacctgctgtctcctggggcctgaggggacccctgtgtgctgggatctaatggaccagactggagagatgagaatggcaggcaccttgccggaataccggctccacggccttgtgacgtatgtcatctattcccacgcccagaaattgggcaaacttgggtttcctgtctattctcatgtagactacagcaatgaagctatgcaaaaaatgagttacacactgcaacatgttcccattcccagaagctggaaccagtggaactgtgtacctctgtga

SEQ ID NO: 3 - protein, 302 aa, Homo sapiens
MILLNNSHKLLALYKSLARSIPESLKVYGSVYHINHGNPFNMEVLVDSWPEYQMVIIRPQKQEMTDDMDSYTNVYRMFSKEPQKSEEVLKNCEIVNWKQRLQIQGLQESLGEGIRVATFSKSVKVEHSRALLLVTEDILKLNASSKSKLGSWAETGHPDDEFESETPNFKYAQLDVSYSGLVNDNWKRGKNERSLHYIKRCIEDLPAACMLGPEGVPVSWVTMDPSCEVGMAYSMEKYRRTGNMARVMVRYMKYLRQKNIPFYISVLEENEDSRRFVGQFGFFEASCEWHQWTCYPQNLVPF

SEQ ID NO: 4 - DNA, 909 bp, Homo sapiens
atgatcctactgaataactcccataagctgctggccctatacaaatccttggccaggagcatccctgagtccctgaaggtgtatggctctgtgtatcacatcaatcacgggaaccccttcaacatggaggtgctggtggattcctggcctgaatatcagatggttattatccggcctcaaaagcaggagatgactgatgacatggattcatacacaaacgtatatcgtatgttctccaaagagcctcaaaaatcagaagaagttttgaaaaattgtgagatcgtaaactggaaacagagactccaaatccaaggtcttcaagaaagtttaggtgaggggataagagtggctacattttcaaagtcagtgaaagtagagcattcgagagcactcctcttggttacggaagatattctgaagctcaatgcctccagtaaaagcaagcttggaagctgggctgagacaggccacccagatgatgaatttgaaagtgaaactcccaactttaagtatgcccagctggatgtctcttattctgggctggtaaatgacaactggaagcgagggaagaatgagaggagcctgcattacatcaagcgctgcatagaagacctgccagcagcctgtatgctcggcccagagggagtcccggtctcatgggtaaccatggacccttcttgtgaagtaggaatggcctacagcatggaaaaataccgaaggacaggcaacatggcacgagtgatggtgcgatacatgaaatatctgcgtcagaagaatattccattttacatctctgtgttggaagaaaatgaagactcccgcagatttgtggggcagtttggtttctttgaggcctcctgtgagtggcaccaatggacttgctacccacagaatctagttccattttag

SEQ ID NO: 5 - protein, 294 aa, Homo sapiens
MLVLHNSQKLQILYKSLEKSIPESIKVYGAIFNIKDKNPFNMEVLVDAWPDYQIVITRPQKQEMKDDQDHYTNTYHIFTKAPDKLEEVLSYSNVISWEQTLQIQGCQEGLDEAIRKVATSKSVQVDYMKTILFIPELPKKHKTSSNDKMELFEVDDDNKEGNFSNMFLDASHAGLVNEHWAFGKNERSLKYIERCLQDFLGFGVLGPEGQLVSWIVMEQSCELRMGYTVPKYRHQGNMLQIGYHLEKYLSQKEIPFYFHVADNNEKSLQALNNLGFKICPCGWHQWKCTPKKYC

SEQ ID NO: 6 - DNA, 885 bp, Homo sapiens
atgcttgtgcttcataactctcagaagctgcagattctgtataaatccttagaaaagagcatccctgaatccataaaggtatatggcgccattttcaacataaaagataaaaaccctttcaacatggaggtgctggtagatgcctggccagattaccagatcgtcattacccggcctcagaaacaggagatgaaagatgaccaggatcattataccaacacttaccacatcttcaccaaagctcctgacaaattagaggaagtcctgtcatactccaatgtaatcagctgggagcaaactttgcagatccaaggttgccaagagggcttggatgaagcaataagaaaggttgcaacttcaaaatcagtgcaggtagattacatgaaaaccatcctctttataccggaattaccaaagaaacacaagacctcaagtaatgacaagatggagttatttgaagtggatgatgataacaaggaaggaaacttttcaaacatgttcttagatgcttcacatgcaggtcttgtgaatgaacactgggcctttgggaaaaatgagaggagcttgaaatatattgaacgctgcctccaggattttctaggatttggtgtgctgggtccagagggccagcttgtctcttggattgtgatggaacagtcctgtgagttgagaatgggttatactgtccccaaatacagacaccaaggcaacatgttgcaaattggttatcatcttgaaaagtatctttctcagaaagaaatcccattttatttccatgtggcagataataatgagaaaagcctacaggcactgaacaatttggggtttaagatttgtccttgtggctggcatcagtggaaatgcacccccaagaaatattgttga

SEQ ID NO: 7 - protein, 288 aa, Homo sapiens
MLVLNCSTKLLILEKMLKSCFPESLKVYGAVMNINRGNPFQKEVVLDSWPDFKAVITRRQREAETDNLDHYTNAYAVFYKDVRAYRQLLEECDVFNWDQVFQIQGLQSELYDVSKAVANSKQLNIKLTSFKAVHFSPVSSLPDTSFLKGPSPRLTYLSVANADLLNRTWSRGGNEQCLRYIANLISCFPSVCVRDEKGNPVSWSITDQFATMCHGYTLPEHRRKGYSRLVALTLARKLQSRGFPSQGNVLDDNTASISLLKSLHAEFLPCRFHRLILTPATFSGLPHL

SEQ ID NO: 8 - DNA, 2130 bp, Homo sapiens
aagaataaacttaccatttatataaaagggctactggactgatacacagctgaaaaccctcagttctggactgaactcccagcaggtgtggagttgcaagagctctggaaaagatgttggtgctaaactgttctaccaaattactgatactggagaaaatgttgaagagttgctttcctgaatcactcaaggtttacggagcggtgatgaacataaatcgtgggaacccctttcaaaaggaagtggtgttggattcatggccggatttcaaagctgttatcacccgacgacaaagagaggctgagacagataaccttgatcattatactaatgcctatgctgtgttctacaaggatgtcagggcttatcgacagctattggaagaatgtgatgtttttaactgggaccaagtttttcaaatacaagggctgcagagtgagttatatgatgtttccaaagcggttgccaattcaaagcagttgaatataaagctaacttccttcaaggctgttcatttttctcctgtttcatctctgccagataccagtttcctcaaggggccttccccacgactaacctacctgagtgttgccaatgcggatctactcaaccggacttggtcccggggaggcaatgaacaatgtctccggtacatcgccaacctcatctcctgcttccctagtgtgtgtgtccgggatgagaagggaaacccggtctcctggtccatcacagaccagtttgccaccatgtgccatggctacaccctgccagaacatcgcaggaaaggttacagccggctggtggccctcacgctggccaggaagttgcaaagccggggattcccctctcaggggaacgtcctggatgacaacacggcgtctataagcctcctgaagagtctccatgctgagttcttgccttgtcgcttccacaggcttattctcacccctgcgactttctctggcctgcctcacctctagcccagtaaaaaactgcagtggttttattactttccctgagcatacacacactcttggctgccaacgaggggagagttaaaatgggaatcaggggactcttgagttgttggaaagggtctggagaatatatacaggatccacttgagaagccttaatttttcgtatctcaggtttctccagtaaatagctgtgggggtgaagagtagctgtggctgaagactgaggacgattgtcctcctgtaggatccactgtaggagaataggttctaaagccagcagttttagtgtactaggagaaattactgcatgagaacaaatgatttaacagaggaccacgtggctactgctttttgattgctgcttggacctctgctctgtattcttaaagccacaccgcttccctactgccatcatattcccctgtccccactgctatgtctcatcaacctctgttcctaacacctctgccaccaagttctctgtagagtaacctcctttttcccctttaattacttgctctttacttctgcctaggactctagcctatagttcactgccctgggaatgttcaaatatagtggttcttacattttagtgtttatcagaatcacccagagggcaggttgcaacacacatcactaggcctctccttctacgaggtagggcccaaaatttgcatttctaacagcttcccactgcttatttgccttggatgaatgacaatatgggcattttgatgctataaacaaatgctgtcaccatagaactagactttacctataacctatttcagcccccttatttatagtctactttcccatataaaactaagatttatatataggggtgtttgggggtatgcaaatgaatatataacatatatgcatacacatatatatacattctcttcatttcttttatatgtataggtatatactcatagaattttgataagataataaattttaaccctttgattacatatgaaaaatttgaggaccagagaaaataaatgactttttcaagattatattctttataatcagtactggaggcaaagccagaatgctgccattttaattccaatctgttattttcactaaatcatgtatccttttttataatgaaaattaaaatgcttacataatta

SEQ ID NO: 9 - protein, 19 aa, Homo sapiens
  MOD_RES (2)..(2): Ala or Glu
PXSXKVYGXXXXIXXXNPF

SEQ ID NO: 10 - protein, 10 aa, Homo sapiens
  MOD_RES (2)..(2): Asp or Asn
DXXDXYTNXY

SEQ ID NO: 11 - protein, 8 aa, Homo sapiens
  MOD_RES (2)..(2): Lys, Asp or Glu
WXQXXQIQ

SEQ ID NO: 12 - protein, 12 aa, Homo sapiens
  MOD_RES (2)..(2): Val or Leu
LXNXXWXXGXNE

SEQ ID NO: 13 - protein, 9 aa, Homo sapiens
  MOD_RES (1)..(1): Gly or Asp
XXXGXXVXW

SEQ ID NO: 14 - DNA, 891 bp, Artificial Sequence
  Description of Artificial Sequence Synthetic polynucleotide
atgatgctgccgctgcagggcgcacagatgctgcaaatgctggagaagtccctgcgtaagagcttgccggcttccctgaaagtttacggtaccgtgttccacattaatcacggcaacccatttaacctgaaagccgtggttgacaagtggcctgactttaacactgtggttgtgtgcccgcaagagcaagacatgaccgacgatctggatcattatacgaatacgtatcagatctatagcaaagacccgcaaaattgccaggaatttctgggtagcccggagttgatcaattggaaacagcatctgcagattcaaagcagccaaccgagcttgaacgaagcgatccagaacctggcagcgattaagtcgttcaaggtcaagcagacccaacgcattttgtacatggctgccgaaaccgcgaaagaactgacgccgttcctgttgaaaagcaagatcctgtccccgaatggtggcaagccgaaagcgatcaatcaagaaatgttcaaactgagcagcatggatgtcacccacgcgcacctggtcaacaaattctggcacttcggcggcaacgagcgtagccaacgttttatcgagcgctgtattcagacgtttccgacctgttgtctgctgggtcctgagggtactccggtgtgctgggatctgatggatcagaccggtgagatgcgtatggccggtaccctgccagagtatcgcctgcacggcctggtcacgtacgttatctacagccatgcgcagaaactgggtaagctgggtttcccggtgtactctcatgtcgactacagcaatgaagcaatgcaaaagatgagctataccctgcagcacgttccgattccgcgttcttggaatcagtggaactgcgttccgctgtaa

SEQ ID NO: 15 - DNA, 885 bp, Artificial Sequence
  Description of Artificial Sequence Synthetic polynucleotide
atgctggtgctgcataattcgcaaaagctgcaaatcctgtacaaaagcctggagaagtccattccggagagcattaaagtgtatggtgcgatctttaacattaaggacaaaaaccctttcaacatggaagttctggttgacgcgtggccggattatcagatcgttattacccgtccacagaagcaagagatgaaagacgatcaagatcactacacgaatacctaccacatctttacgaaggctccggacaagctggaagaagtgttgagctattctaacgttatcagctgggagcaaacgctgcagattcagggttgtcaagagggcctggacgaagccatccgcaaagtcgcgaccagcaaaagcgtccaagttgattacatgaaaaccatcctgttcatcccggaattgccgaagaaacataagacttccagcaacgataagatggaactgttcgaggtcgatgacgacaataaggaaggcaactttagcaacatgtttttggatgcatctcatgccggtctggtgaacgagcactgggcgttcggcaaaaatgaacgtagcctgaaatacattgagcgttgcctgcaggacttcctgggctttggtgtcctgggtccggaaggtcaactggtgagctggattgtgatggagcagagctgcgagttgcgtatgggctataccgtcccgaagtaccgccaccagggtaatatgctgcagatcggttatcatctggagaaatatctgagccagaaagaaattccgttttacttccacgttgcggacaataatgagaaaagcctgcaagcactgaacaatctgggtttcaagatttgcccgtgtggctggcaccagtggaaatgtaccccgaagaagtactgctaa

SEQ ID NO: 16 - DNA, 282 bp, Artificial Sequence
  Description of Artificial Sequence Synthetic polynucleotide
tacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaatt

SEQ ID NO: 17 - DNA, 21 bp, Artificial Sequence
  Description of Artificial Sequence Synthetic oligonucleotide
aaagcaaggaggagcagacgt

SEQ ID NO: 18 - DNA, 65 bp, Artificial Sequence
  Description of Artificial Sequence Synthetic oligonucleotide
agccgccccgcagggcgctccgcaggccgcttccggaccactccggaagcggccgtgcggtcgga

SEQ ID NO: 19 - DNA, 1277 bp, Artificial Sequence
  Description of Artificial Sequence Synthetic polynucleotide
ggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtatgatgttaccattgcaaggtgcccagatgctgcagatgctggagaaatccttgaggaagagcctcccagcatccttaaaggtttatggaactgtctttcacataaaccacggaaatccattcaatctgaaggctgtggtggacaagtggcctgattttaatacagtggttgtctgccctcaggagcaggatatgacagatgaccttgatcactataccaatacttaccaaatctactccaaagatccccaaaactgtcaggaattccttggatcaccagaactcatcaactggaaacagcatttacagattcaaagttcacagcctagcctgaatgaggctatacaaaatcttgcagccattaagtccttcaaagtcaaacaaacacaacgcattctctatatggcagctgaaacagccaaggaactgactcctttcctgctgaaatcaaagattttatctcccaatggtggcaaacccaaggccatcaaccaagagatgtttaaactctcatctatggatgttacccatgctcacttggtgaataaattctggcattttggtggtaatgagaggagccagagattcattgagcgctgcattcagacctttcccacctgctgtctcctggggcctgaggggacccctgtgtgctgggatctaatggaccagactggagagatgagaatggcaggcaccttgccggaataccggctccacggccttgtgacgtatgtcatctattcccacgcccagaaattgggcaaacttgggtttcctgtctattctcatgtagactacagcaatgaagctatgcaaaaaatgagttacacactgcaacatgttcccattcccagaagctggaaccagtggaactgtgtacctctgtgaagccgccccgcagggcgctccgcaggccgcttccggaccactccggaagcggccgtgcggtcggaaagctttctaga

SEQ ID NO: 20 - DNA, 1663 bp, Artificial Sequence
  Description of Artificial Sequence Synthetic polynucleotide
ggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtatgatgctgccgctgcagggcgcacagatgctgcaaatgctggagaagtccctgcgtaagagcttgccggcttccctgaaagtttacggtaccgtgttccacattaatcacggcaacccatttaacctgaaagccgtggttgacaagtggcctgactttaacactgtggttgtgtgcccgcaagagcaagacatgaccgacgatctggatcattatacgaatacgtatcagatctatagcaaagacccgcaaaattgccaggaatttctgggtagcccggagttgatcaattggaaacagcatctgcagattcaaagcagccaaccgagcttgaacgaagcgatccagaacctggcagcgattaagtcgttcaaggtcaagcagacccaacgcattttgtacatggctgccgaaaccgcgaaagaactgacgccgttcctgttgaaaagcaagatcctgtccccgaatggtggcaagccgaaagcgatcaatcaagaaatgttcaaactgagcagcatggatgtcacccacgcgcacctggtcaacaaattctggcacttcggcggcaacgagcgtagccaacgttttatcgagcgctgtattcagacgtttccgacctgttgtctgctgggtcctgagggtactccggtgtgctgggatctgatggatcagaccggtgagatgcgtatggccggtaccctgccagagtatcgcctgcacggcctggtcacgtacgttatctacagccatgcgcagaaactgggtaagctgggtttcccggtgtactctcatgtcgactacagcaatgaagcaatgcaaaagatgagctataccctgcagcacgttccgattccgcgttcttggaatcagtggaactgcgttccgctgtaaagccgccccgcagggcgctccgcaggccgcttccggaccactccggaagcggccgtgcggtcggaaagctttctagaagccgccccgcagggcgctccgcaggccgcttccggaccactccggaagcggccgtgcggtcggaaagctttctaga

SEQ ID NO: 21 - DNA, 1271 bp, Artificial Sequence
  Description of Artificial Sequence Synthetic polynucleotide
ggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtatgcttgtgcttcataactctcagaagctgcagattctgtataaatccttagaaaagagcatccctgaatccataaaggtatatggcgccattttcaacataaaagataaaaaccctttcaacatggaggtgctggtagatgcctggccagattaccagatcgtcattacccggcctcagaaacaggagatgaaagatgaccaggatcattataccaacacttaccacatcttcaccaaagctcctgacaaattagaggaagtcctgtcatactccaatgtaatcagctgggagcaaactttgcagatccaaggttgccaagagggcttggatgaagcaataagaaaggttgcaacttcaaaatcagtgcaggtagattacatgaaaaccatcctctttataccggaattaccaaagaaacacaagacctcaagtaatgacaagatggagttatttgaagtggatgatgataacaaggaaggaaacttttcaaacatgttcttagatgcttcacatgcaggtcttgtgaatgaacactgggcctttgggaaaaatgagaggagcttgaaatatattgaacgctgcctccaggattttctaggatttggtgtgctgggtccagagggccagcttgtctcttggattgtgatggaacagtcctgtgagttgagaatgggttatactgtccccaaatacagacaccaaggcaacatgttgcaaattggttatcatcttgaaaagtatctttctcagaaagaaatcccattttatttccatgtggcagataataatgagaaaagcctacaggcactgaacaatttggggtttaagatttgtccttgtggctggcatcagtggaaatgcacccccaagaaatattgttgaagccgccccgcagggcgctccgcaggccgcttccggaccactccggaagcggccgtgcggtcggaaagctttctaga

SEQ ID NO: 22 - DNA, 1657 bp, Artificial Sequence
  Description of Artificial Sequence Synthetic polynucleotide
ggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtggatcctacacagcccagtccagactattcggcactgaaattatgggtgaagtggtcaagacctcactaggcaccttaaaaatagcgcaccctgaagaagatttatttgaggtagcccttgcctacctagcttccaagaaagatatcctaacagcacaagagcggaaagatgttttgttctacatccagaacaacctctgctaaaattcctgaaaaattttgcaaaaagttgttgactttatctacaaggtgtggcataatgtgtggaattgtgagcggataacaattaaagcaaggaggagcagacgtatgctggtgctgcataattcgcaaaagctgcaaatcctgtacaaaagcctggagaagtccattccggagagcattaaagtgtatggtgcgatctttaacattaaggacaaaaaccctttcaacatggaagttctggttgacgcgtggccggattatcagatcgttattacccgtccacagaagcaagagatgaaagacgatcaagatcactacacgaatacctaccacatctttacgaaggctccggacaagctggaagaagtgttgagctattctaacgttatcagctgggagcaaacgctgcagattcagggttgtcaagagggcctggacgaagccatccgcaaagtcgcgaccagcaaaagcgtccaagttgattacatgaaaaccatcctgttcatcccggaattgccgaagaaacataagacttccagcaacgataagatggaactgttcgaggtcgatgacgacaataaggaaggcaactttagcaacatgtttttggatgcatctcatgccggtctggtgaacgagcactgggcgttcggcaaaaatgaacgtagcctgaaatacattgagcgttgcctgcaggacttcctgggctttggtgtcctgggtccggaaggtcaactggtgagctggattgtgatggagcagagctgcgagttgcgtatgggctataccgtcccgaagtaccgccaccagggtaatatgctgcagatcggttatcatctggagaaatatctgagccagaaagaaattccgttttacttccacgttgcggacaataatgagaaaagcctgcaagcactgaacaatctgggtttcaagatttgcccgtgtggctggcaccagtggaaatgtaccccgaagaagtactgctaaagccgccccgcagggcgctccgcaggccgcttccggaccactccggaagcggccgtgcggtcggaaagctttctagaagccgccccgcagggcgctccgcaggccgcttccggaccactccggaagcggccgtgcggtcggaaagctttctaga

SEQ ID NO: 23 - DNA, 22 bp, Artificial Sequence
  Description of Artificial Sequence Synthetic primer
ttctgtttctgcttcggtatgt

SEQ ID NO: 24 - DNA, 22 bp, Artificial Sequence
  Description of Artificial Sequence Synthetic primer
gaggcttacttgtctgctttct

SEQ ID NO: 25 - DNA, 22 bp, Artificial Sequence
  Description of Artificial Sequence Synthetic primer
ttctgtttctgcttcggtatgt

SEQ ID NO: 26 - DNA, 22 bp, Artificial Sequence
  Description of Artificial Sequence Synthetic primer
gaggcttacttgtctgctttct

SEQ ID NO: 27 - protein, 316 aa, Artificial Sequence
  Description of Artificial Sequence Synthetic polypeptide
MGSSHHHHHHSSGLVPRGSHGMLPLQGAQMLQMLEKSLRKSLPASLKVYGTVFHINHGNPFNLKAVVDKWPDFNTVVVCPQEQDMTDDLDHYTNTYQIYSKDPQNCQEFLGSPELINWKQHLQIQSSQPSLNEAIQNLAAIKSFKVKQTQRILYMAAETAKELTPFLLKSKILSPNGGKPKAINQEMFKLSSMDVTHAHLVNKFWHFGGNERSQRFIERCIQTFPTCCLLGPEGTPVCWDLMDQTGEMRMAGTLPEYRLHGLVTYVIYSHAQKLGKLGFPVYSHVDYSNEAMQKMSYTLQHVPIPRSWNQWNCVPL

SEQ ID NO: 28 - DNA, 951 bp, Artificial Sequence
  Description of Artificial Sequence Synthetic polynucleotide
atgggcagcagccatcatcatcatcatcacagcagcggcctggtgccgcgcggcagccacggcatgttaccattgcaaggtgcccagatgctgcagatgctggagaaatccttgaggaagagcctcccagcatccttaaaggtttatggaactgtctttcacataaaccacggaaatccattcaatctgaaggctgtggtggacaagtggcctgattttaatacagtggttgtctgccctcaggagcaggatatgacagatgaccttgatcactataccaatacttaccaaatctactccaaagatccccaaaactgtcaggaattccttggatcaccagaactcatcaactggaaacagcatttacagattcaaagttcacagcctagcctgaatgaggctatacaaaatcttgcagccattaagtccttcaaagtcaaacaaacacaacgcattctctatatggcagctgaaacagccaaggaactgactcctttcctgctgaaatcaaagattttatctcccaatggtggcaaacccaaggccatcaaccaagagatgtttaaactctcatctatggatgttacccatgctcacttggtgaataaattctggcattttggtggtaatgagaggagccagagattcattgagcgctgcattcagacctttcccacctgctgtctcctggggcctgaggggacccctgtgtgctgggatctaatggaccagactggagagatgagaatggcaggcaccttgccggaataccggctccacggccttgtgacgtatgtcatctattcccacgcccagaaattgggcaaacttgggtttcctgtctattctcatgtagactacagcaatgaagctatgcaaaaaatgagttacacactgcaacatgttcccattcccagaagctggaaccagtggaactgtgtacctctgtga

SEQ ID NO: 29 - protein, 315 aa, Artificial Sequence
  Description of Artificial Sequence Synthetic polypeptide
MGSSHHHHHHSSGLVPRGSHGMLVLHNSQKLQILYKSLEKSIPESIKVYGAIFNIKDKNPFNMEVLVDAWPDYQIVITRPQKQEMKDDQDHYTNTYHIFTKAPDKLEEVLSYSNVISWEQTLQIQGCQEGLDEAIRKVATSKSVQVDYMKTILFIPELPKKHKTSSNDKMELFEVDDDNKEGNFSNMFLDASHAGLVNEHWAFGKNERSLKYIERCLQDFLGFGVLGPEGQLVSWIVMEQSCELRMGYTVPKYRHQGNMLQIGYHLEKYLSQKEIPFYFHVADNNEKSLQALNNLGFKICPCGWHQWKCTPKKYC

SEQ ID NO: 30 - DNA, 948 bp, Artificial Sequence
  Description of Artificial Sequence Synthetic polynucleotide
atgggcagcagccatcatcatcatcatcacagcagcggcctggtgccgcgcggcagccacggcatgcttgtgcttcataactctcagaagctgcagattctgtataaatccttagaaaagagcatccctgaatccataaaggtatatggcgccattttcaacataaaagataaaaaccctttcaacatggaggtgctggtagatgcctggccagattaccagatcgtcattacccggcctcagaaacaggagatgaaagatgaccaggatcattataccaacacttaccacatcttcaccaaagctcctgacaaattagaggaagtcctgtcatactccaatgtaatcagctgggagcaaactttgcagatccaaggttgccaagagggcttggatgaagcaataagaaaggttgcaacttcaaaatcagtgcaggtagattacatgaaaaccatcctctttataccggaattaccaaagaaacacaagacctcaagtaatgacaagatggagttatttgaagtggatgatgataacaaggaaggaaacttttcaaacatgttcttagatgcttcacatgcaggtcttgtgaatgaacactgggcctttgggaaaaatgagaggagcttgaaatatattgaacgctgcctccaggattttctaggatttggtgtgctgggtccagagggccagcttgtctcttggattgtgatggaacagtcctgtgagttgagaatgggttatactgtccccaaatacagacaccaaggcaacatgttgcaaattggttatcatcttgaaaagtatctttctcagaaagaaatcccattttatttccatgtggcagataataatgagaaaagcctacaggcactgaacaatttggggtttaagatttgtccttgtggctggcatcagtggaaatgcacccccaagaaatattgttga

SEQ ID NO: 31 - DNA, 6100 bp, Artificial Sequence
  Description of Artificial Sequence Synthetic polynucleotide
cctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccacggcatgttaccattgcaaggtgcccagatgctgcagatgctggagaaatccttgaggaagagcctcccagcatccttaaaggtttatggaactgtctttcacataaaccacggaaatccattcaatctgaaggctgtggtggacaagtggcctgattttaatacagtggttgtctgccctcaggagcaggatatgacagatgaccttgatcactataccaatacttaccaaatctactccaaagatccccaaaactgtcaggaattccttggatcaccagaactcatcaactggaaacagcatttacagattcaaagttcacagcctagcctgaatgaggctatacaaaatcttgcagccattaagtccttcaaagtcaaacaaacacaacgcattctctatatggcagctgaaacagccaaggaactgactcctttcctgctgaaatcaaagattttatctcccaatggtggcaaacccaaggccatcaaccaagagatgtttaaactctcatctatggatgttacccatgctcacttggtgaataaattctggcattttggtggtaatgagaggagccagagattcattgagcgctgcattcagacctttcccacctgctgtctcctggggcctgaggggacccctgtgtgctgggatctaatggaccagactggagagatgagaatggcaggcaccttgccggaataccggctccacggccttgtgacgtatgtcatctattcccacgcccagaaattgggcaaacttgggtttcctgtctattctcatgtagactacagcaatgaagctatgcaaaaaatgagttacacactgcaacatgttcccattcccagaagctggaaccagtggaactgtgtacctctgtgataatagggtac

SEQ ID NO: 32 - DNA, 6097 bp, Artificial Sequence
  Description of Artificial Sequence Synthetic polynucleotide
cctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggcagcagccatcatcatcatcatcacagcagcggcctggtgccgcgcggcagccacggcatgcttgtgcttcataactctcagaagctgcagattctgtataaatccttagaaaagagcatccctgaatccataaaggtatatggcgccattttcaacataaaagataaaaaccctttcaacatggaggtgctggtagatgcctggccagattaccagatcgtcattacccggcctcagaaacaggagatgaaagatgaccaggatcattataccaacacttaccacatcttcaccaaagctcctgacaaattagaggaagtcctgtcatactccaatgtaatcagctgggagcaaactttgcagatccaaggttgccaagagggcttggatgaagcaataagaaaggttgcaacttcaaaatcagtgcaggtagattacatgaaaaccatcctctttataccggaattaccaaagaaacacaagacctcaagtaatgacaagatggagttatttgaagtggatgatgataacaaggaaggaaacttttcaaacatgttcttagatgcttcacatgcaggtcttgtgaatgaacactgggcctttgggaaaaatgagaggagcttgaaatatattgaacgctgcctccaggattttctaggatttggtgtgctgggtccagagggccagcttgtctcttggattgtgatggaacagtcctgtgagttgagaatgggttatactgtccccaaatacagacaccaaggcaacatgttgcaaattggttatcatcttgaaaagtatctttctcagaaagaaatcccattttatttccatgtggcagataataatgagaaaagcctacaggcactgaacaatttggggtttaagatttgtccttgtggctggcatcagtggaaatgcacccccaagaaatattgttgataatagggtac

(19) United States
(12) Patent Application Publication
(10) Pub. No.: US 2016/0090577 A1
(43) Pub. Date: Mar. 31, 2016

(54) HETEROLOGOUS EXPRESSION OF GLYCINE N-ACYLTRANSFERASE PROTEINS

(71) Applicant: Dow AgroSciences LLC, Indianapolis, IN (US)

(72) Inventors: Amudhan VENKATESWARAN, Indianapolis, IN (US); Babu RAMAN, Indianapolis, IN (US); Paul SWANSON, Indianapolis, IN (US); Paul LEWER, Indianapolis, IN (US)

(21) Appl. No.: 14/865,724

(22) Filed: Sep. 25, 2015

Related U.S. Application Data
(60) Provisional application No. 62/127,458, filed on Mar. 3, 2015.
(60) Provisional application No. 62/056,197, filed on Sep. 26, 2014.

(51) Int. Cl. C12N 9/10; C12P 13/04

(52) U.S. Cl. CPC C12N 9/1029; C12P 13/04; C12Y 203/01013

(57) ABSTRACT

The present disclosure provides novel compositions and methods for the production and use of polynucleotide sequences encoding a glycine N-acyltransferase protein (GLYAT, GLYATL 1, GLYATL 2, and GLYATL 3) for the biosynthesis of N-acylglycine biosurfactants within a heterologous expression system.

### CROSS-REFERENCE TO RELATED APPLICATIONS

This application claims the benefit under 35 USC §119(e) of U.S. Provisional Application Ser. No. 62/056,197, filed on Sep. 26, 2014, and of U.S. Provisional Application Ser. No. 62/127,458, filed on Mar. 3, 2015, the entire disclosures of both incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: One 57,657 bytes ASCII (Text) file named “14764-241760_SL.txt” created on Sep. 25, 2015.

### BACKGROUND OF THE INVENTION

N-acylglycine surfactants have traditionally been synthesized via chemical manufacturing processes that utilize chemical feedstocks. The production and manufacture of such surfactants rely upon the use of petrochemicals that are a non-renewable energy source. As such, the costs associated with obtaining petrochemical feedstocks fluctuate with the economic markets. N-acylglycine surfactants must be synthesized via complex chemical processes that require numerous steps of distinct and separate chemical reactions. Finally, the traditional manufacturing process of N-acylglycine surfactants produce chemical waste products that must be remediated for proper disposal.

Therefore, a need exists for development of improved synthesis and manufacturing processes of N-acylglycine surfactants acids via renewable production systems such as microbial fermentation.

The foregoing examples of the related art and limitations related therewith are intended to be illustrative and not exclusive. Other limitations of the related art will become apparent to those of skill in the art upon a reading of the specification.

### BRIEF SUMMARY OF THE INVENTION

In an embodiment, the present disclosure is directed to a metabolically-engineered microorganism capable of synthesizing an N-acylglycine biosurfactant, the microorganism comprising a Glycine N-Acyltransferase protein. Generally, the Glycine N-Acyltransferase protein selected from: a polypeptide with at least 90% sequence identity to a GLYAT polypeptide of SEQ ID NO: 1; a polypeptide with at least 90% sequence identity to a GLYATL 1 polypeptide of SEQ ID:3; a polypeptide with at least 90% sequence identity to a GLYATL 2 polypeptide of SEQ ID NO:5; a polypeptide with at least 90% sequence identity to a GLYATL 3 polypeptide of SEQ ID NO:7; a polypeptide comprising at least one of the motifs of: P(A/E)S(L/I)KVYG(T/A/S)(V/I)(F/M/Y)(H/N)I(N/K)(H/R/D)(G/K)NPF (SEQ ID NO: 9), D(D/N)(L/Q/M)D(H/S)YTN(T/A/V)Y (SEQ ID NO: 10), W(K/D/E)Q(H/V/T/R)(L/F)QIQ (SEQ ID NO: 11), L(V/L)N(K/R/E/D)(F/T/H/N)W(H/S/A/K)(F/R)G(G/K)NE (SEQ ID NO: 12), or (G/D)(P/E)(E/K)G(T/N/Q/V)(P/L)V(C/S)W (SEQ ID NO: 13); a variant polypeptide of SEQ ID NO: 1, said variant having Glycine N-Acyltransferase activity and a least 90% sequence identity with a sequence selected from SEQ ID NO: 1; a variant polypeptide of SEQ ID NO:3, said variant having Glycine N-Acyltransferase activity and a least 90% sequence identity with a sequence selected from SEQ ID NO:3; a variant polypeptide of SEQ ID NO:5, said variant having Glycine N-Acyltransferase activity and a least 90% sequence identity with a sequence selected from SEQ ID NO:5; a variant polypeptide of SEQ ID NO:7, said variant having Glycine N-Acyltransferase activity and a least 90% sequence identity with a sequence selected from SEQ ID NO:7; a polypeptide having Glycine N-Acyltransferase activity wherein said polypeptide is encoded by an isolated polynucleotide that hybridizes under stringent conditions with the sense or anti-sense strand of a polynucleotide sequence selected from SEQ ID NO:2, SEQ ID NO:4, SEQ ID NO:6, SEQ ID NO:8, SEQ ID NO:14, or SEQ ID NO:15; and, a polypeptide that facilitates the conversion of acyl-coA and glycine into coA and N-acylglycine and the polypeptide is chosen from a Glycine N-Acyltransferase enzyme of class E.C. 2.3.1.13. In some embodiments, the Glycine N-Acyltransferase protein comprises a polypeptide with at least 90% sequence identity to a GLYAT of SEQ ID NO: 1. In other embodiments, the Glycine N-Acyltransferase protein comprises a polypeptide with at least 90% sequence identity to a GLYATL 1 of SEQ ID NO:3. In further embodiments, the Glycine N-Acyltransferase protein comprises a polypeptide with at least 90% sequence identity to a GLYATL 2 of SEQ ID NO:5. In embodiments, the Glycine N-Acyltransferase protein comprises a polypeptide with at least 90% sequence identity to a GLYATL 3 of SEQ ID NO:7.

In one aspect the microorganism of the subject disclosure is a gram (−) or a gram (+) bacteria. Exemplary gram (+) bacterium can be Bacillus subtilis. Exemplary gram (−) bacteria can be Escherichia coli. In other aspects of the disclosure, a polynucleotide encoding the Glycine N-Acyltransferase protein is expressed by a bacterial promoter. An exemplary bacterial promoter can be a PsPAC bacterial promoter. In another aspect of the disclosure, the polynucleotide encoding the Glycine N-Acyltransferase protein is codon optimized for expression in the microorganism. Exemplary codon optimized polynucleotide encoding the Glycine N-Acyltransferase protein include SEQ ID NO: 14 and SEQ ID NO:15. In a further aspect of the subject disclosure, the polynucleotide encoding the Glycine N-Acyltransferase protein is integrated within a genomic locus of the microorganism. An exemplary genomic locus can be the amyE genomic locus of a microorganism. In an embodiment, the integration within the genomic locus of a microorganism occurs via homologous recombination. In another aspect of the subject disclosure, the polynucleotide encoding the Glycine N-Acyltransferase protein is integrated within an autonomously replicating plasmid. The subject disclosure herein relates to a metabolically-engineered microorganism that expresses a Glycine N-Acyltransferase protein that subsequently results in the synthesis of N-acylglycine from medium chain length β-hydroxy fatty acids.

The present disclosure is further directed to a method for producing N-acylglycine from a microorganism. The microorganism comprising a polynucleotide encoding a Glycine N-Acyltransferase protein is obtained. The microorganism is cultured to produce a medium chain length β-hydroxy fatty acid. The Glycine N-Acyltransferase protein is expressed, wherein the expression of the Glycine N-Acyltransferase protein synthesizes N-acylglycine from the medium chain length β-hydroxy fatty acid. N-acylglycine is purified from the microorganism.

The present disclosure is directed to a method for fermenting N-acylglycine within a microorganism. The microorganism comprising a polynucleotide encoding a Glycine N-Acyltransferase protein is obtained. The Glycine N-Acyltransferase protein is expressed, wherein the expression of the Glycine N-Acyltransferase protein synthesizes N-acylglycine from a medium chain length β-hydroxy fatty acid. N-acylglycine is fermented within the microorganism.

In addition to the exemplary aspects and embodiments described above, further aspects and embodiments will become apparent by study of the following descriptions.

### BRIEF DESCRIPTION OF THE FIGURES

FIG. 1 illustrates a sequence alignment of glycine N-acyltransferase proteins. The structural motifs that are in common between the proteins are identified by underlining.

FIG. 2 illustrates a diagram for the design of a gene construct for expression of glycine N-acyltransferase enzymes in B. subtilis.

FIG. 3 is a summary of structures referred to in Example 9, including isomeric Bacillus products (1 and 2), an analytical standard (3) and products formed in E. coli fermentations (4 through 10).

FIG. 4 illustrates the experimental designs for a shake flask scale fermentation experiment to test the ability of the engineered microbial strains expressing N-acyltransferases to produce N-acylglycine.

FIG. 5 illustrates quantitative LC-SIM-MS results for B. subtilis str. OKB120 engineered strains expressing GLYAT and GLYATL2 enzymes which were used to demonstrate successful production of these novel N-acylglycine compounds, resulting from the integration of the constructs into the genome of B. subtilis str. OKB120 and to quantify the products (1) and (2) of FIG. 3.

### DETAILED DESCRIPTION

### I. Overview

Disclosed herein are Glycine N-Acyltransferase protein sequences for the novel production of N-acylglycine biosurfactants. The Glycine N-Acyltransferase enzymes can selectively bind and condense amino acids to enzymatically enable the in vivo acylation of the amino acid glycine into a medium chain-length β-hydroxy fatty acid peptide chain. As such, the Glycine N-Acyltransferase protein is heterologously expressed in a microorganism species, and subsequently fermented to result in the production of the non-native lipoamino acid, N-acylglycine biosurfactant. Exemplary polypeptides include members of the enzyme class (E.C.) 2.3.1.13. In an embodiment, a polypeptide that facilitates the conversion of acyl-coA (for example, β-hydroxy fatty acid) and glycine into coA and N-acylglycine and the polypeptide is disclosed herein as a Glycine N-Acyltransferase enzyme of E.C. 2.3.1.13.

### II. Terms

Unless defined otherwise, all technical and scientific terms used herein have the same meaning as commonly understood by one of ordinary skill in the art to which this disclosure relates. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference, unless only specific sections of patents or patent publications are indicated to be incorporated by reference.

In order to further clarify this disclosure, the following terms, abbreviations and definitions are provided.

As used herein, the terms “comprises”, “comprising”, “includes”, “including”, “has”, “having”, “contains”, or “containing”, or any other variation thereof, are intended to be non-exclusive or open-ended. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “invention” or “present invention” as used herein is a non-limiting term and is not intended to refer to any single embodiment of the particular invention but encompasses all possible embodiments as disclosed in the application.

As used herein, “endogenous sequence” defines the native form of a polynucleotide, gene or polypeptide in its natural location in the organism or in the genome of an organism.

The term “isolated”, as used herein means having been removed from its natural environment.

The term “purified”, as used herein relates to the isolation of a molecule or compound in a form that is substantially free of contaminants normally associated with the molecule or compound in a native or natural environment and means having been increased in purity as a result of being separated from other components of the original composition. The term “purified nucleic acid” is used herein to describe a nucleic acid sequence which has been separated from other compounds including, but not limited to polypeptides, lipids and carbohydrates.

As used herein, the terms “polynucleotide”, “nucleic acid”, and “nucleic acid molecule” are used interchangeably, and may encompass a singular nucleic acid; plural nucleic acids; a nucleic acid fragment, variant, or derivative thereof; and nucleic acid construct (e.g., messenger RNA (mRNA) and plasmid DNA (pDNA)). A polynucleotide or nucleic acid may contain the nucleotide sequence of a full-length cDNA sequence, or a fragment thereof, including untranslated 5′ and/or 3′ sequences and coding sequence(s). A polynucleotide or nucleic acid may be comprised of any polyribonucleotide or polydeoxyribonucleotide, which may include unmodified ribonucleotides or deoxyribonucleotides or modified ribonucleotides or deoxyribonucleotides. For example, a polynucleotide or nucleic acid may be comprised of single- and double-stranded DNA; DNA that is a mixture of single- and double-stranded regions; single- and double-stranded RNA; and RNA that is mixture of single- and double-stranded regions. Hybrid molecules comprising DNA and RNA may be single-stranded, double-stranded, or a mixture of single- and double-stranded regions. The foregoing terms also include chemically, enzymatically, and metabolically modified forms of a polynucleotide or nucleic acid.

It is understood that a specific DNA or polynucleotide refers also to the complement thereof, the sequence of which is determined according to the rules of deoxyribonucleotide base-pairing. Although only one strand of DNA may be presented in the sequence listings of this disclosure, those having ordinary skill in the art will recognize that the complementary strand can be ascertained and determined from the strand presented herein. Accordingly, a single strand of a polynucleotide can be used to determine the complementary strand, and, accordingly, both strands (i.e., the sense strand and anti-sense strand) are exemplified from a single strand.

As used herein, the term “gene” refers to a nucleic acid that encodes a functional product (RNA or polypeptide/protein). A gene may include regulatory sequences preceding (5′ non-coding sequences) and/or following (3′ non-coding sequences) the sequence encoding the functional product.

As used herein, the term “coding sequence” refers to a nucleic acid sequence that encodes a specific amino acid sequence. A “regulatory sequence” refers to a nucleotide sequence located upstream (e.g., 5′ non-coding sequences), within, or downstream (e.g., 3′ non-coding sequences) of a coding sequence, which influence the transcription, RNA processing or stability, or translation of the associated coding sequence. Regulatory sequences include, for example and without limitation: promoters; translation leader sequences; introns; polyadenylation recognition sequences; RNA processing sites; effector binding sites; and stem-loop structures.

As used herein, the term “polypeptide” includes a singular polypeptide, plural polypeptides, and fragments thereof. This term refers to a molecule comprised of monomers (amino acids) linearly linked by amide bonds (also known as peptide bonds). The term “polypeptide” refers to any chain or chains of two or more amino acids, and does not refer to a specific length or size of the product. Accordingly, peptides, dipeptides, tripeptides, oligopeptides, protein, amino acid chain, and any other term used to refer to a chain or chains of two or more amino acids, are included within the definition of“polypeptide”, and the foregoing terms are used interchangeably with “polypeptide” herein. A polypeptide may be isolated from a natural biological source or produced by recombinant technology, but a specific polypeptide is not necessarily translated from a specific nucleic acid. A polypeptide may be generated in any appropriate manner, including for example and without limitation, by chemical synthesis. Likewise, a polypeptide may be generated by expressing a native coding sequence, or portion thereof, that are introduced into an organism in a form that is different from the corresponding native coding sequence.

In contrast, the term “heterologous” refers to a polynucleotide, gene or polypeptide that is not normally found at its location in the reference (host) organism. For example, a heterologous nucleic acid may be a nucleic acid that is normally found in the reference organism at a different genomic location. By way of further example, a heterologous nucleic acid may be a nucleic acid that is not normally found in the reference organism. A host organism comprising a hetereologous polynucleotide, gene or polypeptide may be produced by introducing the heterologous polynucleotide, gene or polypeptide into the host organism. In particular examples, a heterologous polynucleotide comprises a native coding sequence, or portion thereof, that is reintroduced into a source organism in a form that is different from the corresponding native polynucleotide. In particular examples, a heterologous gene comprises a native coding sequence, or portion thereof, that is reintroduced into a source organism in a form that is different from the corresponding native gene. For example, a heterologous gene may include a native coding sequence that is a portion of a chimeric gene including non-native regulatory regions that is reintroduced into the native host. In particular examples, a heterologous polypeptide is a native polypeptide that is reintroduced into a source organism in a form that is different from the corresponding native polypeptide.

A heterologous gene or polypeptide may be a gene or polypeptide that comprises a functional polypeptide or nucleic acid sequence encoding a functional polypeptide that is fused to another gene or polypeptide to produce a chimeric or fusion polypeptide, or a gene encoding the same. Genes and proteins of particular embodiments include specifically exemplified full-length sequences and portions, segments, fragments (including contiguous fragments and internal and/or terminal deletions compared to the full-length molecules), variants, mutants, chimerics, and fusions of these sequences.

As used herein, the term “modification” can refer to a change in a polynucleotide disclosed herein that results in reduced, substantially eliminated or eliminated activity of a polypeptide encoded by the polynucleotide, as well as a change in a polypeptide disclosed herein that results in reduced, substantially eliminated or eliminated activity of the polypeptide. Alternatively, the term “modification” can refer to a change in a polynucleotide disclosed herein that results in increased or enhanced activity of a polypeptide encoded by the polynucleotide, as well as a change in a polypeptide disclosed herein that results in increased or enhanced activity of the polypeptide. Such changes can be made by methods well known in the art, including, but not limited to, deleting, mutating (e.g., spontaneous mutagenesis, random mutagenesis, mutagenesis caused by mutator genes, or transposon mutagenesis), substituting, inserting, down-regulating, altering the cellular location, altering the state of the polynucleotide or polypeptide (e.g., methylation, phosphorylation or ubiquitination), removing a cofactor, introduction of an antisense RNA/DNA, introduction of an interfering RNA/DNA, chemical modification, covalent modification, irradiation with UV or X-rays, homologous recombination, mitotic recombination, promoter replacement methods, and/or combinations thereof. Guidance in determining which nucleotides or amino acid residues can be modified, can be found by comparing the sequence of the particular polynucleotide or polypeptide with that of homologous polynucleotides or polypeptides, e.g., yeast or bacterial, and maximizing the number of modifications made in regions of high homology (conserved regions) or consensus sequences.

The term “derivative”, as used herein, refers to a modification of a sequence set forth in the present disclosure. Illustrative of such modifications would be the substitution, insertion, and/or deletion of one or more bases relating to a nucleic acid sequence of a coding sequence disclosed herein that preserve, slightly alter, or increase the function of a coding sequence disclosed herein in crop species. Such derivatives can be readily determined by one skilled in the art, for example, using computer modeling techniques for predicting and optimizing sequence structure. The term “derivative” thus also includes nucleic acid sequences having substantial sequence identity with the disclosed coding sequences herein such that they are able to have the disclosed functionalities for use in producing embodiments of the present disclosure.

The term “promoter” refers to a DNA sequence capable of controlling the expression of a nucleic acid coding sequence or functional RNA. In examples, the controlled coding sequence is located 3′ to a promoter sequence. A promoter may be derived in its entirety from a native gene, a promoter may be comprised of different elements derived from different promoters found in nature, or a promoter may even comprise rationally designed DNA segments. It is understood by those skilled in the art that different promoters can direct the expression of a gene in different cell types, or at different stages of development, or in response to different environmental or physiological conditions. Examples of all of the foregoing promoters are known and used in the art to control the expression of heterologous nucleic acids. Promoters that direct the expression of a gene in most cell types at most times are commonly referred to as “constitutive promoters.” Furthermore, while those in the art have (in many cases unsuccessfully) attempted to delineate the exact boundaries of regulatory sequences, it has come to be understood that DNA fragments of different lengths may have identical promoter activity. The promoter activity of a particular nucleic acid may be assayed using techniques familiar to those in the art.

The term “operably linked” refers to an association of nucleic acid sequences on a single nucleic acid, wherein the function of one of the nucleic acid sequences is affected by another. For example, a promoter is operably linked with a coding sequence when the promoter is capable of effecting the expression of that coding sequence (e.g., the coding sequence is under the transcriptional control of the promoter). A coding sequence may be operably linked to a regulatory sequence in a sense or antisense orientation.

The term “expression”, as used herein, may refer to the transcription and stable accumulation of sense (mRNA) or antisense RNA derived from a DNA. Expression may also refer to translation of mRNA into a polypeptide. As used herein, the term “overexpression” refers to expression that is higher than endogenous expression of the same gene or a related gene. Thus, a heterologous gene is “overexpressed” if its expression is higher than that of a comparable endogenous gene.

As used herein, the term “transformation” or “transforming” refers to the transfer and integration of a nucleic acid or fragment thereof into a host organism, resulting in genetically stable inheritance. Host organisms containing a transforming nucleic acid are referred to as “transgenic,” “recombinant,” or “transformed” organisms.

The terms “plasmid” and “vector”, as used herein, refer to an extra chromosomal element that may carry one or more gene(s) that are not part of the central metabolism of the cell. Plasmids and vectors typically are circular double-stranded DNA molecules. However, plasmids and vectors may be linear or circular nucleic acids, of a single- or double-stranded DNA or RNA, and may carry DNA derived from essentially any source, in which a number of nucleotide sequences have been joined or recombined into a unique construction that is capable of introducing a promoter fragment and a coding DNA sequence along with any appropriate 3′ untranslated sequence into a cell. In examples, plasmids and vectors may comprise autonomously replicating sequences for propagation in bacterial hosts.

“Polypeptide” and “protein” are used interchangeably herein and include a molecular chain of two or more amino acids linked through peptide bonds. The terms do not refer to a specific length of the product. Thus, “peptides”, and “oligopeptides”, are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide. The terms also include molecules in which one or more amino acid analogs or non-canonical or unnatural amino acids are included as can be synthesized, or expressed recombinantly using known protein engineering techniques. In addition, inventive fusion proteins can be derivatized as described herein by well-known organic chemistry techniques.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a recombinant host cell as a single protein.

Expression “control sequences” refers collectively to promoter sequences, ribosome binding sites, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

“Recombination” refers to the reassortment of sections of DNA or RNA sequences between two DNA or RNA molecules. “Homologous recombination” occurs between two DNA molecules which hybridize by virtue of homologous or complementary nucleotide sequences present in each DNA molecule.

The terms “stringent conditions” or “hybridization under stringent conditions” refers to conditions under which a probe will hybridize preferentially to its target subsequence, and to a lesser extent to, or not at all to, other sequences. “Stringent hybridization” and “stringent hybridization wash conditions” in the context of nucleic acid hybridization experiments such as Southern and Northern hybridizations are sequence dependent, and produce different results under varying experimental parameters. An extensive guide to the hybridization of nucleic acids is found in Tijssen (1993) Laboratory Techniques in Biochemistry and Molecular Biology—Hybridization with Nucleic Acid Probes, Part I, Chapter 2: Overview of principles of hybridization and the strategy of nucleic acid probe assays, Elsevier, New York. Generally, “highly stringent conditions” result in the hybridization of a probe to a polynucleotide sequence, wherein the probe and polynucleotide sequence share at least 85% sequence identity. The “highly stringent conditions” include stringent hybridization and wash conditions that are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. “Very highly stringent conditions” result in the hybridization of a probe to a polynucleotide sequence, wherein the probe and polynucleotide sequence share at least 95% sequence identity. The “very highly stringent conditions” include stringent hybridization and wash conditions that are selected to be equal to the Tm for a particular probe.

An example of stringent hybridization conditions for hybridization of complementary nucleic acids which have more than 100 complementary residues on a filter in a Southern or Northern blot is 50% formamide with 1 mg of heparin at 42° C., with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, Sambrook et al. (1989) Molecular Cloning—A Laboratory Manual (2nd ed.) Vol. 1-3, Cold Spring Harbor Laboratory, Cold Spring Harbor Press, NY, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. In general, a signal to noise ratio of 2× (or higher) than that observed for an unrelated probe in the particular hybridization assay indicates detection of a specific hybridization. Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

The disclosure also relates to a polynucleotide probe hybridizable under stringent conditions, and in some instances under highly stringent conditions, and in further instances under very highly stringent conditions to a polynucleotide as of the present disclosure.

As used herein, the term “hybridizing” is intended to describe conditions for hybridization and washing under “stringent conditions” for which nucleotide sequences at least about 50%, at least about 60%, at least about 70%, more preferably at least about 80% identical to each other typically remain hybridized to each other. As used herein, the term “hybridizing” is intended to describe conditions for hybridization and washing under “highly stringent conditions” for which nucleotide sequences at least about 85%, at least about 90%, identical to each other typically remain hybridized to each other. As used herein, the term “hybridizing” is intended to describe conditions for hybridization and washing under “very highly stringent conditions” for which nucleotide sequences at least about 95%, at least about 99%, identical to each other typically remain hybridized to each other.

In some embodiments an isolated nucleic acid molecule of the disclosure that hybridizes under highly stringent conditions to a nucleotide sequence of the disclosure can correspond to a naturally-occurring nucleic acid molecule. As used herein, a “naturally-occurring” nucleic acid molecule refers to an RNA or DNA molecule having a nucleotide sequence that occurs in nature (e.g., encodes a natural protein).

A skilled artisan will know which conditions to apply for stringent and highly stringent hybridization conditions. Additional guidance regarding such conditions is readily available in the art, for example, in Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.).

The terms “homology” or “percent identity” are used interchangeably herein. For the purpose of this disclosure, it is defined here that in order to determine the percent identity of two amino acid sequences or of two nucleic acid sequences, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions×100). Preferably, the two sequences are the same length.

The skilled person will be aware of the fact that several different computer programs are available to determine the homology between two sequences. For instance, a comparison of sequences and determination of percent identity between two sequences may be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available on the internet at the accelrys website, more specifically at http://www.accelrys.com), using either a Blossom 62 matrix or a PAM250 matrix, and a gap weight of 16, 14, 12, 10, 8, 6 or 4 and a length weight of 1, 2, 3, 4, 5 or 6. The skilled person will appreciate that all these different parameters will yield slightly different results but that the overall percentage identity of two sequences is not significantly altered when using different algorithms.

In yet another embodiment, the percent identity between two nucleotide sequences is determined using the GAP program in the GCG software package (available on the internet at the accelrys website, more specifically at http://www.accelrys.com), using a NWSgapdna.CMP matrix and a gap weight of 40, 50, 60, 70 or 80 and a length weight of 1, 2, 3, 4, 5 or 6. In another embodiment, the percent identity between two amino acid or nucleotide sequences is determined using the algorithm of E. Meyers and W. Miller (CABIOS, 4: 11-17 (1989) which has been incorporated into the ALIGN program (version 2.0) (available on the internet at the vega website, more specifically ALIGN-IGH Montpellier, or more specifically at http://vega.igh.cnrs.fr/bin/align-guess.cgi) using a PAM120 weight residue table, a gap length penalty of 12 and a gap penalty of 4.

The nucleic acid and protein sequences of the present disclosure may further be used as a “query sequence” to perform a search against public databases to, for example, identify other family members or related sequences. Such searches may be performed using the BLASTN and BLASTX programs (version 2.0) of Altschul, et al. (1990) J. Mol. Biol. 215:403-10. BLAST nucleotide searches may be performed with the BLASTN program, score=100, word length=12 to obtain nucleotide sequences identical to the nucleic acid molecules of the present disclosure. BLAST protein searches may be performed with the BLASTX program, score=50, word length=3 to obtain amino acid sequences identical to the protein molecules of the present disclosure. To obtain gapped alignments for comparison purposes, Gapped BLAST may be utilized as described in Altschul et al., (1997) Nucleic Acids Res. 25 (17): 3389-3402. When utilizing BLAST and Gapped BLAST programs, the default parameters of the respective programs (e.g., BLASTX and BLASTN) may be used. (Available on the internet at the ncbi website, more specifically at www.ncbi.nlm.nih.gov).

The term “motif” refers to short regions of conserved sequences of nucleic acids or amino acids that comprise part of a longer sequence.

The term “variant” refers to substantially similar sequences. Generally, nucleic acid sequence variants of the invention will have at least 46%, 48%, 50%, 52%, 53%, 55%, 60%, 65%, 70%, 75%, 76%, 77%, 78%, 79%, 80%, 85%, 86%, 87%, 88%, 89%, 90%, 91%, 92%, 93%, 94%, 95%, 96%, 97%, 98% or 99% sequence identity to the native nucleotide sequence, wherein the % sequence identity is based on the entire sequence and is determined by GAP 10 analysis using default parameters. Generally, polypeptide sequence variants of the invention will have at least about 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to the native protein, wherein the % sequence identity is based on the entire sequence and is determined by GAP 10 analysis using default parameters. GAP uses the algorithm of Needleman and Wunsch (J. Mol. Biol. 48:443-453, 1970) to find the alignment of two complete sequences that maximizes the number of matches and minimizes the number of gaps.

The term “variant” also refers to substantially similar sequences that contain amino acid sequences highly similar to the motifs contained within the invention and optionally required for the biological function of the invention. Generally, polypeptide sequence variants of the invention will have at least 85%, 90% or 95% sequence identity to the conserved amino acid residues in the defined motifs.

Variants included in the invention may contain individual substitutions, deletions or additions to the nucleic acid or polypeptide sequences which alter, add or delete a single amino acid or a small percentage of amino acids in the encoded sequence. A “conservatively modified variant” is an alteration which results in the substitution of an amino acid with a chemically similar amino acid. When the nucleic acid is prepared or altered synthetically, advantage can be taken of known codon preferences of the intended host. The nucleic acid fragments of the instant invention may be used to isolate cDNAs and genes encoding proteins with homology or sequence identity from the same or other species. Isolation of homologous genes or genes with levels of shared sequence identity using sequence-dependent protocols is well known in the art. Examples of sequence-dependent protocols include, but are not limited to, methods of nucleic acid hybridization, and methods of DNA and RNA amplification as exemplified by various uses of nucleic acid amplification technologies (e.g., polymerase chain reaction, ligase chain reaction).

For example, genes encoding other glycine N-acyltransferases, either as cDNAs or genomic DNAs, could be isolated directly by using all or a portion of the instant nucleic acid fragments as DNA hybridization probes to screen libraries from any desired organism employing methodology well known to those skilled in the art. Specific oligonucleotide probes based upon the instant nucleic acid sequences can be designed and synthesized by methods known in the art (Sambrook). Moreover, the entire sequences can be used directly to synthesize DNA probes by methods known to the skilled artisan such as random primer DNA labeling, nick translation, or end-labeling techniques, or RNA probes using available in vitro transcription systems. In addition, specific primers can be designed and used to amplify a part or all of the instant sequences. The resulting amplification products can be labeled directly during amplification reactions or labeled after amplification reactions, and used as probes to isolate full length cDNA or genomic fragments under conditions of appropriate stringency.

Strategies for designing and constructing variant genes and proteins that comprise contiguous residues of a particular molecule can be determined by obtaining and examining the structure of a protein of interest (e.g., atomic 3-D (three dimensional) coordinates from a crystal structure and/or a molecular model). In some examples, a strategy may be directed to certain segments of a protein that are ideal for modification, such as surface-exposed segments, and not internal segments that are involved with protein folding and essential 3-D structural integrity. U.S. Pat. No. 5,605,793, for example, relates to methods for generating additional molecular diversity by using DNA reassembly after random or focused fragmentation. This can be referred to as gene “shuffling”, which typically involves mixing fragments (of a desired size) of two or more different DNA molecules, followed by repeated rounds of renaturation. This process may improve the activity of a protein encoded by a subject gene. The result may be a chimeric protein having improved activity, altered substrate specificity, increased enzyme stability, altered stereospecificity, or other characteristics.

An amino acid “substitution” can be the result of replacing one amino acid in a reference sequence with another amino acid having similar structural and/or chemical properties (i.e., conservative amino acid substitution), or it can be the result of replacing one amino acid in a reference sequence with an amino acid having different structural and/or chemical properties (i.e., non-conservative amino acid substitution). Amino acids can be placed in the following structural and/or chemical classes: non-polar, uncharged polar; basic; and acidic. Accordingly, “conservative” amino acid substitutions can be made on the basis of similarity in polarity, charge, solubility, hydrophobicity, hydrophilicity, or the amphipathic nature of the residues involved. For example, non-polar (hydrophobic) amino acids include glycine, alanine, leucine, isoleucine, valine, proline, phenylalanine, tryptophan, and methionine; uncharged (neutral) polar amino acids include serine, threonine, cysteine, tyrosine, asparagine, and glutamine; positively charged (basic) amino acids include arginine, lysine, and histidine; and negatively charged (acidic) amino acids include aspartic acid and glutamic acid. Alternatively, “non-conservative” amino acid substitutions can be made by selecting the differences in the polarity, charge, solubility, hydrophobicity, hydrophilicity, or amphipathic nature of any of these amino acids. “Insertions” or “deletions” can be within the range of variation as structurally or functionally tolerated by the recombinant proteins.

In some embodiments, a variant protein is “truncated” with respect to a reference, full-length protein. In some examples, a truncated protein retains the functional activity of the reference protein. By “truncated” protein, it is meant that a portion of a protein may be cleaved off, for example, while the remaining truncated protein retains and exhibits the desired activity after cleavage. Cleavage may be achieved by any of various proteases. Furthermore, effectively cleaved proteins can be produced using molecular biology techniques, wherein the DNA bases encoding a portion of the protein are removed from the coding sequence, either through digestion with restriction endonucleases or other techniques available to the skilled artisan. A truncated protein may be expressed in a heterologous system, for example, B. subtilis, E. coli, baculoviruses, plant-based viral systems, and yeast. Truncated proteins conferring glycine N-acyltransferase activity may be confirmed by using the heterologous expression system expressing the proteins, such as described herein. It is well-known in the art that truncated proteins can be successfully produced so that they retain the functional activity of the full-length reference protein. For example, Bt proteins can be used in a truncated (core protein) form. See, e.g., Hofte and Whiteley (1989) Microbiol. Rev. 53(2):242-55; and Adang et al. (1985) Gene 36:289-300.

In some cases, especially for expression in bacterial strains, it can be advantageous to use truncated genes that express truncated proteins. Truncated genes may encode a polypeptide comprised of, for example, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% of the full-length protein. The variant genes and proteins that retain the function of the reference sequence from which they were designed may be determined by one of skill in the art, for example, by assaying recombinant variants for activity. If such an activity assay is known and characterized, then the determination of functional variants requires only routine experimentation.

Specific changes to the “active site” of an enzyme may be made to affect the inherent functionality with respect to activity or stereospecificity. See, Muller et. al. (2006) Protein Sci. 15(6): 1356-68. For example, the known tauD structure has been used as a model dioxygenase to determine active site residues while bound to its inherent substrate, taurine. See, Elkins et al. (2002) Biochemistry 41(16):5185-92. Further information regarding sequence optimization and designability of enzyme active sites can be found in Chakrabarti et al. (2005) Proc. Natl. Acad. Sci. USA 102(34):12035-40.

Various structural properties and three-dimensional features of a protein may be changed without adversely affecting the activity/functionality of the protein. Conservative amino acid substitutions can be made that do not adversely affect the activity and/or three-dimensional configuration of the molecule (“tolerated” substitutions). Variant proteins can also be designed that differ at the sequence level from the reference protein, but which retain the same or similar overall essential three-dimensional structure, surface charge distribution, and the like. See, e.g., U.S. Pat. No. 7,058,515; Larson et al. (2002) Protein Sci. 1 1:2804-13; Crameri et al. (1997) Nat. Biotechnol. 15:436-8; Stemmer (1994) Proc. Natl. Acad. Sci. USA 91:10747-51; Stemmer (1994) Nature 370:389-91; Stemmer (1995) Bio/Technology 13:549-53; Crameri et al. (1996) Nat. Med. 2:100-3; and Crameri et al. (1996) Nat. Biotechnol. 14: 315-9.

The term “chimeric” as used herein, means comprised of sequences that are “recombined”. For example the sequences are recombined and are not found together in nature.

The term “recombine” or “recombination” as used herein means refers to any method of joining polynucleotides. The term includes end to end joining, and insertion of one sequence into another. The term is intended to encompass includes physical joining techniques such as sticky-end ligation and blunt-end ligation. Such sequences may also be artificially, or recombinantly synthesized to contain the recombined sequences. Additionally, the term can encompass the integration of one sequence within a second sequence, for example the integration of a polynucleotide within the genome of an organism by homologous recombination can result from “recombination”.

### III. Embodiments of the Present Disclosure

In an embodiment, the subject disclosure relates to prokaryotic microorganisms that are metabolically engineered to produce non-native lipoamino acid, N-acylglycine biosurfactants. Prokaryotic microorganisms can be utilized for production of novel compounds via fermentation in cultures. As such, the microorganism is metabolically-engineered via recombinant DNA technology for the production of the desired chemical compound. The subject disclosure describes a process to utilize recombinant DNA technology to design and express glycine N-acyltransferase proteins for the production of an N-acylglycine biosurfactant within a prokaryotic microorganism.

In certain embodiments the biosurfactant is a metabolic product produced by a microorganism. The biosurfactant molecules are composed of two distinct moieties: a hydrophilic and a hydrophobic moiety. Biosurfactants can be categorized as glycolipids (a carbohydrate linked to a fatty acid), proteolipids (an amino acid or chain of amino acids linked to a fatty acid), or polymeric surfactants (high molecular weight structures consisting of fatty acids). The metabolic product may be a fatty acid, and in some instances the surfactant is a beta-hydroxy fatty acid. Typically, the biosurfactant is biodegradable, less toxic, and produced more efficiently than synthetic compounds produced from chemical refinement of a feedstock (i.e., petrochemical feed stocks).

Various strains of microorganisms are capable of producing surfactants. For example; Bacillus sp. (i.e., Bacillus subtilis), Mycobacterium sp., Corynebacterium sp., Ustilago sp., Arthrobacter sp., Candida sp., Pseudomonas sp., Torulopsis sp., Escherchia sp. and Rhodococcus sp. are only a few of the many various types of microorganisms that can naturally produce surfactants. In an embodiment, the metabolically engineered microorganism of the subject disclosure can comprise a Bacillus sp., Mycobacterium sp., Corynebacterium sp., Ustilago sp., Arthrobacter sp., Candida sp., Pseudomonas sp., Torulopsis sp., Escherchia sp., and Rhodococcus sp. In further embodiments, the metabolically engineered microorganism of the subject disclosure can comprise a yeast microorganism, a cyanobacterium microorganism, or a bacterial microorganism. Generally, bacterial microorganisms are categorized by differentiating bacterial species into gram positive or gram negative species. The gram staining is used to identify bacterial strains that contain peptidoglycan in the cell wall. This microbiological procedure is commonly known in the art, and would be appreciated as a common categorical process by those persons having ordinary skill in the art.

Heterologous expression of an enzyme and production of a biosurfactant in certain species of microorganisms can result in altered properties of the biosurfactant. For example, the chain length of the fatty acid of a biosurfactant may vary, in part, due to the microorganism that it is produced from. In addition, the fatty acid chain may be branched or contain additional chemical moieties (i.e., hydroxylation, acylation, alkylation, oxidation, etc.) thereby altering the chemical structure of the fatty acid moiety of a biosurfactant and further altering the functionality of the biosurfactant (i.e., length of fatty acid, charge, solubility in water, molecular weight, etc.). The microorganism from which the biosurfactant is produced will impart such properties on the biosurfactant. In certain embodiments of the subject disclosure, the microorganism is engineered to acylate an amino acid (i.e., glycine) to a biosurfactant. In such embodiments, the microorganism is metabolically engineered to acylate the amino acid (i.e., glycine) to the biosurfactant.

In certain embodiments the amino acid, glycine, is acylated to a fatty acid (i.e., acyl-coA) to produce an N-acylglycine biosurfactant. In certain embodiments, the amino acid glycine is recruited into a medium chain-length β-hydroxy fatty acid peptide chain. “Beta-hydroxy fatty acids” are fatty acids (i.e., acyl-coA) comprising a hydroxy group at the third carbon (i.e., the beta position) of the fatty acid chain. Typically, the carboxylate moiety of the fatty acid is covalently attached to the nitrogen of the amino acid such that the beta position corresponds to the carbon two carbons removed from the carbon having the ester group. “Medium chain length” beta-hydroxy fatty acids may be in length of 3, 4, 5, 6, 7, 8, 9, 10, 11, 12, 3, 14, 15, 16, 17, 18, 19, 20, 21, 22, 23, 24, 25 or more carbon atoms. In some embodiments the amino acid glycine is linked to the beta-hydroxy fatty acids to produce an N-acylglycine surfactant in the length of 3, 4, 5, 6, 7, 8, 9, 10, 11, 12, 3, 14, 15, 16, 17, 18, 19, 20, 21, 22, 23, 24, 25 or more carbon atoms. In additional embodiments the amino acid glycine is covalently linked to the beta-hydroxy fatty acids to produce an N-acylglycine surfactant in the length of 3, 4, 5, 6, 7, 8, 9, 10, 11, 12, 3, 14, 15, 16, 17, 18, 19, 20, 21, 22, 23, 24, 25 or more carbon atoms.

In additional embodiments, the N-acylglycine surfactant may contain linear carbon chains, in which each carbon of the chain, with the exception of the terminal carbon atom and the carbon attached to the nitrogen of the amino acid, is directly covalently linked to two other carbon atoms. Additionally or alternatively, N-acylglycine surfactant may contain branched carbon chains, in which a least one carbon of the chain is directly covalently linked to three or more other carbon atoms. N-acylglycine surfactant may contain one or more double bonds between adjacent carbon atoms. Alternatively, N-acylglycine surfactant may contain only single-bonds between adjacent carbon atoms. Furthermore, different beta-hydroxy fatty acid linkage domains that exhibit specificity for other beta-hydroxy fatty acids (e.g., naturally or non-naturally occurring beta-hydroxy fatty acids) may be used to generate the N-acylglycine surfactant.

The fatty acid of a microorganism can vary, depending upon the bacterial strain, growth media, cultivation conditions, etc. Most bacteria produce straight-chain fatty acids with or without unsaturation in the carbon chain (myristic, palmitic, stearic, oleic, and linoleic acids). Branched-chain fatty acids with a methyl group at the penultimate (iso-) or the antepenultimate (anteiso-) positions are relatively uncommon but are the major constituents of lipids in gram positive bacteria such as Bacillus subtilis.

In B. subtilis, branched-chain fatty acids account for >90% of the total fatty acid pool (Roberts, 1994, USB, B. mojavensis—distinguishable from B. subtilis, V44(2), p. 256-264). Anteiso-fatty acids (anteiso-C15 and anteiso-C17 at about 40.19%±3.98% and 9.38%±0.95%, respectively) are the most abundant, with anteiso-C15 fatty acids being the single most abundant fatty acid in B. subtilis. The odd-numbered, iso-fatty acids (iso-C15 and iso-C17 at about 29.27%±4.64% and 9.59%±1.56%, respectively) are next in order of abundance. The even-numbered, iso-(iso-C14 and iso-C16 at about 1.13%±0.24% and 2.36%±0.34%, respectively) and straight-chain (n-C14 at about a concentration not currently measured and n-C16 at about 3.14%±0.40%) fatty acids are of relatively low abundance. Unsaturated fatty acids account for a small fraction of the lipid content in B. subtilis with C16:1 cis9, C16:1 cis5, and iso-C17:1 cis7 at about 0.23%±0.35%, 1.52%±0.45%, and 1.72%±0.42%, respectively.

The observed trend in fatty composition in B. subtilis is also generally conserved across other species within the Bacillus genus such as B. alvei, B. amyloliquefaciens, B. atrophaeus, B. brevis, B. circulans, B. licheniformis, B. macerans, B. megaterium and B. pumilus (Kaneda, 1967, J. Bac., Fatty acids in the Genus Bacillus: Iso- and anteiso-fatty acids as characteristic constituents, V93(3), p. 894-903). Anteiso-fatty acids (anteiso-C15 and anteiso-C17) are typically the most abundant and anteiso-C15 fatty acid is the single most abundant fatty acid in B. subtilis. The odd-numbered iso-fatty acids (iso-C15 and iso-C17) are next in order of abundance, and the even-numbered iso-(iso-C14 and iso-C16) and straight-chain (n-C14 and n-C16) fatty acids are of relatively low and variable abundance, respectively.

In E. coli, the majority of the fatty acids produced are straight-chain and range from C14-C18 in carbon length (Sullivan, 1979, J. Bac., Alteration of FA composition of E. coli by growth in presence of alcohols, V138(1), p. 133-138; Shaw, 1965, J Bac, Fatty acid composition of E. coli as a possible control factor of minimal growth temperature, V90(1), p. 141-146). The fatty acids of C16 length (C16:0 at about 30.95-38.6% and C16:1 at about 27.9-31.45%) are the most abundant pair of acids in E. coli. Unsaturated, C18 fatty acid (C18:1 at about 19.5-27.1%) is next in order of abundance while C14 and C17 fatty acids were of relatively low abundance at about 5.1-5.5% and 3-4.9%, respectively.

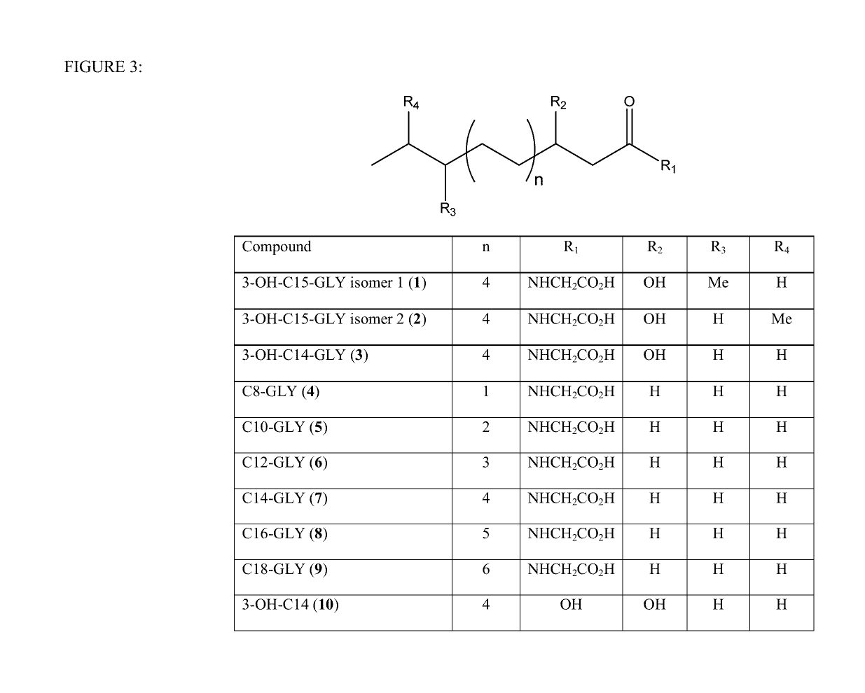

In embodiments the N-acylglycine surfactant produced in a microorganism can be composed of an N-acylglycine surfactant comprising, but not limited to: anteiso-C15-N-acylglycine surfactant; anteiso-C17-N-acylglycine surfactant; iso-C15-N-acylglycine surfactant; iso-C17-N-acylglycine surfactant; iso-C14-N-acylglycine surfactant; iso-C16-N-acylglycine surfactant; straight-chain-C14-N-acylglycine surfactant; straight-chain-C16-N-acylglycine surfactant; straight-chain-C17-N-acylglycine surfactant; C16:1 cis9-N-acylglycine surfactant; C16:1 cis5-N-acylglycine surfactant; unsaturated-C18:1-N-acylglycine surfactant; C16-N-acylglycine surfactant; C16:1-N-acylglycine surfactant; and, iso-C17:1 cis7-N-acylglycine surfactant. In further embodiments, the N-acylglycine surfactant produced in a microorganism can be composed of an N-acylglycine surfactant comprising the 3-OH—C15-GLY isomer 1-N-acylglycine surfactant of FIG. 3; the 3-OH—C15-GLY isomer 2-N-acylglycine surfactant of FIG. 3; C8-GLY 4-N-acylglycine surfactant of FIG. 3; C10-GLY 5-N-acylglycine surfactant of FIG. 3; C12-GLY 6-N-acylglycine surfactant of FIG. 3; C14-GLY 7-N-acylglycine surfactant of FIG. 3; C16-GLY 8-N-acylglycine surfactant of FIG. 3; C18-GLY 9-N-acylglycine surfactant of FIG. 3; and 3-OH—C14 10-N-acylglycine surfactant of FIG. 3. As described above, the pool of fatty acids are known to be produced in the microorganism, and can serve as a pool of fatty acids that can be converted by a glycine N-Acyltransferase enzymatic protein of the subject disclosure into an N-acylglycine surfactant, wherein the N-acylglycine surfactant is comprised of a varying chain lengths, branching and addition of chemical moieties. The production of such N-acylglycine surfactant molecules are taught herein as an embodiment of the subject disclosure.

A glycine N-Acyltransferase protein can be an enzyme that can selectively bind and condense the amino acid glycine into a medium chain-length β-hydroxy fatty acid peptide chain. Unexpectedly, the heterologous expression of the Glycine N-Acyltransferase protein in a microorganism successfully enabled the in vivo acylation of the amino acid glycine into a medium chain-length β-hydroxy fatty acid peptide chain. As such, when the Glycine N-Acyltransferase protein was expressed in a prokaryotic species, the bacterial strain was cultured and fermented to result in the production of the non-native lipoamino acid, N-acylglycine biosurfactant. Accordingly, embodiments of the subject disclosure are Glycine N-Acyltransferase proteins and polynucleotides which encode such proteins.

In an embodiment, the subject disclosure provides a protein sequence that catalyzes conjugation of glycine with a β-hydroxy fatty acid to produce N-acylglycine biosurfactants. Representative Glycine N-Acyltransferase proteins that catalyze the reaction are disclosed herein. An exemplary Glycine N-Acyltransferase is the Glycine N-Acyltransferase protein of SEQ ID NO:1. Further, embodiments include protein sequences that share at least 85%, 87.5%, 90%, 92.5%, 95%, 97.5%, 99%, or 99.5% sequence similarity to SEQ ID NO:1. Another exemplary Glycine N-Acyltransferase is the Glycine N-Acyltransferase-Like 1 protein of SEQ ID NO:3. Further, embodiments include protein sequences that share at least 85%, 87.5%, 90%, 92.5%, 95%, 97.5%, 99%, or 99.5% sequence similarity to SEQ ID NO:3. Yet another exemplary Glycine N-Acyltransferase is the Glycine N-Acyltransferase-Like 2 protein of SEQ ID NO:5. Further, embodiments include protein sequences that share at least 85%, 87.5%, 90%, 92.5%, 95%, 97.5%, 99%, or 99.5% sequence similarity to SEQ ID NO:5. Further exemplary Glycine N-Acyltransferase is the Glycine N-Acyltransferase-Like 3 protein of SEQ ID NO:7. Further, embodiments include protein sequences that share it least 85%, 87.5%, 90%, 92.5%, 95%, 97.5%, 99%, or 99.5% sequence similarity to SEQ ID NO:7.

Further provided within this disclosure are polynucleotides encoding the Glycine N-Acyltransferase. Exemplary polynucleotides include native polynucleotides that are operably linked with a promoter regulatory region for the expression of the polynucleotide within a microorganism. In an embodiment, the polynucleotide may share at least 85%, 87.5%, 90%, 92.5%, 95%, 97.5%, 99%, or 99.5% sequence similarity to the polynucleotide of SEQ ID NO:2. In further embodiments, the polynucleotide may share at least 85%, 87.5%, 90%, 92.5%, 95%, 97.5%, 99%, or 99.5% sequence similarity to the polynucleotide of SEQ ID NO:4. In additional embodiments, the polynucleotide may share at least 85%, 87.5%, 90%, 92.5%, 95%, 97.5%, 99%, or 99.5% sequence similarity to the polynucleotide of SEQ ID NO:6. In embodiments, the polynucleotide may share at least 85%, 87.5%, 90%, 92.5%, 95%, 97.5%, 99%, or 99.5% sequence similarity to the polynucleotide of SEQ ID NO:8.

A native polynucleotide may be heterologously expressed in a non-native organism. Such heterologous expression of the native polynucleotide may be optimized by re-building the native polynucleotide to include a codon distribution that is more representative of the non-native organism in which the polynucleotide shall be expressed. Disclosed herein are codon optimized sequences for the expression of a polynucleotide encoding a Glycine N-Acyltransferase protein within a microorganism. In an embodiment, the Glycine N-Acyltransferase encoding polynucleotide may be codon optimized to share the codon usage of a bacterial species. In an embodiment, the Glycine N-Acyltransferase encoding polynucleotide may be codon optimized to share the codon usage of a Escherichia sp. microorganism. In an embodiment, the Glycine N-Acyltransferase encoding polynucleotide may be codon optimized to share the codon usage of a Bacillus sp. microorganism. As such, an embodiment of the subject disclosure includes Glycine N-Acyltransferase codon optimized polynucleotide sequences that shares at least 85%, 87.5%, 90%, 92.5%, 95%, 97.5%, 99% or 99.5 with SEQ ID NO:9. A further embodiment of the subject disclosure includes Glycine N-Acyltransferase-like 1 codon optimized polynucleotide sequences that shares at least 85%, 87.5%, 90%, 92.5%, 95%, 97.5%, 99% or 99.5 with SEQ ID NO:10. Yet another embodiment of the subject disclosure includes Glycine N-Acyltransferase-like 2 codon optimized polynucleotide sequences that shares at least 85%, 87.5%, 90%, 92.5%, 95%, 97.5%, 99% or 99.5 with SEQ ID NO: 11. An additional embodiment of the subject disclosure includes Glycine N-Acyltransferase-like 3 codon optimized polynucleotide sequences that shares at least 85%, 87.5%, 90%, 92.5%, 95%, 97.5%, 99% or 99.5 with SEQ ID NO:12.

In an embodiment, the subject disclosure relates to a protein comprising a Glycine N-Acyltransferase domain active site. An exemplary Glycine N-Acyltransferase specific domain active site is disclosed herein and includes a protein motif of P(A/E)S(L/I)KVYG(T/A/S)(V/I)(F/M/Y)(H/N)I(N/K)(H/R/D)(G/K)NPF (SEQ ID NO:9). In another embodiment, the Glycine N-Acyltransferase can be a protein motif of D(D/N)(L/Q/M)D(H/S)YTN(T/A/V)Y (SEQ ID NO:10). In a further embodiment, the Glycine N-Acyltransferase can be a protein motif of W(K/D/E)Q(H/V/T/R)(L/F)QIQ (SEQ ID NO: 11). Furthermore, in an embodiment the Glycine N-Acyltransferase can be a protein motif of L(V/L)N(K/R/E/D)(FT/H/N)W(H/S/A/K)(F/R)G(G/K)NE (SEQ ID NO:12). In yet another embodiment, the Glycine N-Acyltransferase can be a protein motif of (G/D)(P/E)(E/K)G(T/N/Q/V)(P/L)V(C/S)W (SEQ ID NO:13). These five consensus sequences were determined to define motifs that are characteristic of a Glycine N-Acyltransferase protein. Using these motifs to search databases (e.g. GeneBank), one practiced in the art may identify additional putative Glycine N-Acyltransferase genes or proteins from a variety of different organisms.

The subject disclosure relates to a variant protein comprising the activity of a Glycine N-Acyltransferase enzyme. In some embodiments, the variant having Glycine N-Acyltransferase activity possesses at least 85%, 87.5%, 90%, 92.5%, 95%, 97.5%, 99%, 99.5%, or 99.9% sequence identity with a sequence selected from SEQ ID NO: 1. In further embodiments, the variant having Glycine N-Acyltransferase activity possesses at least 85%, 87.5%, 90%, 92.5%, 95%, 97.5%, 99%, 99.5%, or 99.9% sequence identity with a sequence selected from SEQ ID NO:3. In other embodiments, the variant having Glycine N-Acyltransferase activity possesses at least 85%, 87.5%, 90%, 92.5%, 95%, 97.5%, 99%, 99.5%, or 99.9% sequence identity with a sequence selected from SEQ ID NO:5. In additional embodiments, the variant having Glycine N-Acyltransferase activity possesses at least 85%, 87.5%, 90%, 92.5%, 95%, 97.5%, 99%, 99.5%, or 99.9% sequence identity with a sequence selected from SEQ ID NO:7.

Furthermore, the subject disclosure relates to a polypeptide having Glycine N-Acyltransferase activity wherein said polypeptide is encoded by an isolated polynucleotide that hybridizes under stringent conditions with the sense or anti-sense strand of a polynucleotide probe sequence selected from SEQ ID NO:2, SEQ ID NO:4, SEQ ID NO:6, SEQ ID NO:8, SEQ ID NO: 14, or SEQ ID NO: 15. In some embodiments, the polypeptide having Glycine N-Acyltransferase activity wherein said polypeptide is encoded by an isolated polynucleotide that hybridizes under highly stringent conditions with the sense or anti-sense strand of a polynucleotide probe sequence selected from SEQ ID NO:2, SEQ ID NO:4, SEQ ID NO:6, SEQ ID NO:8, SEQ ID NO:14, or SEQ ID NO:15.

In a further embodiment of the subject disclosure the Glycine N-Acyltransferase protein is encoded on a polynucleotide construct of SEQ ID NO:19, SEQ ID NO:20, SEQ ID NO:21, SEQ ID NO:22, SEQ ID NO:31, or SEQ ID NO:32. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO: 19. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO:20. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO:21. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO:22. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO:31. In a subsequent embodiment, a Glycine N-Acyltransferase polynucleotide shares at least 85%, 87.5%, 90%, 92.5%, 95%, 97.5%, 99% or 99.5% with the Glycine N-Acyltransferase coding sequence of SEQ ID NO:32. In other embodiments, the Glycine N-Acyltransferase coding sequence of SEQ ID NO:2, SEQ ID NO:4, SEQ ID NO:6, SEQ ID NO:8, SEQ ID NO: 14, or SEQ ID NO: 15 are operatively linked to a ribosome binding sequence. In subsequent embodiments, the ribosome binding sequence of SEQ ID NO: 17 can be operably linked to SEQ ID NO:2, SEQ ID NO:4, SEQ ID NO:6, SEQ ID NO:8, SEQ ID NO: 14, or SEQ ID NO: 15. In further embodiments, the Glycine N-Acyltransferase coding sequence of SEQ ID NO:2, SEQ ID NO:4, SEQ ID NO:6, SEQ ID NO:8, SEQ ID NO:14, or SEQ ID NO:15 are operatively linked to a terminator sequence. In subsequent embodiments, the terminator sequence of SEQ ID NO:18 can be operably linked to SEQ ID NO:2, SEQ ID NO:4, SEQ ID NO:6, SEQ ID NO:8, SEQ ID NO:14, or SEQ ID NO:15. In embodiments, the Glycine N-Acyltransferase coding sequence of SEQ ID NO:2, SEQ ID NO:4, SEQ ID NO:6, SEQ ID NO:8, SEQ ID NO: 14, or SEQ ID NO: 15 are operatively linked to a bacterial promoter sequence. In subsequent embodiments, the bacterial promoter sequence of SEQ ID NO:16 can be operably linked to SEQ ID NO:2, SEQ ID NO:4, SEQ ID NO:6, SEQ ID NO:8, SEQ ID NO:14, or SEQ ID NO:15.

In an embodiment of the subject disclosure, expression of the glycine N-acyltransferase gene is driven by a bacterial promoter. Exemplary promoters are known to those with ordinary skill in the art, and may include a pTAC promoter, a LAC promoter, TAC II promoter, or a PsrfA promoter amongst other commonly known bacterial promoters. Exemplary promoters may be constitutive or inducible. In an embodiment the glycine N-acyltransferase gene is operably linked to a bacterial promoter. In some embodiment the bacterial promoter is a Pspac promoter of SEQ ID NO:16.

In further embodiments, a glycine N-acyltransferase gene operably linked to a bacterial promoter may be cloned into a vector that can then be transformed into the bacterial host cell. Other regulatory elements may be included in a vector (also termed “expression construct”). Such elements include, but are not limited to, for example, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” polypeptide coding sequences, which facilitates identification, separation, purification, and/or isolation of an expressed.

A polypeptide encoding gene according to the present disclosure can include, in addition to the protein coding sequence, the following regulatory elements operably linked thereto: a promoter, a ribosome binding site (RBS), a transcription terminator, translational start and stop signals. Useful RBSs can be obtained from any of the species useful as host cells in expression systems according to the present disclosure, preferably from the selected host cell. Many specific and a variety of consensus RBSs are known, e.g.; those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12): 1123-30 (December 2001). In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989)(native RBS sequence of AAGGAAG). Further examples of methods, vectors, and translation and transcription elements, and other elements useful in the present disclosure are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to protein Wilcox. In a further embodiment, the RBS can be a sequence of SEQ ID NO:17.

Vectors are known in the art for expressing recombinant proteins in host cells, and any of these may be used for expressing the genes according to the present disclosure. The plasmid vectors may autonomously replicate within the bacterial strain with or without the use of an antibiotic selection agent. Such vectors include, e.g., plasmids, cosmids, and phage expression vectors. Examples of useful plasmid vectors include, but are not limited to, the expression plasmids pBBR1MCS, pDSK519, pKT240, pML122, pPS10, RK2, RK6, pRO1600, and RSF1010. Further examples can include pALTER-Ex1, pALTER-Ex2, pBAD/His, pBAD/Myc-His, pBAD/gIII, pCal-n, pCal-n-EK, pCal-c, pCal-Kc, pcDNA 2.1, pDUAL, pET-3a-c, pET 9a-d, pET-11a-d, pET-12a-c, pET-14b, pET15b, pET-16b, pET-17b, pET-19b, pET-20b(+), pET-21a-d(+), pET-22b(+), pET-23a-d(+), pET24a-d(+), pET-25b(+), pET-26b(+), pET-27b(+), pET28a-c(+), pET-29a-c(+), pET-30a-c(+), pET31b(+), pET-32a-c(+), pET-33b(+), pET-34b(+), pET35b(+), pET-36b(+), pET-37b(+), pET-38b(+), pET-39b(+), pET-40b(+), pET411a-c(+), pET-42a-c(+pET43a-c(+), pETBlue-1, pETBlue-2, pETBlue-3, pGEMEX-1, pGEMEX-2, pGEX1λT, pGEX-2T, pGEX-2TK, pGEX-3X, pGEX4T, pGEX-5X, pGEX-6P, pHAT10/11/12, pHAT20, pHAT-GFPuv, pKK223-3, pLEX, pMAL-c2X, pMAL-c2E, pMAL-c2g, pMAL-p2X, pMAL-p2E, pMAL-p2G, pProEX HT, pPROLar.A, pPROTet.E, pQE-9, pQE-16, pQE-30/31/32, pQE40, pQE-50, pQE-70, pQE-80/81/82L, pQE-100, pRSET, and pSE280, pSE380, pSE420, pThioHis, pTrc99A, pTrcHis, pTrcHis2, pTriEx-1, pTriEx-2, pTrxFus. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner & W. Wackemagel, in Biomolec. Eng. 17(1):11-16. (October 2000); H. P. Schweizer, in Curr. Opin. Biotech. 12(5):439-45 (October 2001); M. Bagdasarian & K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich & Y. K. Fomichev, in Gene 140(1):63-65 (Mar. 11, 1994); M. Tsuda & T. Nakazawa, in Gene 136(1-2):257-62 (Dec. 22, 1993); C. Nieto et al., in Gene 87(1):145-49 (Mar. 1, 1990); J. D. Jones & N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001). In addition, Bacillus plasmids, e.g., pDG1662 plasmid, may be obtained from the Bacillus Genetic Stock Center, Biological Sciences 556, 484 W. 12th Ave, Columbus, Ohio 43210-1214.

Transformation of the host cells with the vector(s) disclosed herein may be performed using any transformation methodology known in the art, and the bacterial host cells may be transformed as intact cells or as protoplasts (i.e. including cytoplasts). Exemplary transformation methodologies include ‘poration methodologies, e.g., electroporation, protoplast fusion, bacterial conjugation, and divalent cation treatment (calcium chloride CaCl₂ treatment or CaCl₂/Mg²⁺ treatment), or other well known methods in the art. See, e.g., Morrison, J. Bact., 132:349-351 (1977); Clark-Curtiss & Curtiss, Methods in Enzymology, 101:347-362 (Wu et al., eds, 1983), Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994)). Other known transformation methods specific are described at by Guerout-Fleury, A. M., Frandsen, N. and Stragier, P. (1996) Plasmids for ectopic integration in Bacillus subtilis. Gene 180 (1-2), 57-61.

Embodiments of the disclosure include methods for identifying any neutral site within a bacterial microorganism (i.e., Bacillus subtilis) genome and the integration of a polynucleotide containing a gene expression cassette which is stably expressed.

Other embodiments of the present disclosure can include integrating a polynucleotide into a bacterial microorganism (i.e., Bacillus subtilis) genome without negatively impacting the production, growth or other desired metabolic characteristics of the bacterial microorganism (i.e., Bacillus subtilis).

Other embodiments of the present disclosure can include integrating a polynucleotide into the bacterial microorganism (i.e., Bacillus subtilis) genome at a neutral site, and the subsequent stacking of a second polynucleotide at the same location. Wherein, the neutral site within the bacterial microorganism (i.e., Bacillus subtilis) is utilized as a preferred locus for introducing additional polynucleotides. In an embodiment the amyE genomic locus serves as a neutral integration site for the integration of a polynucleotide into the bacterial microorganism (i.e., Bacillus subtilis) genome.

Other embodiments of the present disclosure can include integrating a polynucleotide containing a gene expression cassette into the bacterial microorganism (i.e., Bacillus subtilis) genome at a neutral site, and the subsequent removal of a selectable marker expression cassette from the integrated polynucleotide. Wherein, the method used to remove the selectable marker expression cassette is a double crossing over method, an excision method using CRE-LOX, an excision method using FLP-FRT, or an excision method using the RED/ET RECOMBINATION® kit (Genebridges, Heidelberg, Germany), in addition to other excision methods known in the art.

Other embodiments of the present disclosure can include integrating a polynucleotide into bacterial microorganism (i.e., Bacillus subtilis) genome at a neutral site as an alternative to the use of extraneous replicating plasmids. Wherein, one or more extraneous replicating plasmids are incompatible due to the presence of similar origins or replication, incompatibility groups, redundant selectable marker, or other gene elements. Wherein, one or more extraneous replicating plasmids are not functional in bacterial microorganism (i.e., Bacillus subtilis) due to the specificity of the bacterial microorganism (i.e., Bacillus subtilis) restriction modification system. Wherein, one or more extraneous replicating plasmids are not available, functional or readily transformable within bacterial microorganism (i.e., Bacillus subtilis).

Other embodiments of the present disclosure can include methods for increasing the efficiency of homologous recombination in a prokaryotic cell. Methods relying upon homologous recombination mediated by introduced enzymes, such as lambda red ‘recombineering’ and analogous approaches are useful in a limited number of bacterial classes, particularly Escherichia (Datsenko and Wanner (2000) Proc Natl Acad Sci USA. 97: 6640-5), Salmonella, and Bacillus. Methods relying upon site-specific recombination mediated by introduced enzymes, such as phage integrases, FLP/FRT or Cre/loxP may also be used, but are reliant on the presence of pre-existing sites within the target DNA (Wirth et al (2007) Current Opinions in Biotechnology 18, 411-419). Alternative methods exploit viruses or mobile elements, or their components (e.g. phage, transposons or mobile introns).

However, methods relying upon host-mediated homologous recombination are by far the most commonly-used type of chromosomal DNA modifications. In a typical microbial application of host-mediated homologous recombination, a plasmid with a single region of sequence identity with the chromosome is integrated into the chromosome by single-crossover integration, sometimes referred to as ‘Campbell-like integration’. After such an event, genes on the introduced plasmid are replicated as part of the chromosome, which may be more rapid than the plasmid replication. Accordingly, growth in medium with selection for a plasmid-borne selectable marker gene may provide a selective pressure for integration. Campbell-like integration can be used to inactivate a chromosomal gene by placing an internal fragment of a gene of interest on the plasmid, so that after integration, the chromosome will not contain a full-length copy of the gene. The chromosome of a Campbell-like integrant cell is not stable, because the integrated plasmid is flanked by the homologous sequences that directed the integration. A further homologous recombination event between these sequences leads to excision of the plasmid, and reversion of the chromosome to wild-type. For this reason, it may be necessary to maintain selection for the plasmid-borne selectable marker gene to maintain the integrant clone.

An improvement on the basic single-crossover integration method of chromosomal modification is double crossover homologous recombination, also referred to as allelic exchange, which involves two recombination events. The desired modified allele is placed on a plasmid flanked by regions of homology to the regions flanking the target allele in the chromosome (‘homology arms’). A first integration event can occur in either pair of homology arms, leading to integration of the plasmid into the chromosome in the same manner as Campbell-like integration. After the first crossover event, the chromosome contains two alternative sets of homologous sequences that can direct a second recombination event. If the same sequences that directed the first event recombine, the plasmid will be excised, and the cell will revert to wild-type. If the second recombination event is directed by the other homology arm, a plasmid will be excised, but the original chromosomal allele will have been exchanged for the modified allele introduced on the plasmid; the desired chromosomal modification will have been achieved. As with Campbell-like integration, the first recombination event is typically detected and integrants isolated using selective advantage conferred by integration of a plasmid-borne selectable marker gene.

As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, a mineral salts media; a rich medium may be used, but is preferably avoided. In another embodiment either a minimal medium or a mineral salts medium is selected. In still another embodiment, a minimal medium is selected. In yet another embodiment, a mineral salts medium is selected. Mineral salts media are particularly preferred. All such media can be utilized for the expression of N-acylglycine surfactants and are considered as a suitable expression medium for microorganism fermentation.

The fermentation system according to the present disclosure can be cultured in any fermentation format. For example, batch, fed-batch, semi-continuous, and continuous fermentation modes may be employed herein.

The fermentation systems according to the present disclosure are useful for transgene expression at any scale (i.e. volume) of fermentation. Thus, e.g., microliter-scale, centiliter scale, and deciliter scale fermentation volumes may be used. In addition, larger scale fermentations including fermentations greater than 1 Liter scale can be used. In one embodiment, the fermentation volume will be at or above 1 Liter. In another embodiment, the fermentation volume will be at or above 5 Liters, 10 Liters, 15 Liters, 20 Liters, 25 Liters, 50 Liters, 75 Liters, 100 Liters, 200 Liters, 50 Liters, 1,000 Liters, 2,000 Liters, 5,000 Liters, 10,000 Liters, 50,000 Liters or 100,000 Liters.

In the present disclosure, growth, culturing, and/or fermentation of the transformed host cells is performed within a temperature range permitting survival of the host cells, preferably a temperature within the range of about 4° C. to about 55° C., inclusive.

The ability for a microorganism to produce N-acylglycine surfactants according to this disclosure may be further assayed by isolating and purifying glycine N-acyltransferase proteins to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, column chromatography, immunopurification methods, and others. For example, N-acylglycine surfactants having established molecular adhesion properties can be reversibly fused to a ligand. With the appropriate ligand, the N-acylglycine surfactants can be selectively adsorbed to a purification column and then freed from the column in a relatively pure form. The fused N-acylglycine surfactant is then removed by enzymatic activity. In addition, protein can be purified using immunoaffinity columns or Ni-NTA columns. General techniques are further described in, for example, R. Scopes, Protein Purification: Principles and Practice, Springer-Verlag: N.Y. (1982); Deutscher, Guide to Protein Purification, Academic Press (1990); U.S. Pat. No. 4,511,503; S. Roe, Protein Purification Techniques: A Practical Approach (Practical Approach Series), Oxford Press (2001); D. Bollag, et al., Protein Methods, Wiley-Lisa, Inc. (1996); A K Patra et al., Protein Expr Purif, 18(2): p/ 182-92 (2000); and R. Mukhija, et al., Gene 165(2): p. 303-6 (1995). See also, for example, Ausubel, et al. (1987 and periodic supplements); Deutscher (1990) “Guide to Protein Purification,” Methods in Enzymology vol. 182, and other volumes in this series; Coligan, et al. (1996 and periodic Supplements) Current Protocols in Protein Science Wiley/Greene, NY; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See also, for example, Hochuli (1989) Chemische Industrie 11:69-70; Hochuli (1990) “Purification of Recombinant Proteins with Metal Chelate Absorbent” in Setlow (ed.) Genetic Engineering, Principle and Methods 12:87-98, Plenum Press, NY; and Crowe, et al. (1992) QIAexpress: The High Level Expression & Protein Purification System QIAGEN, Inc., Chatsworth, Calif.

The recombinantly produced and expressed N-acylglycine surfactants can be recovered and purified from the recombinant cell cultures by numerous methods, for example, high performance liquid chromatography (HPLC) can be employed for final purification steps, as necessary.

The molecular weight of a N-acylglycine surfactant can be used to isolate it from cellular debris of greater or lesser size using ultrafiltration through membranes of different pore size (for example, Amicon or Millipore membranes). As a first step, the N-acylglycine surfactant mixture can be ultrafiltered through a membrane with a pore size that has a lower molecular weight cut-off than the molecular weight of the N-acylglycine surfactant. The retentate of the ultrafiltration can then be ultrafiltered against a membrane with a molecular cut off greater than the molecular weight of the N-acylglycine surfactant. The N-acylglycine surfactants will pass through the membrane into the filtrate.

The N-acylglycine surfactants can also be separated from other cellular debris on the basis of its size, net surface charge, hydrophobicity, and affinity for ligands. In addition, the N-acylglycine surfactants can be conjugated to column matrices for isolation. All of these methods are well known in the art. It will be apparent to one of skill that chromatographic techniques can be performed at any scale and using equipment from many different manufacturers (e.g., Pharmacia Biotech).

Upon isolation and purification of N-acylglycine surfactants, the molecules can be used for, but not limited to, personal care.

In the present disclosure, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, topical medicines, and sunscreens.

In the present disclosure, “personal care” is also intended to refer to hair care compositions including, for example, shampoos, leave-on conditioners, styling gels, hairsprays, and mousses. Preferably, the hair care compositions are cosmetically acceptable:

“Personal care” relates to compositions to be topically administered (i.e., not ingested). Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the disclosure may be manufactured by processes well known in the art, for example, by means of conventional mixing, dissolving, granulating, emulsifying, encapsulating, entrapping or lyophilizing processes.

Embodiments of the subject disclosure are further exemplified in the following Examples. It should be understood that these Examples are given by way of illustration only. From the above embodiments and the following Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims. The following is provided by way of illustration and not intended to limit the scope of the invention.

### EXAMPLES

### Example 1

### Identification and Characterization of Glycine-Specific Adenylation Domains

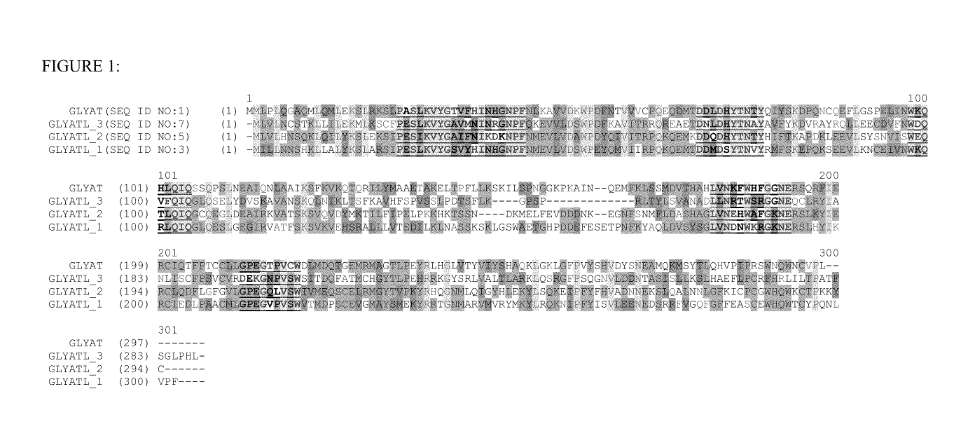

A class of glycine N-acyltransferase proteins are selected from the polypeptides encoded by the following gene sequences of acyl-CoA:glycine N-acyltransferase (GLYAT; NM_005838; SEQ ID NO:1), glycine N-acyltransferase-like 1(GLYATL 1; NM_001220494.2; SEQ ID NO:3), glycine N-acyltransferase-like 2 (GLYATL 2; NM_145016; SEQ ID NO:5), glycine N-acyltransferase-like 3 (GLYATL 3; NM_001010904.1; SEQ ID NO:7). This group of glycine N-acyltransferase proteins were identified and obtained from Genbank (Benson, D., Karsch-Mizrachi, I., Lipman, D. J., Ostell, J., and Wheeler, D. L. (2005). GenBank. Nucleic Acids Res. 33, D34-D38.doi: 10.1093/nar/gki063). Table 1 lists the glycine N-acyltransferase proteins and the aralkyl acyl-CoA:amino acid-N-acyltransferase protein motif domain (that also includes the aralkyl acyl-CoA:amino acid N-acyltransferase, C-terminal region) that were identified from the analysis and search of Genbank. FIG. 1 provides a sequence alignment of the glycine N-acyltransferase protein sequences. Upon completion of an alignment and analysis of the glycine N-acyltransferase sequences, several protein motifs were identified that defined conserved regions that are designated as consensus sequences as diagrammed in FIG. 1. Five consensus sequences were determined to define motifs that are characteristic of glycine N-acyltransferase proteins. Using these motifs to search databases (e.g. GeneBank) one practiced in the art may identify additional putative glycine N-acyltransferase proteins from a variety of organisms. For example the following motif sequences were identified: SEQ ID NO:9—P(A/E)S(L/I)KVYG(T/A/S)(V/I)(F/M/Y)(H/N)I(N/K)(H/R/D)(G/K)NPF; SEQ ID NO: 10—D(D/N)(L/Q/M)D(H/S)YTN(T/A/V)Y; SEQ ID NO:11—W(K/D/E)Q(H/V/T/R)(L/F)QIQ; SEQ ID NO:12—L(V/L)N(K/R/E/D)(F/T/H/N)W(H/S/A/K)(F/R)G(G/K)NE; and, SEQ ID NO: 13—(G/D)(P/E)(E/K)G(T/N/Q/V)(P/L)V(C/S)W. Finally, Table 2 provides the levels of sequence similarity shared between the glycine N-acyltransferase proteins. As is shown in Table 2 the protein sequences shared varying levels of sequence identity ranging from 49.2% to 31.7%. Despite the level of variation in sequence identity, the enzymes perform the same function. The class of glycine N-acyltransferase proteins that are disclosed herein constitute a class of several mammalian specific glycine N-acyltransferase proteins that are categorized as EC:2.3.1.13. Generally, glycine N-acyltransferase proteins that are categorized as EC:2.3.1.13 result in the CoA derivatives of a number of aliphatic and aromatic acids, except that phenylacetyl-CoA or (indol-3-yl)acetyl-CoA cannot act as donor. The enzymes disclosed herein catalyze the conversion of acyl-coA and glycine into coA and N-acylglycine.

TABLE 1
The identified glycine N-acyltransferase and glycine
N-acyltransferase-like proteins.

| | | Protein | DNA | Amino | |
|---|---|---|---|---|---|
| Accession | Gene | SEQ ID | SEQ ID | Acid | |
| Number | Name | NO: | NO: | Specificity | Domain |
| NM_00583 | GLYAT | SEQ ID | SEQ ID | glycine | Aralkyl acyl-CoA: |
| 8 | | NO: 1 | NO: 2 | | amino acid N-acyltransferase |
| | | | | | Aralkyl acyl-CoA: |
| | | | | | amino acid N-acyltransferase, |
| | | | | | C-terminal region |
| NM_00122 | GLYATL | SEQ ID | SEQ ID | glycine | Aralkyl acyl-CoA: |
| 0494.2 | 1 | NO: 3 | NO: 4 | | amino acid N-acyltransferase |
| | | | | | Aralkyl acyl-CoA: |
| | | | | | amino acid N-acyltransferase, |
| | | | | | C-terminal region |
| NM_14501 | GLYATL | SEQ ID | SEQ ID | glycine | Aralkyl acyl-CoA: |
| 6 | 2 | NO: 5 | NO: 6 | | amino acid N-acyltransferase |
| | | | | | Aralkyl acyl-CoA: |
| | | | | | amino acid N-acyltransferase, |
| | | | | | C-terminal region |
| NM_00101 | GLYATL | SEQ ID | SEQ ID | glycine | Aralkyl acyl-CoA: |
| 0904.1 | 3 | NO: 7 | NO: 8 | | amino acid N-acyltransferase |
| | | | | | Aralkyl acyl-CoA: |
| | | | | | amino acid N-acyltransferase, |
| | | | | | C-terminal region |

TABLE 2
The percentage of sequence identity shared
between the glycine N-acyltransferase proteins.

| | GLYAT | GLYATL 1 | GLYATL 2 | GLYATL 3 |
|---|---|---|---|---|
| | (SEQ ID | (SEQ ID | (SEQ ID | (SEQ ID |
| | NO: 1) | NO: 3) | NO: 5) | NO: 7) |
| GLYAT | — | 38.5% | 40.3% | 32.8% |
| (SEQ ID | | | | |
| NO: 1) | | | | |
| GLYATL 1 | 38.5% | — | 49.2% | 31.7% |
| (SEQ ID | | | | |
| NO: 3) | | | | |
| GLYATL 2 | 40.3% | 49.2% | — | 32.3% |
| (SEQ ID | | | | |
| NO: 5) | | | | |
| GLYATL 3 | 32.8% | 31.7% | 32.3% | — |
| (SEQ ID | | | | |
| NO: 7) | | | | |

### Example 2

### Codon Optimization of Native Glycine N-Acyltransferase Gene Sequences

Next, the native coding sequences of Glyat and GlyatL2 were codon optimized for expression in prokaryotic microorganisms. Analysis of the Glyat and GlyatL2 nucleic acid coding sequence revealed the presence of several sequence motifs that were believed to be detrimental to optimal expression, as well as a non-optimal codon composition for expression of the protein. Thus, an achievement of the present disclosure is design of a bacterial optimized gene encoding Glyat and GlyatL2 to generate a DNA sequence that can be optimally expressed in bacterial sp., and in which the sequence modifications do not hinder translation or create mRNA instability.

One may thus use a variety of methods to produce a gene as described herein. An example of one such approach is further illustrated in PCT App. WO 97/13402. Thus, synthetic genes that are functionally equivalent to the Glyat and GlyatL2 gene of the subject disclosure can be used to transform hosts, including bacterial species such as the non-limiting examples of Bacillus subtilis and Escherichia coli. Additional guidance regarding the production of synthetic genes can be found in, for example, U.S. Pat. No. 5,380,831.

To engineer an optimized gene encoding Glyat and GlyatL2 for expression in a bacterial species, a DNA sequence was designed to encode the amino acid sequences utilizing a redundant genetic code established from a codon bias table compiled from the protein coding sequences for the particular host bacterial species. The native Glyat and GlyatL2 polynucleotide sequences were provided to DNA 2.0 (Menlo Park, Calif.) and optimized using the proprietary codon-optimization program available from DNA 2.0.

The newly designed, bacteria optimized Glyat and GlyatL2 polynucleotide sequence is listed in SEQ ID NO: 14 or SEQ ID NO:15, respectively. The resulting DNA sequence has a higher degree of codon diversity for expression in a bacterial microorganism, a desirable base composition, contains strategically placed restriction enzyme recognition sites, and lacks sequences that might interfere with transcription of the gene, or translation of the product mRNA.

Once a bacterial-optimized DNA sequence has been designed on paper or in silico, actual DNA molecules can be synthesized in the laboratory to correspond in sequence precisely to the designed sequence. Such synthetic DNA molecules can be cloned and otherwise manipulated exactly as if they were derived from natural or native sources. Synthesis of DNA fragments comprising SEQ ID NO:14 or SEQ ID NO:15 containing additional sequences such as additional stop codons, 5′ and 3′ restriction sites for cloning, and the addition of a Shine-Delgarno sequence were performed by commercial suppliers. The synthetic DNA was then cloned into expression vectors and transformed into Bacillus subtilis as described in the Examples below.

### Example 3

### Assembly of Glycine N-Acyltransferase Constructs

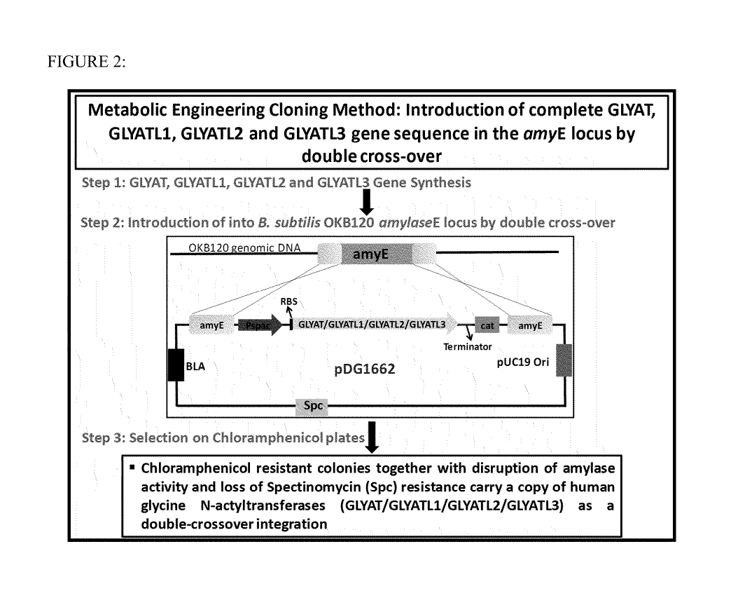

The glycine N-acyltransferase coding sequences were synthesized and assembled under the expression of the inducible promoter, Pspac (SEQ ID NO:16) and ribosome binding sequence (SEQ ID NO:17) and terminated by a termination sequence (SEQ ID NO:18). In addition, the constructs contained native B. subtilis genomic DNA flanking sequences on both ends of the construct. The 5′ end of the gene expression cassette contained the 5′ amyE gene sequence from B. subtilis, and the 3′ end of the gene expression cassette contained the 3′ amyE gene sequence from B. subtilis. The flanking genomic DNA fragments were identical to genomic DNA sequences of the a-amylase gene (amyE) from B. subtilis, and were incorporated into the constructs for integration within the genomic locus. The constructs and flanking genomic DNA were cloned into the pDGI662 plasmid (Bacillus Genetic Stock Center, Biological Sciences 556, 484 W. 12th Ave, Columbus, Ohio 43210-1214). FIG. 2 provides a schematic of the resulting constructs used for transformation (SEQ ID NO: 19—GLYAT expression construct, SEQ ID NO:20—codon optimized GLYAT expression construct, SEQ ID NO:21—GLYATL2 expression construct, and SEQ ID NO:22—codon optimized GLYATL2 expression construct), and a high level overview of the strategy for introducing the glycine N-acyltransferase gene sequences of Glyat and GlyatL2 into the amyE locus of B. subtilis str. OKB120.

### Example 4

### Transformation of Constructs in B. subtilis

The genetic make-up of Bacillus subtilis str. OKB120 is described in detail in; Dirk Vollenbroich, Neena Mehta, Peter Zuber, Joachim Vater, and Roza Maria Kamp (1994). Analysis of Surfactin Synthetase Subunits in srfA Mutants of Bacillus subtilis str. 0KB105. Journal of Bacteriology, Vol. 176, No. 2; p. 395-400. This strain was generated by introducing a transposon mutation in the second module of the surfactin cluster (srfAB) of a surfactin producing strain labeled as OKB105. The resulting mutations to the gDNA of the strain are labeled as B. subtilis str. OKB120 (pheA1 sfp srfA::Tn917). The presence of this transposon insertion mutation renders the strain OKB120 incapable of producing the native surfactin product. However, the strain is capable of producing tetrapeptide and shorter Srf fragments including acyl-glutamate. Accordingly, the strain was transformed with the above described plasmids using the protocol as described in Guerout-Fleury, A. M., Frandsen, N. and Stragier, P. (1996) Plasmids for ectopic integration in Bacillus subtilis. Gene 180 (1-2), 57-61.

### Example 5

### Molecular Confirmation of Genomic DNA Integration of the GLYAT and GLYATL2 Construct within the B. subtilis Genome

After the separate transformation of each gene construct into the B. subtilis chromosome, molecular confirmation assays were completed to confirm the integration of the Glyat and GlyatL2 gene sequence into the α-amylase gene (amyE) locus of the genome by homologous recombination. Integration of the Glyat and GlyatL2 gene construct within the amyE genomic locus resulted in the subsequent disruption of the amyE gene function. Accordingly, colony PCR was employed to detect the successful delivery of the glycine N-acyltransferase gene constructs within the bacterial chromosome. Table 3 lists the PCR primers used for colony PCR validation to confirm the presence of Glyat and GlyatL2 constructs and the corresponding gene sequences within the genome of B. subtilis. In addition, the disruption of the amyE locus was validated by assaying amylase production on starch containing plates (Guerout-Fleury, A. M., Frandsen, N. and Stragier, P. (1996) Plasmids for ectopic integration in Bacillus subtilis, Gene 180 (1-2), 57-61). Furthermore, transformants were screened for the loss of spectinomycin resistance, which indicates that a double crossover event had occurred. B. subtilis str. OKB120 strains containing the glycine N-acyltransferase genes were obtained for each of the above described constructs and were fermented to produce N-acylglycine.

TABLE 3
The gene sequence information for glycine
N-acyltransferase gene constructs in this
study and the PCR validation primers used
in this sturdy.

| Strain/ | |
|---|---|
| Construct | PCR Validation Primers |
| Glyat | (SEQ ID NO: 23) |
| | F-TTCTGTTTCTGCTTCGGTATGT |
| | (SEQ ID NO: 24) |
| | R-GAGGCTTACTTGTCTGCTTTCT |
| GlyatL2 | (SEQ ID NO: 25) |
| | F-TTCTGTTTCTGCTTCGGTATGT |
| | (SEQ ID NO: 26) |
| | R-GAGGCTTACTTGTCTGCTTTCT |

### Example 6

### Fermentation of N-Acylglycine

Identified bacterial colonies that contained the genomic integrant of the glycine N-acyltransferase gene sequences were isolated and cultured in a defined minimal medium with or without the addition of 50 mM glycine (Media C Recipes for Surfactin production in Bacillus subtilis. Bacterial production of antimicrobial biosurfactants by Bacillus subtilis, Keenan Bence Thesis presented in partial fulfillment of the requirements for the Degree of Master of Science in engineering (chemical engineering) in the Faculty of Engineering at Stellenbosch University, Supervisor Prof. K. G. Clarke December 2011). The cultures in the shake flask format (30 ml) were grown to OD₆₀₀˜0.8 before induction of the Pspac promoter by addition of 1 mM IPTG into the growth medium. The fermentation was completed at a temperature for optimal B. subtilis growth and at a volume of from about 10 ml to 10 L. The fermentation medium was centrifuged and cell extracts were prepared at 20, 48 and 72 hours using a 3:1 ratio of methanol to whole broth. The cell extracts were concentrated 2.5× in a Speedvac™ and dissolved in methanol for analysis of the presence of the novel product N-acylglycines by LC/MS.

### Example 7

### Heterologous Expression of GLYAT and GLYATL2 in Escherichia coli

The expression of GLYAT and GLYATL2 for recruitment of glycine into a medium chain-length β-hydroxy fatty acid peptide chain, in vivo, to subsequently produce N-acylglycines was tested in an Escherichia coli heterologous expression system. The genes encoding both the GLYAT and GLYATL2 proteins were assembled into vector constructs and expressed separately into E. coli cells. Ultimately, the protein products of GLYAT and GLYATL2 as well as N-acylglycines were isolated from the cultures.

A vector construct containing the Glyat gene was constructed. Minor modifications were made to the Glyat gene such as the first Methionine was removed and an additional twenty-one codons were added to the N-terminus of the coding sequence. The variant sequence is provided as SEQ ID NO: 27 for the protein and SEQ ID NO:28 for gene of Glyat. The modified Glyat gene was chemically synthesized and cloned into the pETDuet-1 vector (EMD Biosciences) by Synthetic Genomics Inc (San Diego, Calif.).

Likewise, a vector construct containing the GlyatL2 gene was constructed. Minor modifications were made to the GlyatL2 gene such as the first Methionine was removed and an additional twenty-one codons were added to the N-terminus of the coding sequence. The variant sequence is provided as SEQ ID NO: 29 for the protein and SEQ ID NO:30 for the gene of GlyatL2. The modified GlyatL2 gene was chemically synthesized and cloned into the pETDuet-1 vector (EMD Biosciences) by Synthetic Genomics Inc (San Diego, Calif.).

TABLE 4
The pET-Duet expression vectors used for over-expression of
the Glyat and GlyatL2 genes in E. coli.

| | Selection | | |
|---|---|---|---|
| E. coli expression vector | marker | Gene | SEQ ID NO |
| pHis-GLYAT-pETDuet-1 | Ampicillin | Glyat | SEQ ID NO: 31 |
| pHis-GLYATL2-pETDuet-1 | Ampicillin | GlyatL2 | SEQ ID NO: 2 |

### Example 8

### Transformation and Expression of GLYAT and GLYATL2 in Escherichia coli

The E. coli heterologous expression studies were conducted using the competent BL21 (DE3) cells acquired from EMD Biosciences. Transformations were performed as per the kit instructions and involved mixing a 50 μL aliquot of competent cells with 1 μL of the vector.

The E. coli transformants were selected on LB agar plates containing 100 μg/ml of ampicillin. The plates were incubated at 37° C. for 16 hours. A starter culture was started by transferring a single colony of transformant into 50 mL of LB medium containing 100 μg/ml of ampicillin and incubated at 37° C. with shaking at 220 rpm for overnight. The next day, 7 ml of starter culture was inoculated into 800 ml of Terrific Broth and the culture was incubated at 37° C. until the culture reached an optical density (OD₆₀₀ₙₘ) of 0.5. Then IPTG at a final concentration of 1 mM was added to induce the expression of the Glyat or GlyatL2 genes and the culture was transferred to a 15° C. incubator for 16 hours. At the end of 16 hours, the culture was centrifuged at 8,000 rpm to pellet the cells. The cell pellet was divided into two aliquots and stored at −80° C. overnight before purification.

Next, the E. coli cell pellet from the over-expression of 400 ml of culture was suspended in B-PER reagent (Pierce; Rockford, Ill.) containing 1 μg/ml of DNAse, 1 μg/ml of lysozyme, 1 mM DTT, and protease inhibitor cocktail. The suspension was rocked gently for 30 minutes at room temperature and centrifuged at 15,000×g for 20 minutes. The supernatant was separated and incubated with 5 ml of Co-NTA resin that had been pre-equilibrated with an equilibration buffer (50 mM sodium phosphate pH 8.0 containing 300 mM sodium chloride, 20 mM imidazole, 50 μL protease inhibitor cocktail and 15% glycerol). Following an incubation period of 1 hour at 4° C., the GLYAT and GLYATL2 bound resin was washed with 5 volumes of equilibration buffer. The GLYAT and GLYATL2 were eluted from the Co-NTA resin with equilibration buffer containing 200 mM imidazole. The eluted proteins were dialyzed against Phosphate Buffer Solution and stored as a 20% glycerol solution at −20° C.

### Example 9

### Quantitation and Structure Validation of N-Acylglycine Products

Metabolites in extracts prepared as described above were analyzed by three methods: A, B and/or C. Selected metabolites were quantified by Method A, with separation using UHPLC followed by quantitation using selected ion monitoring (SIM)-mass spectrometry (MS). Identities of metabolites were validated by separation using UHPLC employing one of two separation methods; Method B for B. subtilis metabolites or Method C for E. coli metabolites, followed by high resolution MS and MS/MS, as described below. The LC-SIM-MS analysis system comprised the following components: G4220A Infinity 1290 binary pump, G4226A Infinity 1290 autosampler, G4212A Infinity 1290 diode array detector with 10 mm path length flow cell (G4212-60008), G1316C thermostated column compartment (TCC) and G6140A single quadrupole mass spectrometer running under Agilent ChemStation (version B.04.02 SPI [212]). The system was mass calibrated each day of use using the Agilent CheckTune and/or Autotune routines. Operating parameters were as follows: temperature 350° C., nitrogen drying gas flow: 12 L/min; nebulization pressure: 35 psi; capillary voltage: 3000V, fragmentor voltage: 70V. The LC-accurate MS/MS (QTOF-MS) analysis system comprised the following components: Agilent G4220A Infinity 1290 binary pump, HTC-XT Leap-PAL autosampler, G4212A Infinity 1290 diode array detector with 60 mm path length flow cell (G4212-60007), G1316C column compartment at room temperature (approx. 25° C.) and AB Sciex 5600 quadrupole/time of flight (QTOF-MS) mass spectrometer running under Analyst TF software V 1.6, with data interrogation using Peakview V 1.2. The mass spectrometer was calibrated using a commercial APCI negative calibration solution for the AB Sciex system in the negative ionization mode. Mass measurements on eluted metabolites were made using the QTOF-MS instrument for mass spectra, measured to +/−0.001 Da accuracy, for example m/z 300.001+/−0.001 Da. Operating parameters were as follows: full-scan range 100-1000 Da (for UHPLC Method B) or 100-2000 Da (for UHPLC Method C), MS/MS scan range: 100-1000 Da; accumulation time: full-scan 0.15 sec; MS/MS: 0.10 sec; temperature 450-500° C.; ionspray floating voltage: 4500-5500; declustering potential: 80-100; scan event 1: TOF MS full scan collision energy 5-10 eV; scan events 2-4: product ion IDA collision energy 20-35 eV with a spread of 15 eV. MS/MS spectra were acquired using the following targeted inclusion lists, corresponding to [M-H]⁻ for each targeted compound: Method B: m/z 300.2, 314.2, 356.2, 372.2 and 386.2; Method C: m/z 178.0, 130.0, 158.0, 200.1, 214.1, 228.1, 242.1, 256.1, 254.1, 270.2, 286.2, 268.1, 284.2, 227.2, 300.2, 243.1, 282.2, 225.1, 298.2, 314.2, 296.2, 312.2, 310.2, 326.2, 324.2, 340.2 and 338.2.

For Methods A and B, metabolites in extracts were separated using an Agilent Eclipse Plus C18 (100×3.0 mm; 1.8 m particle size) column eluted at 0.425 mL/min with a gradient of water-formic acid (99.9:0.1 v/v; “A”) and acetonitrile-formic acid (99.9:0.1 v/v; “B”). The gradient was as follows: 0-1.33 min: A:B=50:50; 1.33-13.33 min linear gradient to A:B=0:100; 13.33-14.67 min hold at A:B=0:100; 14.67-16.00 min linear gradient to A:B=50:50 and hold to 17.33 min.

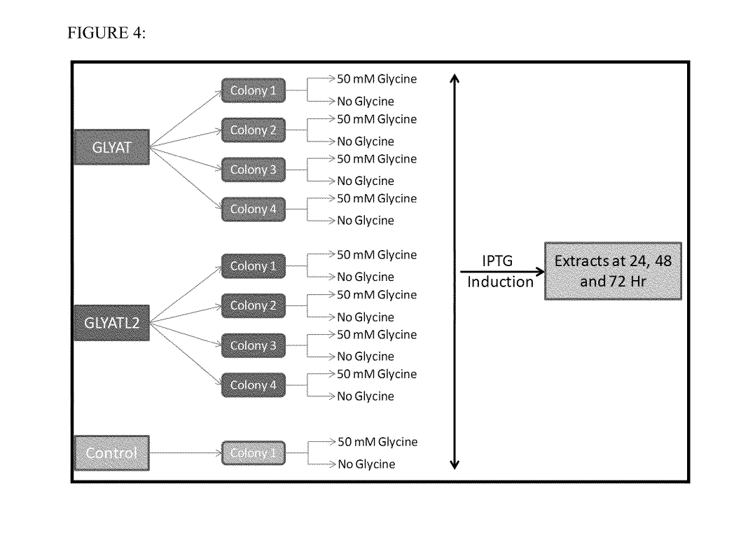

For Method C, metabolites in extracts were separated using an Agilent Eclipse Plus C18 (150×3.0 mm; 1.8 μm particle size) column eluted at 0.5 mL/min with a gradient of solvents “A” and “B”. The gradient was as follows: 0-2 min: A:B=90:10; 2-26 min linear gradient to A:B=4:96; 26-40 min hold at A:B=4:96; 40-41 min linear gradient to A:B=90:10 and hold to 45 min. Injection sizes were 2 μL for standards mixtures and 20 μL for fermentation extracts. The design of the experiments and the fermentations that were analyzed via these protocols are shown in FIG. 4.

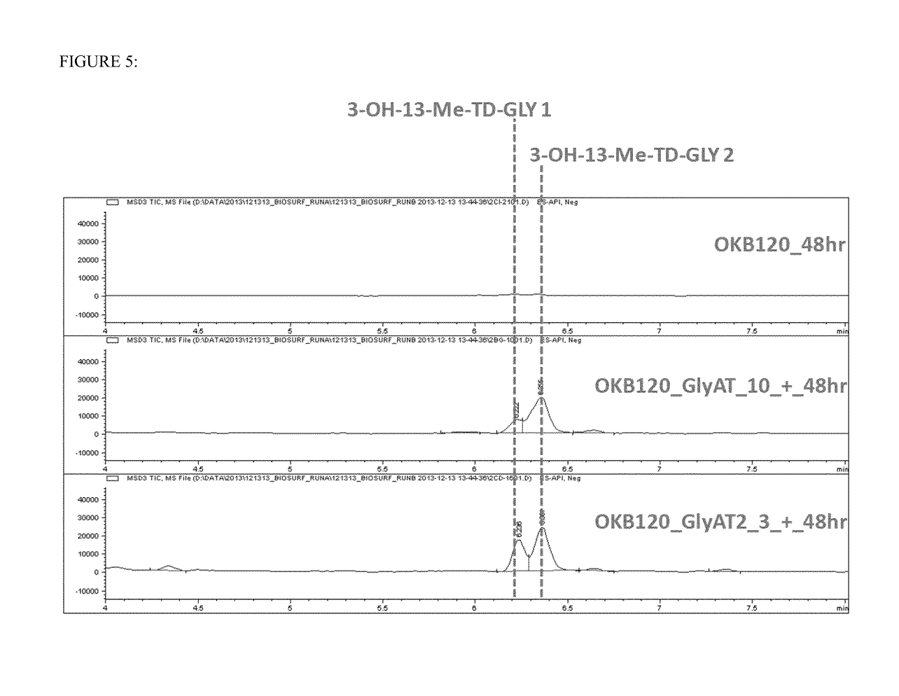

The novel products (1) and (2), shown in FIG. 3, were detected in engineered strains of B. subtilis by acquiring a selected ion chromatogram at m/z 314.2, and quantitation was performed with a multi-level calibration curve in external standard mode using authentic 3-OH—C14-GLY compound (3) (range: 0.001 to 10.136 μg/mL; Matreya LLC Lipids and Biochemicals, Pleasant Gap; PA 16823), which was detected by acquiring a selected ion chromatogram at m/z 300.2. Example chromatograms from the application of this method to extracts of the engineered strains, are shown in FIG. 5 (strains GLYAT-10+ and GLYATL2-3+), which demonstrated successful production of these novel compounds in both of these constructs. Two chromatographic peaks with the same accurate mass (see below) were observed in the B. subtilis strains during the SIM-MS assay. These peaks were concluded to be isomers of 3-OH—C15-GLY, most likely methyl group positional isomers in the fatty acid chain. In comparison, these two product peaks were not detected in the control (non-engineered) B. subtilis str. OKB120 strains. These data gave a quantitative estimate for combined production levels of products (1) and (2) in the range 3-10 μg/L broth (FIG. 5).

A summary of the UHPLC and mass spectral data supporting the structures of the compounds in FIG. 3 produced by B. subtilis strains GLYAT-10 and GLYATL2-3 appears in Table 5. While no authentic standards of the methyl-group isomers 3-OH—C15-GLY products (1) or (2) were available for comparison, detection of two compounds having the anticipated [M-H]⁻ ion at m/z 314.234, which eluted closely following an authentic standard of 3-OH—C14-GLY compound (3), supports the production of the target molecule since an additional methyl group would increase the lipophilicity of the molecule relative to 3-OH—C14-GLY compound (3), thereby causing it to adsorb slightly more strongly to the UHPLC analysis stationary phase, and elute slightly later. The measured weights for the parent ion in each case showed good agreement with the theoretical values, validating the proposed structures.

TABLE 5
Summary of HPLC retention times and high resolution mass spectral data for novel metabolites (1 and 2 as shown in FIG. 3)
formed in engineered strains of B. subtilis and an authentic standard of the analog 3-OH—C14-GLY (3).

| | | | | | Proposed | | | |
|---|---|---|---|---|---|---|---|---|
| | | LC RT | LC RT | Proposed | Molecular | Theoretical | Measured | Measured Mass |
| Sample Type | Compound | (Extract) | (Standard) | Ion¹ | Formula | Mass | Mass (Extract) | (Standard) |
| Authentic | 3-OH—C14-GLY | | 5.43 | parent | C₁₆H₃₀NO₄⁻ | 300.218 | | 300.218 |
| Standard | (3) | | | | | | | |
| GLYAT-10 | 3-OH—C15-GLY | 6.36 | | parent | C₁₇H₃₂NO₄⁻ | 314.234 | 314.234 | |
| extract | isomer 1 (1) | | | | | | | |
| | 3-OH—C15-GLY | 6.50 | | parent | C₁₇H₃₂NO₄⁻ | 314.234 | 314.235 | |
| | isomer 2 (2) | | | | | | | |
| GLYAT-10 | 3-OH—C15-GLY | 6.39 | | parent | C₁₇H₃₂NO₄⁻ | 314.234 | 314.236 | |
| extract² | isomer 1 (1) | | | | | | | |
| | 3-OH—C15-GLY | 6.54 | | parent | C₁₇H₃₂NO₄⁻ | 314.234 | 314.237 | |
| | isomer 2 (2) | | | | | | | |
| GLYATL2-3 | 3-OH—C15-GLY | 6.35 | | parent | C₁₇H₃₂NO₄⁻ | 314.234 | 314.233 | |
| extract | isomer 1 (1) | | | | | | | |
| | 3-OH—C15-GLY | 6.50 | | parent | C₁₇H₃₂NO₄⁻ | 314.234 | 314.233 | |
| | isomer 2 (2) | | | | | | | |
| GLYATL2-3 | 3-OH—C15-GLY | 6.37 | | parent | C₁₇H₃₂NO₄⁻ | 314.234 | 314.234 | |
| extract² | isomer 1 (1) | | | | | | | |
| | 3-OH—C15-GLY | 6.50 | | parent | C₁₇H₃₂NO₄⁻ | 314.234 | 314.233 | |
| | isomer 2 (2) | | | | | | | |

¹All parent ions represent [M − H]⁻
²Supplemented with exogenous glycine

A summary of the UHPLC and mass spectral data supporting the structures of the compounds in FIG. 3 produced by E. coli strain transformed with GLYATL2 appears in Table 6. The UHPLC retention times and accurate parent ion and fragment mass spectral data matched those for authentic standards for compounds 3-10 of FIG. 3 except C18-GLY (9), for which no authentic standard was available. These data, therefore, validated the proposed product structures.

TABLE 6
Summary of UHPLC retention times and high resolution mass spectral data for metabolites produced in E. coli transformed with
GLYATL2, and authentic standards.

| | | | | Proposed | | | |
|---|---|---|---|---|---|---|---|
| | UHPLC RT | UHPLC RT | | Molecular | Theoretical | Measured Mass | Measured Mass |
| Compound | (Extract) | (Standard) | Proposed Ion¹ | Formula | Mass | (Extract) | (Standard) |
| C8-GLY (4) | 12.10 | 12.11 | parent | C₁₀H₁₈NO₃⁻ | 200.129 | 200.129 | 200.129 |
| | | | M-CO₂ | C₉H₁₈NO⁻ | 156.139 | 156.141 | NO² |
| | | | M-CH₂CO₂ | C₈H₁₆NO⁻ | 142.124 | 142.122 | NO |
| | | | M-H₂—NHCH₂CO₂ | C₈H₁₃NO⁻ | 125.097 | 125.100 | NO |
| C10-GLY (5) | 15.48 | 15.48 | parent | C₁₂H₂₂NO₃⁻ | 228.161 | 228.161 | 228.162 |
| | | | M-H₂O | C₁₂H₂₀NO₂⁻ | 210.150 | 210.151 | NO |
| | | | M-CO₂ | C₁₁H₂₂NO⁻ | 184.171 | 184.170 | 184.171 |
| | | | M-H₂—CO₂ | C₁₁H₂₀NO⁻ | 182.155 | 182.157 | 182.157 |
| | | | M-NHCH₂CO₂ | C₁₀H₁₉O⁻ | 155.144 | 155.141 | NO |
| | | | M-H₂—NHCH₂CO₂ | C₁₀H₁₇O⁻ | 153.129 | NO | 153.129 |
| C12-GLY (6) | 18.63 | 18.63 | parent | C₁₄H₂₆NO₃⁻ | 256.192 | 256.193 | 256.193 |
| | | | M-H₂O | C₁₄H₂₄NO₂⁻ | 238.181 | NO | 238.181 |
| | | | M-CO₂ | C₁₃H₂₆NO⁻ | 212.202 | 212.202 | 212.201 |
| | | | M-H₂—CO₂ | C₁₃H₂₄NO⁻ | 210.186 | 210.186 | 210.185 |
| | | | M-NHCH₂CO₂ | C₁₂H₂₃O⁻ | 183.175 | 183.178 | 183.178 |
| | | | M-H₂—NHCH₂CO₂ | C₁₂H₂₁O⁻ | 181.160 | 181.152 | 181.158 |
| C14-GLY (7) | 21.75 | 21.76 | parent | C₁₆H₃₀NO₃⁻ | 284.223 | 284.223 | 284.223 |
| | | | M-CO₂ | C₁₅H₃₀NO⁻ | 240.233 | 240.234 | 240.223 |
| | | | M-H₂—CO₂ | C₁₅H₂₈NO⁻ | 238.218 | 238.221 | 238.219 |
| | | | M-CH₂CO₂—CH₂ | C₁₃H₂₆NO⁻ | 212.202 | 212.201 | NO |
| | | | M-NHCH₂CO₂ | C₁₄H₂₇O⁻ | 211.207 | 211.203 | 211.205 |
| | | | M-H₂—NHCH₂CO₂ | C₁₄H₂₅O⁻ | 209.191 | 209.192 | 209.190 |
| C16-GLY (8) | 24.74 | 24.77 | parent | C₁₈H₃₄NO₃⁻ | 312.254 | 312.255 | 312.255 |
| | | | M-H₂O | C₁₈H₃₂NO₂⁻ | 294.244 | NO | 294.242 |
| | | | M-CO₂ | C₁₇H₃₄NO⁻ | 268.265 | 268.265 | 268.265 |
| | | | M-H₂—CO₂ | C₁₇H₃₂NO⁻ | 266.249 | 266.251 | 266.252 |
| | | | M-NHCH₂CO₂ | C₁₆H₃₁O⁻ | 239.238 | 239.240 | NO |
| | | | M-H₂—NHCH₂CO₂ | C₁₆H₂₉O⁻ | 237.222 | 237.222 | 237.222 |
| C18-GLY (9) | 27.39¹ | NA³ | parent | C₂₀H₃₈NO₃⁻ | 340.286 | 340.286 | NA |
| | | | M-CO₂ | C₁₉H₃₈NO⁻ | 296.296 | 296.295 | NA |
| | | | M-H₂—CO₂ | C₁₉H₃₆NO⁻ | 294.281 | 294.281 | NA |
| | | | M-H₂—NHCH₂CO₂ | C₁₈H₃₃NO⁻ | 265.254 | 265.253 | NA |
| 3-OH—C14- | 18.35 | 18.35 | parent | C₁₆H₃₀NO₄⁻ | 300.218 | 300.218 | 300.220 |
| GLY (3) | | | | | | | |
| | | | M-H₂O | C₁₆H₂₈NO₃⁻ | 282.208 | NO | 282.207 |
| | | | M-CO₂—H₂O | C₁₅H₂₈NO⁻ | 238.218 | 238.222 | 238.218 |
| | | | M-C₅H₉NO₃ | C₁₁H₂₁O⁻ | 169.160 | 169.160 | NO |
| | | | M-C₁₂H₂₄O | C₄H₆NO₃⁻ | 116.035 | NO | 116.034 |
| 3-OH—C14 | 21.03 | 21.04 | parent | C₁₄H₂₇O₃⁻ | 243.197 | 243.197 | 243.197 |
| (10) | | | M-H₂—CO₂ | C₁₃H₂₅O⁻ | 197.191 | 197.188 | 197.191 |
| | | | M-H₂—CH₂CO₂ | C₁₂H₂₃O⁻ | 183.175 | 183.170 | 183.178 |
| | | | M-CH₂CO₂H—CH₃ | C₁₁H₂₁O⁻ | 169.160 | 169.162 | 169.161 |
| | | | M-CH₂CO₂H—C₂H₅ | C₁₀H₁₉O⁻ | 155.144 | 155.139 | NO |

¹All parent ions represent [M − H]⁻
²This fragment was not observed
³Standard of this compound was not available

In conclusion, LC/MS results demonstrate that a microorganisms like B. subtilis str. OKB120 and E. coli strains expressing the GLYAT and GLYATL2 proteins can successfully recruit glycine into a medium chain-length β-hydroxy fatty acid peptide chain, in vivo, resulting in the desired production of N-acylglycine.

While aspects of this invention have been described in certain embodiments, they can be further modified within the spirit and scope of this disclosure. This application is therefore intended to cover any variations, uses, or adaptations of embodiments of the invention using its general principles. Further, this application is intended to cover such departures from the present disclosure as come within known or customary practice in the art to which these embodiments pertain and which fall within the limits of the appended claims.

### Example 10

### Synthesis of N-(3-hydroxytetradecanoyl)glycine

The compound N-(3-hydroxytetradecanoyl)glycine can be prepared by a five-step procedure that is outlined in the instant example, as well as the experimental details that follow.

In summary, lauric aldehyde is treated with allymagnesium chloride in THF to yield 4-hydroxy-1-pentadecene (1). The hydroxyl group of compound 1 is converted to the acetate ester via acetic anhydride/pyridine to yield 4-acetoxy-1-pentadecene (2). The terminal double bond in compound 2 is oxidized using sodium periodate in the presence of ruthenium (III) chloride monohydrate to yield 3-acetoxytetradecanoic acid (3). Carboxylic acid 3 is converted to the corresponding acid chloride in situ, which is then treated with an excess of glycine methyl ester hydrochloride in the presence of pyridine to yield N-(3-acetoxytetradecanoyl)glycine methyl ester (4), which was not isolated but carried on to the next step. Hydrolysis of the acetate and methyl ester functionalities in 4 is carried out by treatment with sodium hydroxide in water to yield the final product, N-(3-hydroxytetradecanoyl)glycine (5).

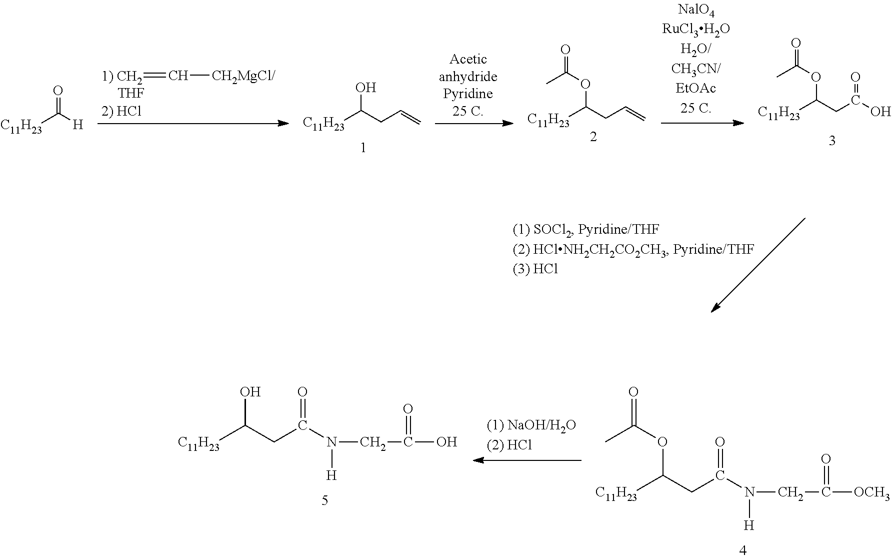

### Synthesis of 4-hydroxy-1-pentadecene (1)

To a dry 500 mL 3-neck round bottom flask fitted with a stir bar, condenser, and 250 mL addition funnel was added allylmagnesium chloride (113 mL of 2.0 M, 0.0.226 mol) via canula to an addition funnel. To the addition funnel was then added dry THF (250 mL) via canula. To the addition funnel was next added dry THF (60 mL) and lauric aldehyde (40.0 mL, 33.2 g, 0.180 mol). The Grignard/THF solution in the flask was cooled to 0° C., then the aldehyde solution was added dropwise over 45 minutes. Once addition had been completed, the flask was allowed to warm to room temperature over a one hour period. The reaction mixture was then cooled to 0° C. and the excess Grignard reagent was quenched by the dropwise addition of isopropanol (60 mL). The reaction mixture was stirred for 1 h, then solvent was removed under reduced pressure to yield a white solid. Methylene chloride (300 mL) and 5% hydrochloric acid (300 mL) were added to the white solid and shaken until the contents dissolved to form two layers. The lower organic layer was separated, dried over magnesium sulfate, filtered, then solvent was removed under reduced pressure to yield a clear oil. The oil was dried under vacuum for 16 hours. Yield: 39.51 g (96.8%). The ¹H and ¹³C NMR spectra were consistent with the structure of 4-hydroxy-1-pentadecene (1).

### Synthesis of 4-acetoxy-1-pentadecene (2)

To a 1 L round bottom flask containing 4-hydroxy-1-pentadecene (1) (39.51 g, 0.175 mol) was added pyridine (150 mL, 147 g, 1.8 mol). Acetic anhydride (170 mL, 184 g, 1.80 mol) was added dropwise to the pyridine solution over a 30 minute period. On completion of addition, the solution was stirred 3 hours. The reaction mixture was then poured into 5% HCl (1500 mL), stirred for 2 hours, then extracted with methylene chloride (2×300 mL). Solvent was removed under reduced pressure to yield an oil. The oil was stirred in water (400 mL) for 2 hours to hydrolyze any residual acetic anhydride. Crude 2 was extracted from the aqueous/oil mixture with methylene chloride (2×300 mL). The methylene chloride solution of 2 was dried over magnesium sulfate, filtered, and solvent was removed under reduced pressure to yield an oil, which was dried under vacuum for 16 hours. Yield 40.4 g (86%). The ¹H and ¹³C NMR spectra were consistent with the structure of 4-acetoxy-1-pentadecene (2).

### Synthesis of 3-acetoxytetradecanoic acid (3)

To a 250 mL round bottom flask containing a stir bar and fitted with a condenser and addition funnel was added 4-acetoxy-1-pentadecene (2) (2.68 g, 10.0 mmol), RuCl₃.H₂O (0.0225 g, 0.100 mmol), ethyl acetate (20 mL), and acetonitrile (20 mL). NaIO4 (10.69 g, 50 mmol) was dissolved in water (100 mL total solution) and was added to the addition funnel. The contents of the flask were heated to 50° C. and the sodium periodate solution was added dropwise over 45 minutes. On completion of addition, the reaction mixture (clear solution with dark insoluble oil) was stirred at 50° C. for 3 h to yield a milky reaction mixture. The reaction mixture was cooled to room temperature and poured into water (150 mL). The organic components were extracted with methylene chloride (2×100 mL) during which time the color of the organic phase changed from yellow to black. The organic layer was washed with 5% HCl (100 mL), separated, dried over magnesium sulfate and filtered, yielding a gray-colored filtrate. Solvent was reduced under reduced pressure to yield a dark oil. The oil was dried under vacuum for 16 hours yielding a dark-colored solid. Yield: 2.61 g (91.1%). The ¹H and ¹³C NMR spectra were consistent with the structure of 3-acetoxy-1-tetradecanoic acid (3).

### N-(3-hydroxytetradecanoyl)glycine (5)

To a 500 mL Erlenmeyer flask containing a stir bar was added 3-acetoxytetradecanoic acid (3) (40.12 g, 140.00 mmol), pyridine (22.16 g, 280.0 mmol), and THF (140 mL). Thionyl chloride (33.32 g, 280.0 mmol) was then added dropwise. The reaction mixture was stirred for 1 hour, then was added to a stirred mixture of glycine methyl ester hydrochloride (70.28 g, 560.0 mmol) and pyridine (88.60 g, 1120.0 mmol) in THF (280 mL). After stirring for 1 hour, the reaction mixture was acidified with concentrated HCl and was poured into a separatory funnel containing methylene chloride (500 mL) and water (500 mL). After shaking and separation of the organic layer, solvent was removed under reduced pressure to yield a dark oil. To the oil was added a solution of sodium hydroxide (10.8 g, 270 mmol) in water (500 mL), which was then heated to 80-90 C for 1 hour. The reaction mixture was cooled to 50° C., then acidified with concentrated HCl to about pH 6, causing an amber-colored solid to precipitate. An equal volume of ethyl acetate was added to the flask and heated to boiling. The organic layer was separated, then solvent was removed under reduced pressure to yield a dark solid. Methylene chloride (300 mL) was added and the mixture was stirred until all or the dark material was either dissolved or was suspended in the methylene chloride. Collected the insoluble material by suction filtration, washed with methylene chloride, and air dried to yield an off-white solid. Dried under vacuum for 16 hours. Yield: 12.8 g (30.3%). The ¹H and ¹³C NMR spectra were consistent with the structure of N-(3-hydroxytetradecanoyl)glycine (5).

SEQUENCE LISTING
SEQ ID NO: 1 - GLYAT-Protein Sequence
mmlplqgaqmlqmlekslrkslpaslkvygtvfhinhgnpfnlkavvdkwpdfntvvvcpqeqdmtddldhytntyqiyskdpq
ncqeflgspelinwkqhlqiqssqpslneaiqnlaaiksfkvkqtqrilymaaetakeltpfllkskilspnggkpkainqemfklssmd
vthahlvnkfwhfggnersqrfierciqtfptccllgpegtpvcwdlmdqtgemrmagtlpeyrlhglvtyviyshaqklgklgfpvys
hvdysneamqkmsytlqhvpiprswnqwncvpl
SEQ ID NO: 2 - GLYAT-Nucleotide Sequence
atgatgttaccattgcaaggtgcccagatgctgcagatgctggagaaatccttgaggaagagcctcccagcatccttaaaggtttatggaact
gtctttcacataaaccacggaaatccattcaatctgaaggctgtggtggacaagtggcctgattttaatacagtggttgtctgccctcaggagc
aggatatgacagatgaccttgatcactataccaatacttaccaaatctactccaaagatccccaaaactgtcaggaattccttggatcaccaga
actcatcaactggaaacagcatttacagattcaaagttcacagcctagcctgaatgaggctatacaaaatcttgcagccattaagtccttcaaa
gtcaaacaaacacaacgcattctctatatggcagctgaaacagccaaggaactgactcctttcctgctgaaatcaaagattttatctcccaatg
gtggcaaacccaaggccatcaaccaagagatgtttaaactctcatctatggatgttacccatgctcacttggtgaataaattctggcattttggt
ggtaatgagaggagccagagattcattgagcgctgcattcagacctttcccacctgctgtctcctggggcctgaggggacccctgtgtgctg
ggatctaatggaccagactggagagatgagaatggcaggcaccttgccggaataccggctccacggccttgtgacgtatgtcatctattccc
acgcccagaaattgggcaaacttgggtttcctgtctattctcatgtagactacagcaatgaagctatgcaaaaaatgagttacacactgcaaca
tgttcccattcccagaagctggaaccagtggaactgtgtacctctgtga
SEQ ID NO: 3 GLYATL1-Protein Sequence
MILLNNSHKLLALYKSLARSIPESLKVYGSVYHINHGNPFNMEVLVDSWPEYQMVIIRPQ
KQEMTDDMDSYTNVYRMFSKEPQKSEEVLKNCEIVNWKQRLQIQGLQESLGEGIRVAT
FSKSVKVEHSRALLLVTEDILKLNASSKSKLGSWAETGHPDDEFESETPNFKYAQLDVS
YSGLVNDNWKRGKNERSLHYIKRCIEDLPAACMLGPEGVPVSWVTMDPSCEVGMAYS
MEKYRRTGNMARVMVRYMKYLRQKNIPFYISVLEENEDSRRFVGQFGFFEASCEWHQ
WTCYPQNLVPF
SEQ ID NO: 4 GLYATL1 - Nucleotide Sequence
ATGATCCTACTGAATAACTCCCATAAGCTGCTGGCCCTATACAAATCCTTGGCCAGGA
GCATCCCTGAGTCCCTGAAGGTGTATGGCTCTGTGTATCACATCAATCACGGGAACCC
CTTCAACATGGAGGTGCTGGTGGATTCCTGGCCTGAATATCAGATGGTTATTATCCGG
CCTCAAAAGCAGGAGATGACTGATGACATGGATTCATACACAAACGTATATCGTATGT
TCTCCAAAGAGCCTCAAAAATCAGAAGAAGTTTTGAAAAATTGTGAGATCGTAAACT
GGAAACAGAGACTCCAAATCCAAGGTCTTCAAGAAAGTTTAGGTGAGGGGATAAGAG
TGGCTACATTTTCAAAGTCAGTGAAAGTAGAGCATTCGAGAGCACTCCTCTTGGTTAC
GGAAGATATTCTGAAGCTCAATGCCTCCAGTAAAAGCAAGCTTGGAAGCTGGGCTGA
GACAGGCCACCCAGATGATGAATTTGAAAGTGAAACTCCCAACTTTAAGTATGCCCA
GCTGGATGTCTCTTATTCTGGGCTGGTAAATGACAACTGGAAGCGAGGGAAGAATGA
GAGGAGCCTGCATTACATCAAGCGCTGCATAGAAGACCTGCCAGCAGCCTGTATGCTC
GGCCCAGAGGGAGTCCCGGTCTCATGGGTAACCATGGACCCTTCTTGTGAAGTAGGA
ATGGCCTACAGCATGGAAAAATACCGAAGGACAGGCAACATGGCACGAGTGATGGTG
CGATACATGAAATATCTGCGTCAGAAGAATATTCCATTTTACATCTCTGTGTTGGAAG
AAAATGAAGACTCCCGCAGATTTGTGGGGCAGTTTGGTTTCTTTGAGGCCTCCTGTGA
GTGGCACCAATGGACTTGCTACCCACAGAATCTAGTTCCATTTTAG
SEQ ID NO: 5 - GLYATL2-Protein Sequence
mlvlhnsqklqilyksleksipesikvygaifnikdknpfnmevlvdawpdyqivitrpqkqemkddqdhytntyhiftkapdklee
vlsysnvisweqtlqiqgcqegldeairkvatsksvqvdymktilfipelpkkhktssndkmelfevdddnkegnfsnmfldashagl
vnehwafgknerslkyierclqdflgfgvlgpegqlvswivmeqscelrmgytvpkyrhqgnmlqigyhlekylsqkeipfyfhva
dnnekslqalnnlgfkicpcgwhqwkctpkkyc
SEQ ID NO: 6 - GLYATL2-Nucleotide Sequence
atgcttgtgcttcataactctcagaagctgcagattctgtataaatccttagaaaagagcatccctgaatccataaaggtatatggcgccattttc
aacataaaagataaaaaccctttcaacatggaggtgctggtagatgcctggccagattaccagatcgtcattacccggcctcagaaacagga
gatgaaagatgaccaggatcattataccaacacttaccacatcttcaccaaagctcctgacaaattagaggaagtcctgtcatactccaatgta
atcagctgggagcaaactttgcagatccaaggttgccaagagggcttggatgaagcaataagaaaggttgcaacttcaaaatcagtgcagg
tagattacatgaaaaccatcctctttataccggaattaccaaagaaacacaagacctcaagtaatgacaagatggagttatttgaagtggatga
tgataacaaggaaggaaacttttcaaacatgttcttagatgcttcacatgcaggtcttgtgaatgaacactgggcctttgggaaaaatgagagg
agcttgaaatatattgaacgctgcctccaggattttctaggatttggtgtgctgggtccagagggccagcttgtctcttggattgtgatggaaca
gtcctgtgagttgagaatgggttatactgtccccaaatacagacaccaaggcaacatgttgcaaattggttatcatcttgaaaagtatctttctca
gaaagaaatcccattttatttccatgtggcagataataatgagaaaagcctacaggcactgaacaatttggggtttaagatttgtccttgtggct
ggcatcagtggaaatgcacccccaagaaatattgttga
SEQ ID NO: 7 - GLYATL3-Protein Sequence
MLVLNCSTKLLILEKMLKSCFPESLKVYGAVMNINRGNPFQKEVVLDSWPDFKAVITRR
QREAETDNLDHYTNAYAVFYKDVRAYRQLLEECDVFNWDQVFQIQGLQSELYDVSKA
VANSKQLNIKLTSFKAVHFSPVSSLPDTSFLKGPSPRLTYLSVANADLLNRTWSRGGNEQ
CLRYIANLISCFPSVCVRDEKGNPVSWSITDQFATMCHGYTLPEHRRKGYSRLVALTLA
RKLQSRGFPSQGNVLDDNTASISLLKSLHAEFLPCRFHRLILTPATFSGLPHL
SEQ ID NO: 8 - GLYATL3-Nucleotide Sequence
AAGAATAAACTTACCATTTATATAAAAGGGCTACTGGACTGATACACAGCTGAAAA
CCCTCAGTTCTGGACTGAACTCCCAGCAGGTGTGGAGTTGCAAGAGCTCTGGAAAA
GATGTTGGTGCTAAACTGTTCTACCAAATTACTGATACTGGAGAAAATGTTGAAGAG
TTGCTTTCCTGAATCACTCAAGGTTTACGGAGCGGTGATGAACATAAATCGTGGGAA
CCCCTTTCAAAAGGAAGTGGTGTTGGATTCATGGCCGGATTTCAAAGCTGTTATCAC
CCGACGACAAAGAGAGGCTGAGACAGATAACCTTGATCATTATACTAATGCCTATG
CTGTGTTCTACAAGGATGTCAGGGCTTATCGACAGCTATTGGAAGAATGTGATGTTT
TTAACTGGGACCAAGTTTTTCAAATACAAGGGCTGCAGAGTGAGTTATATGATGTTT
CCAAAGCGGTTGCCAATTCAAAGCAGTTGAATATAAAGCTAACTTCCTTCAAGGCTG
TTCATTTTTCTCCTGTTTCATCTCTGCCAGATACCAGTTTCCTCAAGGGGCCTTCCCC
ACGACTAACCTACCTGAGTGTTGCCAATGCGGATCTACTCAACCGGACTTGGTCCCG
GGGAGGCAATGAACAATGTCTCCGGTACATCGCCAACCTCATCTCCTGCTTCCCTAG
TGTGTGTGTCCGGGATGAGAAGGGAAACCCGGTCTCCTGGTCCATCACAGACCAGTT
TGCCACCATGTGCCATGGCTACACCCTGCCAGAACATCGCAGGAAAGGTTACAGCC
GGCTGGTGGCCCTCACGCTGGCCAGGAAGTTGCAAAGCCGGGGATTCCCCTCTCAG
GGGAACGTCCTGGATGACAACACGGCGTCTATAAGCCTCCTGAAGAGTCTCCATGCT
GAGTTCTTGCCTTGTCGCTTCCACAGGCTTATTCTCACCCCTGCGACTTTCTCTGGCC
TGCCTCACCTCTAGCCCAGTAAAAAACTGCAGTGGTTTTATTACTTTCCCTGAGCATA
CACACACTCTTGGCTGCCAACGAGGGGAGAGTTAAAATGGGAATCAGGGGACTCTT
GAGTTGTTGGAAAGGGTCTGGAGAATATATACAGGATCCACTTGAGAAGCCTTAATT
TTTCGTATCTCAGGTTTCTCCAGTAAATAGCTGTGGGGGTGAAGAGTAGCTGTGGCT
GAAGACTGAGGACGATTGTCCTCCTGTAGGATCCACTGTAGGAGAATAGGTTCTAA
AGCCAGCAGTTTTAGTGTACTAGGAGAAATTACTGCATGAGAACAAATGATTTAAC
AGAGGACCACGTGGCTACTGCTTTTTGATTGCTGCTTGGACCTCTGCTCTGTATTCTT
AAAGCCACACCGCTTCCCTACTGCCATCATATTCCCCTGTCCCCACTGCTATGTCTCA
TCAACCTCTGTTCCTAACACCTCTGCCACCAAGTTCTCTGTAGAGTAACCTCCTTTTT
CCCCTTTAATTACTTGCTCTTTACTTCTGCCTAGGACTCTAGCCTATAGTTCACTGCC
CTGGGAATGTTCAAATATAGTGGTTCTTACATTTTAGTGTTTATCAGAATCACCCAG
AGGGCAGGTTGCAACACACATCACTAGGCCTCTCCTTCTACGAGGTAGGGCCCAAA
ATTTGCATTTCTAACAGCTTCCCACTGCTTATTTGCCTTGGATGAATGACAATATGGG
CATTTTGATGCTATAAACAAATGCTGTCACCATAGAACTAGACTTTACCTATAACCT
ATTTCAGCCCCCTTATTTATAGTCTACTTTCCCATATAAAACTAAGATTTATATATAG
GGGTGTTTGGGGGTATGCAAATGAATATATAACATATATGCATACACATATATATAC
ATTCTCTTCATTTCTTTTATATGTATAGGTATATACTCATAGAATTTTGATAAGATAA
TAAATTTTAACCCTTTGATTACATATGAAAAATTTGAGGACCAGAGAAAATAAATGA
CTTTTTCAAGATTATATTCTTTATAATCAGTACTGGAGGCAAAGCCAGAATGCTGCC
ATTTTAATTCCAATCTGTTATTTTCACTAAATCATGTATCCTTTTTTATAATGAAAATT
AAAATGCTTACATAATTA
SEQ ID NO: 9 - Conserved motif
P(A/E)S(L/I)KVYG(T/A/S)(V/I)(F/M/Y)(H/N)I(N/K)(H/R/D)(G/K)NPF
SEQ ID NO: 10 - Conserved motif
D(D/N)(L/Q/M)D(H/S)YTN(T/A/V)Y
SEQ ID NO: 11 - Conserved motif
W(K/D/E)Q(H/V/T/R)(L/F)QIQ
SEQ ID NO: 12 - Conserved motif
L(V/L)N(K/R/E/D)(F/T/H/N)W(H/S/A/K)(F/R)G(G/K)NE
SEQ ID NO: 13 - Conserved motif
(G/D)(P/E)(E/K)G(T/N/Q/V)(P/L)V(C/S)W
SEQ ID NO: 14 - Codon optimized GLYAT
Atgatgctgccgctgcagggcgcacagatgctgcaaatgctggagaagtccctgcgtaagagcttgccggcttccctgaaagtttacggta
ccgtgttccacattaatcacggcaacccatttaacctgaaagccgtggttgacaagtggcctgactttaacactgtggttgtgtgcccgcaaga
gcaagacatgaccgacgatctggatcattatacgaatacgtatcagatctatagcaaagacccgcaaaattgccaggaatttctgggtagcc
cggagttgatcaattggaaacagcatctgcagattcaaagcagccaaccgagcttgaacgaagcgatccagaacctggcagcgattaagt
cgttcaaggtcaagcagacccaacgcattttgtacatggctgccgaaaccgcgaaagaactgacgccgttcctgttgaaaagcaagatcct
gtccccgaatggtggcaagccgaaagcgatcaatcaagaaatgttcaaactgagcagcatggatgtcacccacgcgcacctggtcaacaa
attctggcacttcggcggcaacgagcgtagccaacgttttatcgagcgctgtattcagacgtttccgacctgttgtctgctgggtcctgagggt
actccggtgtgctgggatctgatggatcagaccggtgagatgcgtatggccggtaccctgccagagtatcgcctgcacggcctggtcacgt
acgttatctacagccatgcgcagaaactgggtaagctgggtttcccggtgtactctcatgtcgactacagcaatgaagcaatgcaaaagatg
agctataccctgcagcacgttccgattccgcgttcttggaatcagtggaactgcgttccgctgtaa
SEQ ID NO: 15 - Codon optimized GLYATL-2
Atgctggtgctgcataattcgcaaaagctgcaaatcctgtacaaaagcctggagaagtccattccggagagcattaaagtgtatggtgcgat
ctttaacattaaggacaaaaaccctttcaacatggaagttctggttgacgcgtggccggattatcagatcgttattacccgtccacagaagcaa
gagatgaaagacgatcaagatcactacacgaatacctaccacatctttacgaaggctccggacaagctggaagaagtgttgagctattctaa
cgttatcagctgggagcaaacgctgcagattcagggttgtcaagagggcctggacgaagccatccgcaaagtcgcgaccagcaaaagcg
tccaagttgattacatgaaaaccatcctgttcatcccggaattgccgaagaaacataagacttccagcaacgataagatggaactgttcgagg
tcgatgacgacaataaggaaggcaactttagcaacatgtttttggatgcatctcatgccggtctggtgaacgagcactgggcgttcggcaaa
aatgaacgtagcctgaaatacattgagcgttgcctgcaggacttcctgggctttggtgtcctgggtccggaaggtcaactggtgagctggatt
gtgatggagcagagctgcgagttgcgtatgggctataccgtcccgaagtaccgccaccagggtaatatgctgcagatcggttatcatctgga
gaaatatctgagccagaaagaaattccgttttacttccacgttgcggacaataatgagaaaagcctgcaagcactgaacaatctgggtttcaa
gatttgcccgtgtggctggcaccagtggaaatgtaccccgaagaagtactgctaa
SEQ ID NO: 16 - Pspac promoter
Tacacagcccagtccagactattcggcactgaaattatgggtgaagtggtcaagacctcactaggcaccttaaaaatagcgcaccctgaag
aagatttatttgaggtagcccttgcctacctagcttccaagaaagatatcctaacagcacaagagcggaaagatgttttgttctacatccagaa
caacctctgctaaaattcctgaaaaattttgcaaaaagttgttgactttatctacaaggtgtggcataatgtgtggaattgtgagcggataacaat
t
SEQ ID NO: 17 - Ribosome Binding Sequence
Aaagcaaggaggagcagacgt
SEQ ID NO: 18 - Terminator Sequence
Agccgccccgcagggcgctccgcaggccgcttccggaccactccggaagcggccgtgcggtcgga
SEQ ID NO: 19 - Codon Optimized GLYAT Expression Construct
ggatcctacacagcccagtccagactattcggcactgaaattatgggtgaagtggtcaagacctcactaggcaccttaaaaatagcgcaccc
tgaagaagatttatttgaggtagcccttgcctacctagcttccaagaaagatatcctaacagcacaagagcggaaagatgttttgttctacatcc
agaacaacctctgctaaaattcctgaaaaattttgcaaaaagttgttgactttatctacaaggtgtggcataatgtgtggaattgtgagcggataa
caattaaagcaaggaggagcagacgtatgatgttaccattgcaaggtgcccagatgctgcagatgctggagaaatccttgaggaagagcct
cccagcatccttaaaggtttatggaactgtctttcacataaaccacggaaatccattcaatctgaaggctgtggtggacaagtggcctgatttta
atacagtggttgtctgccctcaggagcaggatatgacagatgaccttgatcactataccaatacttaccaaatctactccaaagatccccaaaa
ctgtcaggaattccttggatcaccagaactcatcaactggaaacagcatttacagattcaaagttcacagcctagcctgaatgaggctataca
aaatcttgcagccattaagtccttcaaagtcaaacaaacacaacgcattctctatatggcagctgaaacagccaaggaactgactcctttcctg
ctgaaatcaaagattttatctcccaatggtggcaaacccaaggccatcaaccaagagatgtttaaactctcatctatggatgttacccatgctca
cttggtgaataaattctggcattttggtggtaatgagaggagccagagattcattgagcgctgcattcagacctttcccacctgctgtctcctgg
ggcctgaggggacccctgtgtgctgggatctaatggaccagactggagagatgagaatggcaggcaccttgccggaataccggctccac
ggccttgtgacgtatgtcatctattcccacgcccagaaattgggcaaacttgggtttcctgtctattctcatgtagactacagcaatgaagctatg
caaaaaatgagttacacactgcaacatgttcccattcccagaagctggaaccagtggaactgtgtacctctgtgaagccgccccgcagggc
gctccgcaggccgcttccggaccactccggaagcggccgtgcggtcggaaagctttctaga
SEQ ID NO: 20 - Codon Optimized GLYAT Expression Construct
ggatcctacacagcccagtccagactattcggcactgaaattatgggtgaagtggtcaagacctcactaggcaccttaaaaatagcgcaccc
tgaagaagatttatttgaggtagcccttgcctacctagcttccaagaaagatatcctaacagcacaagagcggaaagatgttttgttctacatcc
agaacaacctctgctaaaattcctgaaaaattttgcaaaaagttgttgactttatctacaaggtgtggcataatgtgtggaattgtgagcggataa
caattaaagcaaggaggagcagacgtggatcctacacagcccagtccagactattcggcactgaaattatgggtgaagtggtcaagacctc
actaggcaccttaaaaatagcgcaccctgaagaagatttatttgaggtagcccttgcctacctagcttccaagaaagatatcctaacagcaca
agagcggaaagatgttttgttctacatccagaacaacctctgctaaaattcctgaaaaattttgcaaaaagttgttgactttatctacaaggtgtg
gcataatgtgtggaattgtgagcggataacaattaaagcaaggaggagcagacgtatgatgctgccgctgcagggcgcacagatgctgca
aatgctggagaagtccctgcgtaagagcttgccggatccctgaaagtttacggtaccgtgttccacattaatcacggcaacccatttaacctg
aaagccgtggttgacaagtggcctgactttaacactgtggttgtgtgcccgcaagagcaagacatgaccgacgatctggatcattatacgaa
tacgtatcagatctatagcaaagacccgcaaaattgccaggaatttctgggtagcccggagttgatcaattggaaacagcatctgcagattca
aagcagccaaccgagcttgaacgaagcgatccagaacctggcagcgattaagtcgttcaaggtcaagcagacccaacgcattttgtacatg
gctgccgaaaccgcgaaagaactgacgccgttcctgttgaaaagcaagatcctgtccccgaatggtggcaagccgaaagcgatcaatcaa
gaaatgttcaaactgagcagcatggatgtcacccacgcgcacctggtcaacaaattctggcacttcggcggcaacgagcgtagccaacgtt
ttatcgagcgctgtattcagacgtttccgacctgttgtctgctgggtcctgagggtactccggtgtgctgggatctgatggatcagaccggtga
gatgcgtatggccggtaccctgccagagtatcgcctgcacggcctggtcacgtacgttatctacagccatgcgcagaaactgggtaagctg
ggtttcccggtgtactctcatgtcgactacagcaatgaagcaatgcaaaagatgagctataccctgcagcacgttccgattccgcgttcttgga
atcagtggaactgcgttccgctgtaaagccgccccgcagggcgctccgcaggccgcttccggaccactccggaagcggccgtgcggtc
ggaaagctttctagaagccgccccgcagggcgctccgcaggccgcttccggaccactccggaagcggccgtgcggtcggaaagctttct
aga
SEQ ID NO: 21 - GLYATL2 Expression Construct
ggatcctacacagcccagtccagactattcggcactgaaattatgggtgaagtggtcaagacctcactaggcaccttaaaaatagcgcaccc
tgaagaagatttatttgaggtagcccttgcctacctagcttccaagaaagatatcctaacagcacaagagcggaaagatgttttgttctacatcc
agaacaacctctgctaaaattcctgaaaaattttgcaaaaagttgttgactttatctacaaggtgtggcataatgtgtggaattgtgagcggataa
caattaaagcaaggaggagcagacgtatgcttgtgcttcataactctcagaagctgcagattctgtataaatccttagaaaagagcatccctg
aatccataaaggtatatggcgccattttcaacataaaagataaaaaccctttcaacatggaggtgctggtagatgcctggccagattaccagat
cgtcattacccggcctcagaaacaggagatgaaagatgaccaggatcattataccaacacttaccacatcttcaccaaagctcctgacaaatt
agaggaagtcctgtcatactccaatgtaatcagctgggagcaaactttgcagatccaaggttgccaagagggcttggatgaagcaataaga
aaggttgcaacttcaaaatcagtgcaggtagattacatgaaaaccatcctctttataccggaattaccaaagaaacacaagacctcaagtaat
gacaagatggagttatttgaagtggatgatgataacaaggaaggaaacttttcaaacatgttcttagatgcttcacatgcaggtcttgtgaatga
acactgggcctttgggaaaaatgagaggagcttgaaatatattgaacgctgcctccaggattttctaggatttggtgtgctgggtccagaggg
ccagcttgtctcttggattgtgatggaacagtcctgtgagttgagaatgggttatactgtccccaaatacagacaccaaggcaacatgttgcaa
attggttatcatcttgaaaagtatctttctcagaaagaaatcccattttatttccatgtggcagataataatgagaaaagcctacaggcactgaac
aatttggggtttaagatttgtccttgtggctggcatcagtggaaatgcacccccaagaaatattgttgaagccgccccgcagggcgctccgca
ggccgcttccggaccactccggaagcggccgtgcggtcggaaagctttctaga
SEQ ID NO: 22 - Codon Optimized GLYATL2 Expression Construct
ggatcctacacagcccagtccagactattcggcactgaaattatgggtgaagtggtcaagacctcactaggcaccttaaaaatagcgcaccc
tgaagaagatttatttgaggtagcccttgcctacctagcttccaagaaagatatcctaacagcacaagagcggaaagatgttttgttctacatcc
agaacaacctctgctaaaattcctgaaaaattttgcaaaaagttgttgactttatctacaaggtgtggcataatgtgtggaattgtgagcggataa
caattaaagcaaggaggagcagacgtggatcctacacagcccagtccagactattcggcactgaaattatgggtgaagtggtcaagacctc
actaggcaccttaaaaatagcgcaccctgaagaagatttatttgaggtagcccttgcctacctagcttccaagaaagatatcctaacagcaca
agagcggaaagatgttttgttctacatccagaacaacctctgctaaaattcctgaaaaattttgcaaaaagttgttgactttatctacaaggtgtg
gcataatgtgtggaattgtgagcggataacaattaaagcaaggaggagcagacgtatgctggtgctgcataattcgcaaaagctgcaaatcc
tgtacaaaagcctggagaagtccattccggagagcattaaagtgtatggtgcgatctttaacattaaggacaaaaaccctttcaacatggaag
ttctggttgacgcgtggccggattatcagatcgttattacccgtccacagaagcaagagatgaaagacgatcaagatcactacacgaatacct
accacatctttacgaaggctccggacaagctggaagaagtgttgagctattctaacgttatcagctgggagcaaacgctgcagattcagggt
tgtcaagagggcctggacgaagccatccgcaaagtcgcgaccagcaaaagcgtccaagttgattacatgaaaaccatcctgttcatcccgg
aattgccgaagaaacataagacttccagcaacgataagatggaactgttcgaggtcgatgacgacaataaggaaggcaactttagcaacat
gtttttggatgcatctcatgccggtctggtgaacgagcactgggcgttcggcaaaaatgaacgtagcctgaaatacattgagcgttgcctgca
ggacttcctgggctttggtgtcctgggtccggaaggtcaactggtgagctggattgtgatggagcagagctgcgagttgcgtatgggctata
ccgtcccgaagtaccgccaccagggtaatatgctgcagatcggttatcatctggagaaatatctgagccagaaagaaattccgttttacttcc
acgttgcggacaataatgagaaaagcctgcaagcactgaacaatctgggtttcaagatttgcccgtgtggctggcaccagtggaaatgtacc
ccgaagaagtactgctaaagccgccccgcagggcgctccgcaggccgcttccggaccactccggaagcggccgtgcggtcggaaagc
tttctagaagccgccccgcagggcgctccgcaggccgcttccggaccactccggaagcggccgtgcggtcggaaagctttctaga
SEQ ID NO: 23 - Glyat Forward Primer
TTCTGTTTCTGCTTCGGTATGT
SEQ ID NO: 24 - Glyat Reverse Primer
GAGGCTTACTTGTCTGCTTTCT
SEQ ID NO: 25 - GlyatL2 Forward Primer
TTCTGTTTCTGCTTCGGTATGT
SEQ ID NO: 26 - GlyatL2 Reverse Primer
GAGGCTTACTTGTCTGCTTTCT
SEQ ID NO: 27 -
MGSSHHHHHHSSGLVPRGSHGMLPLQGAQMLQMLEKSLRKSLPASLKVYGTVFHINHG
NPFNLKAVVDKWPDFNTVVVCPQEQDMTDDLDHYTNTYQIYSKDPQNCQEFLGSPELI
NWKQHLQIQSSQPSLNEAIQNLAAIKSFKVKQTQRILYMAAETAKELTPFLLKSKILSPN
GGKPKAINQEMFKLSSMDVTHAHLVNKFWHFGGNERSQRFIERCIQTFPTCCLLGPEGT
PVCWDLMDQTGEMRMAGTLPEYRLHGLVTYVIYSHAQKLGKLGFPVYSHVDYSNEA
MQKMSYTLQHVPIPRSWNQWNCVPL
SEQ ID NO: 28 -
ATGGGCAGCAGCCATCATCATCATCATCACAGCAGCGGCCTGGTGCCGCGCGGCAG
CCACGGCATGTTACCATTGCAAGGTGCCCAGATGCTGCAGATGCTGGAGAAATCCTT
GAGGAAGAGCCTCCCAGCATCCTTAAAGGTTTATGGAACTGTCTTTCACATAAACCA
CGGAAATCCATTCAATCTGAAGGCTGTGGTGGACAAGTGGCCTGATTTTAATACAGT
GGTTGTCTGCCCTCAGGAGCAGGATATGACAGATGACCTTGATCACTATACCAATAC
TTACCAAATCTACTCCAAAGATCCCCAAAACTGTCAGGAATTCCTTGGATCACCAGA
ACTCATCAACTGGAAACAGCATTTACAGATTCAAAGTTCACAGCCTAGCCTGAATGA
GGCTATACAAAATCTTGCAGCCATTAAGTCCTTCAAAGTCAAACAAACACAACGCAT
TCTCTATATGGCAGCTGAAACAGCCAAGGAACTGACTCCTTTCCTGCTGAAATCAAA
GATTTTATCTCCCAATGGTGGCAAACCCAAGGCCATCAACCAAGAGATGTTTAAACT
CTCATCTATGGATGTTACCCATGCTCACTTGGTGAATAAATTCTGGCATTTTGGTGGT
AATGAGAGGAGCCAGAGATTCATTGAGCGCTGCATTCAGACCTTTCCCACCTGCTGT
CTCCTGGGGCCTGAGGGGACCCCTGTGTGCTGGGATCTAATGGACCAGACTGGAGA
GATGAGAATGGCAGGCACCTTGCCGGAATACCGGCTCCACGGCCTTGTGACGTATGT
CATCTATTCCCACGCCCAGAAATTGGGCAAACTTGGGTTTCCTGTCTATTCTCATGTA
GACTACAGCAATGAAGCTATGCAAAAAATGAGTTACACACTGCAACATGTTCCCATT
CCCAGAAGCTGGAACCAGTGGAACTGTGTACCTCTGTGA
SEQ ID NO: 29 -
MGSSHHHHHHSSGLVPRGSHGMLVLHNSQKLQILYKSLEKSIPESIKVYGAIFNIKDKNP
FNMEVLVDAWPDYQIVITRPQKQEMKDDQDHYTNTYHIFTKAPDKLEEVLSYSNVISW
EQTLQIQGCQEGLDEAIRKVATSKSVQVDYMKTILFIPELPKKHKTSSNDKMELFEVDDD
NKEGNFSNMFLDASHAGLVNEHWAFGKNERSLKYIERCLQDFLGFGVLGPEGQLVSWI
VMEQSCELRMGYTVPKYRHQGNMLQIGYHLEKYLSQKEIPFYFHVADNNEKSLQALNN
LGFKICPCGWHQWKCTPKKYC
SEQ ID NO: 30 -
ATGGGCAGCAGCCATCATCATCATCATCACAGCAGCGGCCTGGTGCCGCGCGGCAG
CCACGGCATGCTTGTGCTTCATAACTCTCAGAAGCTGCAGATTCTGTATAAATCCTT
AGAAAAGAGCATCCCTGAATCCATAAAGGTATATGGCGCCATTTTCAACATAAAAG
ATAAAAACCCTTTCAACATGGAGGTGCTGGTAGATGCCTGGCCAGATTACCAGATCG
TCATTACCCGGCCTCAGAAACAGGAGATGAAAGATGACCAGGATCATTATACCAAC
ACTTACCACATCTTCACCAAAGCTCCTGACAAATTAGAGGAAGTCCTGTCATACTCC
AATGTAATCAGCTGGGAGCAAACTTTGCAGATCCAAGGTTGCCAAGAGGGCTTGGA
TGAAGCAATAAGAAAGGTTGCAACTTCAAAATCAGTGCAGGTAGATTACATGAAAA
CCATCCTCTTTATACCGGAATTACCAAAGAAACACAAGACCTCAAGTAATGACAAG
ATGGAGTTATTTGAAGTGGATGATGATAACAAGGAAGGAAACTTTTCAAACATGTTC
TTAGATGCTTCACATGCAGGTCTTGTGAATGAACACTGGGCCTTTGGGAAAAATGAG
AGGAGCTTGAAATATATTGAACGCTGCCTCCAGGATTTTCTAGGATTTGGTGTGCTG
GGTCCAGAGGGCCAGCTTGTCTCTTGGATTGTGATGGAACAGTCCTGTGAGTTGAGA
ATGGGTTATACTGTCCCCAAATACAGACACCAAGGCAACATGTTGCAAATTGGTTAT
CATCTTGAAAAGTATCTTTCTCAGAAAGAAATCCCATTTTATTTCCATGTGGCAGAT
AATAATGAGAAAAGCCTACAGGCACTGAACAATTTGGGGTTTAAGATTTGTCCTTGT
GGCTGGCATCAGTGGAAATGCACCCCCAAGAAATATTGTTGA
SEQ ID NO: 31 - pHis-GLYAT-pETDuet-1
cctcgagtctggtaaagaaaccgctgctgcgaaatttgaacgccagcacatggactcgtctactagcgcagcttaattaacctaggctgctg
ccaccgctgagcaataactagcataaccccttggggcctctaaacgggtcttgaggggttttttgctgaaaggaggaactatatccggattgg
cgaatgggacgcgccctgtagcggcgcattaagcgcggcgggtgtggtggttacgcgcagcgtgaccgctacacttgccagcgccctag
cgcccgctcctttcgctttcttcccttcctttctcgccacgttcgccggctttccccgtcaagctctaaatcgggggctccctttagggttccgatt
tagtgctttacggcacctcgaccccaaaaaacttgattagggtgatggttcacgtagtgggccatcgccctgatagacggtttttcgccctttg
acgttggagtccacgttctttaatagtggactcttgttccaaactggaacaacactcaaccctatctcggtctattcttttgatttataagggattttg
ccgatttcggcctattggttaaaaaatgagctgatttaacaaaaatttaacgcgaattttaacaaaatattaacgtttacaatttctggcggcacga
tggcatgagattatcaaaaaggatcttcacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaacca
gccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaac
caagtcattctgagaatagtgtatgcggcgaccgagttgctcttgcccggcgtcaatacgggataataccgcgccacatagcagaactttaa
aagtgctcatcattggaaaacgttcttcggggcgaaaactctcaaggatcttaccgctgttgagatccagttcgatgtaacccactcgtgcacc
caactgatcttcagcatcttttactttcaccagcgtttctgggtgagcaaaaacaggaaggcaaaatgccgcaaaaaagggaataagggcga
cacggaaatgttgaatactcatactcttcctttttcaatcatgattgaagcatttatcagggttattgtctcatgagcggatacatatttgaatgtattt
agaaaaataaacaaataggtcatgaccaaaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagcggtggtttgtttgccggatcaagagctacc
aactctttttccgaaggtaactggcttcagcagagcgcagataccaaatactgtccttctagtgtagccgtagttaggccaccacttcaagaac
tctgtagcaccgcctacatacctcgctctgctaatcctgttaccagtggctgctgccagtggcgataagtcgtgtcttaccgggttggactcaa
gacgatagttaccggataaggcgcagcggtcgggctgaacggggggttcgtgcacacagcccagcttggagcgaacgacctacaccga
actgagatacctacagcgtgagctatgagaaagcgccacgcttcccgaagggagaaaggcggacaggtatccggtaagcggcagggtc
ggaacaggagagcgcacgagggagcttccagggggaaacgcctggtatctttatagtcctgtcgggtttcgccacctctgacttgagcgtc
gatttttgtgatgctcgtcaggggggcggagctatggaaaaacgccagcaacgcggcctttttacggttcctggccttttgctggccttttgct
cacatgttctttcctgcgttatcccctgattctgtggataaccgtattaccgcctttgagtgagctgataccgctcgccgcagccgaacgaccg
agcgcagcgagtcagtgagcgaggaagcggaagagcgcctgatgcggtattttctccttacgcatctgtgcggtatttcacaccgcatatat
ggtgcactctcagtacaatctgctctgatgccgcatagttaagccagtatacactccgctatcgctacgtgactgggtcatggctgcgccccg
acacccgccaacacccgctgacgcgccctgacgggcttgtctgctcccggcatccgcttacagacaagctgtgaccgtctccgggagctg
catgtgtcagaggttttcaccgtcatcaccgaaacgcgcgaggcagctgcggtaaagctcatcagcgtggtcgtgaagcgattcacagatg
tctgcctgttcatccgcgtccagctcgttgagtttctccagaagcgttaatgtctggcttctgataaagcgggccatgttaagggcggttttttcct
gtttggtcactgatgcctccgtgtaagggggatttctgttcatgggggtaatgataccgatgaaacgagagaggatgctcacgatacgggtta
ctgatgatgaacatgcccggttactggangttgtgagggtaaacaactggcggtatggatgcggcgggaccagagaaaaatcactcagg
gtcaatgccagcgcttcgttaatacagatgtaggtgttccacagggtagccagcagcatcctgcgatgcagatccggaacataatggtgcag
ggcgctgacttccgcgtttccagactttacgaaacacggaaaccgaagaccattcatgttgttgctcaggtcgcagacgttttgcagcagcag
tcgcttcacgttcgctcgcgtatcggtgattcattctgctaaccagtaaggcaaccccgccagcctagccgggtcctcaacgacaggagcac
gatcatgctagtcatgccccgcgcccaccggaaggagctgactgggttgaaggctctcaagggcatcggtcgagatcccggtgcctaatg
agtgagctaacttacattaattgcgttgcgctcactgcccgctttccagtcgggaaacctgtcgtgccagctgcattaatgaatcggccaacgc
gcggggagaggcggtttgcgtattgggcgccagggtggtttttcttttcaccagtgagacgggcaacagctgattgcccttcaccgcctggc
cctgagagagttgcagcaagcggtccacgctggtttgccccagcaggcgaaaatcctgtttgatggtggttaacggcgggatataacatga
gctgtcttcggtatcgtcgtatcccactaccgagatgtccgcaccaacgcgcagcccggactcggtaatggcgcgcattgcgcccagcgcc
atctgatcgttggcaaccagcatcgcagtgggaacgatgccctcattcagcatttgcatggtttgttgaaaaccggacatggcactccagtcg
ccttcccgttccgctatcggctgaatttgattgcgagtgagatatttatgccagccagccagacgcagacgcgccgagacagaacttaatgg
gcccgctaacagcgcgatttgctggtgacccaatgcgaccagatgctccacgcccagtcgcgtaccgtcttcatgggagaaaataatactgt
tgatgggtgtctggtcagagacatcaagaaataacgccggaacattagtgcaggcagcttccacagcaatggcatcctggtcatccagcgg
atagttaatgatcagcccactgacgcgttgcgcgagaagattgtgcaccgccgctttacaggcttcgacgccgcttcgttctaccatcgacac
caccacgctggcacccagttgatcggcgcgagatttaatcgccgcgacaatttgcgacggcgcgtgcagggccagactggaggtggcaa
cgccaatcagcaacgactgtttgcccgccagttgttgtgccacgcggttgggaatgtaattcagctccgccatcgccgcttccactttttcccg
cgttttcgcagaaacgtggctggcctggttcaccacgcgggaaacggtctgataagagacaccggcatactctgcgacatcgtataacgtta
ctggtttcacattcaccaccctgaattgactctcttccgggcgctatcatgccataccgcgaaaggttttgcgccattcgatggtgtccgggatc
tcgacgctctcccttatgcgactcctgcattaggaagcagcccagtagtaggttgaggccgttgagcaccgccgccgcaaggaatggtgca
tgcaaggagatggcgcccaacagtcccccggccacggggcctgccaccatacccacgccgaaacaagcgctcatgagcccgaagtgg
cgagcccgatcttccccatcggtgatgtcggcgatataggcgccagcaaccgcacctgtggcgccggtgatgccggccacgatgcgtcc
ggcgtagaggatcgagatcgatctcgatcccgcgaaattaatacgactcactataggggaattgtgagcggataacaattcccctctagaaa
taattttgtttaactttaagaaggagatataccatgggcagcagccatcatcatcatcatcacagcagcggcctggtgccgcgcggcagcca
cggcatgttaccattgcaaggtgcccagatgctgcagatgctggagaaatccttgaggaagagcctcccagcatccttaaaggtttatggaa
ctgtctttcacataaaccacggaaatccattcaatctgaaggctgtggtggacaagtggcctgattttaatacagtggttgtctgccctcaggag
caggatatgacagatgaccttgatcactataccaatacttaccaaatctactccaaagatccccaaaactgtcaggaattccttggatcaccag
aactcatcaactggaaacagcatttacagattcaaagttcacagcctagcctgaatgaggctatacaaaatcttgcagccattaagtccttcaa
agtcaaacaaacacaacgcattctctatatggcagctgaaacagccaaggaactgactcctttcctgctgaaatcaaagattttatctcccaat
ggtggcaaacccaaggccatcaaccaagagatgtttaaactctcatctatggatgttacccatgctcacttggtgaataaattctggcattttgg
tggtaatgagaggagccagagattcattgagcgctgcattcagacctttcccacctgctgtctcctggggcctgaggggacccctgtgtgct
gggatctaatggaccagactggagagatgagaatggcaggcaccttgccggaataccggctccacggccttgtgacgtatgtcatctattc
ccacgcccagaaattgggcaaacttgggtttcctgtctattacatgtagactacagcaatgaagctatgcaaaaaatgagttacacactgcaa
catgttcccattcccagaagctggaaccagtggaactgtgtacctctgtgataatagggtac
SEQ ID NO: 32 - pHis-GLYATL2-pETDuet-1
cctcgagtctggtaaagaaaccgctgctgcgaaatttgaacgccagcacatggactcgtctactagcgcagcttaattaacctaggctgctg
ccaccgctgagcaataactagcataaccccttggggcctctaaacgggtcttgaggggttttttgctgaaaggaggaactatatccggattgg
cgaatgggacgcgccctgtagcggcgcattaagcgcggcgggtgtggtggttacgcgcagcgtgaccgctacacttgccagcgccctag
cgcccgctcctttcgctttcttcccttcctttctcgccacgttcgccggctttccccgtcaagctctaaatcgggggctccctttagggttccgatt
tagtgctttacggcacctcgaccccaaaaaacttgattagggtgatggttcacgtagtgggccatcgccctgatagacggtttttcgccctttg
acgttggagtccacgttctttaatagtggactcttgttccaaactggaacaacactcaaccctatctcggtctattcttttgatttataagggattttg
ccgatttcggcctattggttaaaaaatgagctgatttaacaaaaatttaacgcgaattttaacaaaatattaacgtttacaatttctggcggcacga
tggcatgagattatcaaaaaggatcttcacctagatccttttaaattaaaaatgaagttttaaatcaatctaaagtatatatgagtaaacttggtctg
acagttaccaatgcttaatcagtgaggcacctatctcagcgatctgtctatttcgttcatccatagttgcctgactccccgtcgtgtagataacta
cgatacgggagggcttaccatctggccccagtgctgcaatgataccgcgagacccacgctcaccggctccagatttatcagcaataaacca
gccagccggaagggccgagcgcagaagtggtcctgcaactttatccgcctccatccagtctattaattgttgccgggaagctagagtaagta
gttcgccagttaatagtttgcgcaacgttgttgccattgctacaggcatcgtggtgtcacgctcgtcgtttggtatggcttcattcagctccggtt
cccaacgatcaaggcgagttacatgatcccccatgttgtgcaaaaaagcggttagctccttcggtcctccgatcgttgtcagaagtaagttgg
ccgcagtgttatcactcatggttatggcagcactgcataattctcttactgtcatgccatccgtaagatgcttttctgtgactggtgagtactcaac
caagtcattctgagaatagtgtatgcggcgaccgagttgctcttgcccggcgtcaatacgggataataccgcgccacatagcagaactttaa
aagtgctcatcattggaaaacgttcttcggggcgaaaactctcaaggatcttaccgctgttgagatccagttcgatgtaacccactcgtgcacc
caactgatcttcagcatcttttactttcaccagcgtttctgggtgagcaaaaacaggaaggcaaaatgccgcaaaaaagggaataagggcga
cacggaaatgttgaatactcatactcttcctttttcaatcatgattgaagcatttatcagggttattgtctcatgagcggatacatatttgaatgtattt
agaaaaataaacaaataggtcatgaccaaaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagcggtggtttgtttgccggatcaagagctacc
aactctttttccgaaggtaactggcttcagcagagcgcagataccaaatactgtccttctagtgtagccgtagttaggccaccacttcaagaac
tctgtagcaccgcctacatacctcgctctgctaatcctgttaccagtggctgctgccagtggcgataagtcgtgtcttaccgggttggactcaa
gacgatagttaccggataaggcgcagcggtcgggctgaacggggggttcgtgcacacagcccagcttggagcgaacgacctacaccga
actgagatacctacagcgtgagctatgagaaagcgccacgcttcccgaagggagaaaggcggacaggtatccggtaagcggcagggtc
ggaacaggagagcgcacgagggagcttccagggggaaacgcctggtatctttatagtcctgtcgggtttcgccacctctgacttgagcgtc
gatttttgtgatgctcgtcaggggggcggagcctatggaaaaacgccagcaacgcggcctttttacggttcctggccttttgctggccttttgct
cacatgttctttcctgcgttatcccctgattctgtggataaccgtattaccgcctttgagtgagctgataccgctcgccgcagccgaacgaccg
agcgcagcgagtcagtgagcgaggaagcggaagagcgcctgatgcggtattttctccttacgcatctgtgcggtatttcacaccgcatatat
ggtgcactctcagtacaatctgctctgatgccgcatagttaagccagtatacactccgctatcgctacgtgactgggtcatggctgcgccccg
acacccgccaacacccgctgacgcgccctgacgggcttgtctgctcccggcatccgcttacagacaagctgtgaccgtctccgggagctg
catgtgtcagaggttttcaccgtcatcaccgaaacgcgcgaggcagctgcggtaaagctcatcagcgtggtcgtgaagcgattcacagatg
tctgcctgttcatccgcgtccagctcgttgagtttctccagaagcgttaatgtctggcttctgataaagcgggccatgttaagggcggttttttcct
gtttggtcactgatgcctccgtgtaagggggatttctgttcatgggggtaatgataccgatgaaacgagagaggatgctcacgatacgggtta
ctgatgatgaacatgcccggttactggaacgttgtgagggtaaacaactggcggtatggatgcggcgggaccagagaaaaatcactcagg
gtcaatgccagcgcttcgttaatacagatgtaggtgttccacagggtagccagcagcatcctgcgatgcagatccggaacataatggtgcag
ggcgctgacttccgcgtttccagactttacgaaacacggaaaccgaagaccattcatgttgttgctcaggtcgcagacgttttgcagcagcag
tcgcttcacgttcgctcgcgtatcggtgattcattctgctaaccagtaaggcaaccccgccagcctagccgggtcctcaacgacaggagcac
gatcatgctagtcatgccccgcgcccaccggaaggagctgactgggttgaaggctctcaagggcatcggtcgagatcccggtgcctaatg
agtgagctaacttacattaattgcgttgcgctcactgcccgctttccagtcgggaaacctgtcgtgccagctgcattaatgaatcggccaacgc
gcggggagaggcggtttgcgtattgggcgccagggtggtttttcttttcaccagtgagacgggcaacagctgattgcccttcaccgcctggc
cctgagagagttgcagcaagcggtccacgctggtttgccccagcaggcgaaaatcctgtttgatggtggttaacggcgggatataacatga
gctgtcttcggtatcgtcgtatcccactaccgagatgtccgcaccaacgcgcagcccggactcggtaatggcgcgcattgcgcccagcgcc
atctgatcgttggcaaccagcatcgcagtgggaacgatgccctcattcagcatttgcatggtttgttgaaaaccggacatggcactccagtcg
ccttcccgttccgctatcggctgaatttgattgcgagtgagatatttatgccagccagccagacgcagacgcgccgagacagaacttaatgg
gcccgctaacagcgcgatttgctggtgacccaatgcgaccagatgctccacgcccagtcgcgtaccgtcttcatgggagaaaataatactgt
tgatgggtgtctggtcagagacatcaagaaataacgccggaacattagtgcaggcagcttccacagcaatggcatcctggtcatccagcgg
atagttaatgatcagcccactgacgcgttgcgcgagaagattgtgcaccgccgctttacaggcttcgacgccgcttcgttctaccatcgacac
caccacgctggcacccagttgatcggcgcgagatttaatcgccgcgacaatttgcgacggcgcgtgcagggccagactggaggtggcaa
cgccaatcagcaacgactgtttgcccgccagttgttgtgccacgcggttgggaatgtaattcagctccgccatcgccgcttccactttttcccg
cgttttcgcagaaacgtggctggcctggttcaccacgcgggaaacggtctgataagagacaccggcatactctgcgacatcgtataacgtta
ctggtttcacattcaccaccctgaattgactctcttccgggcgctatcatgccataccgcgaaaggttttgcgccattcgatggtgtccgggatc
tcgacgctctcccttatgcgactcctgcattaggaagcagcccagtagtaggttgaggccgttgagcaccgccgccgcaaggaatggtgca
tgcaaggagatggcgcccaacagtcccccggccacggggcctgccaccatacccacgccgaaacaagcgctcatgagcccgaagtgg
cgagcccgatcttccccatcggtgatgtcggcgatataggcgccagcaaccgcacctgtggcgccggtgatgccggccacgatgcgtcc
ggcgtagaggatcgagatcgatctcgatcccgcgaaattaatacgactcactataggggaattgtgagcggataacaattcccctctagaaa
taattttgtttaactttaagaaggagatataccatgggcagcagccatcatcatcatcatcacagcagcggcctggtgccgcgcggcagcca
cggcatgcttgtgcttcataactctcagaagctgcagattctgtataaatccttagaaaagagcatccctgaatccataaaggtatatggcgcc
attttcaacataaaagataaaaaccctttcaacatggaggtgctggtagatgcctggccagattaccagatcgtcattacccggcctcagaaa
caggagatgaaagatgaccaggatcattataccaacacttaccacatcttcaccaaagctcctgacaaattagaggaagtcctgtcatactcc
aatgtaatcagctgggagcaaactttgcagatccaaggttgccaagagggcttggatgaagcaataagaaaggttgcaacttcaaaatcagt
gcaggtagattacatgaaaaccatcctctttataccggaattaccaaagaaacacaagacctcaagtaatgacaagatggagttatttgaagt
ggatgatgataacaaggaaggaaacttttcaaacatgttcttagatgcttcacatgcaggtcttgtgaatgaacactgggcctttgggaaaaat
gagaggagcttgaaatatattgaacgctgcctccaggattttctaggatttggtgtgctgggtccagagggccagcttgtctcttggattgtgat
ggaacagtcctgtgagttgagaatgggttatactgtccccaaatacagacaccaaggcaacatgttgcaaattggttatcatcttgaaaagtat
ctttctcagaaagaaatcccattttatttccatgtggcagataataatgagaaaagcctacaggcactgaacaatttggggtttaagatttgtcctt
gtggctggcatcagtggaaatgcacccccaagaaatattgttgataatagggtac

What is claimed is:

1. A metabolically-engineered microorganism capable of synthesizing an N-acylglycine biosurfactant, the microorganism comprising a Glycine N-Acyltransferase protein.

2. The metabolically-engineered microorganism of claim 1, the Glycine N-Acyltransferase protein selected from the group consisting of:
a. a polypeptide with at least 90% sequence identity to a GLYAT polypeptide of SEQ ID NO:1;
b. a polypeptide with at least 90% sequence identity to a GLYATL 1 polypeptide of SEQ ID:3;
c. a polypeptide with at least 90% sequence identity to a GLYATL 2 polypeptide of SEQ ID NO:5;
d. a polypeptide with at least 90% sequence identity to a GLYATL 3 polypeptide of SEQ ID NO:7;
e. a polypeptide comprising at least one of the polypeptide motifs of:
i. P(A/E)S(L/I)KVYG(T/A/S)(V/I)(F/M/Y)(H/N)I(N/K)(H/R/D)(G/K)NPF (SEQ ID NO: 9),
ii. D(D/N)(L/Q/M)D(H/S)YTN(T/A/V)Y (SEQ ID NO: 10),
iii. W(K/D/E)Q(H/V/T/R)(L/F)QIQ (SEQ ID NO: 11),
iv. L(V/L)N(K/R/E/D)(F/T/H/N)W(H/S/A/K)(F/R)G(G/K)NE (SEQ ID NO: 12), or
v. (G/D)(P/E)(E/K)G(T/N/Q/V)(P/L)V(C/S)W (SEQ ID NO: 13);
f. a variant polypeptide of SEQ ID NO: 1, said variant having Glycine N-Acyltransferase activity and a least 90% sequence identity with a sequence selected from SEQ ID NO: 1;
g. a variant polypeptide of SEQ ID NO:3, said variant having Glycine N-Acyltransferase activity and a least 90% sequence identity with a sequence selected from SEQ ID NO:3;
h. a variant polypeptide of SEQ ID NO:5, said variant having Glycine N-Acyltransferase activity and a least 90% sequence identity with a sequence selected from SEQ ID NO:5;
i. a variant polypeptide of SEQ ID NO:7, said variant having Glycine N-Acyltransferase activity and a least 90% sequence identity with a sequence selected from SEQ ID NO:7;
j. a polypeptide having Glycine N-Acyltransferase activity wherein said polypeptide is encoded by an isolated polynucleotide that hybridizes under stringent conditions with the sense or anti-sense strand of a polynucleotide sequence selected from SEQ ID NO:2, SEQ ID NO:4, SEQ ID NO:6, SEQ ID NO:8, SEQ ID NO:14, or SEQ ID NO:15; and,
k. a polypeptide that facilitates the conversion of acyl-coA and glycine into coA and N-acylglycine and the polypeptide is chosen from a Glycine N-Acyltransferase enzyme of class E.C. 2.3.1.13.

3. The metabolically-engineered microorganism of claim 1, wherein the Glycine N-Acyltransferase protein comprises a polypeptide with at least 90% sequence identity to a GLYAT of SEQ ID NO: 1.

4. The metabolically-engineered microorganism of claim 1, wherein the Glycine N-Acyltransferase protein comprises a polypeptide with at least 90% sequence identity to a GLYATL 1 of SEQ ID NO:3.

5. The metabolically-engineered microorganism of claim 1, wherein the Glycine N-Acyltransferase protein comprises a polypeptide with at least 90% sequence identity to a GLYATL 2 of SEQ ID NO:5.

6. The metabolically-engineered microorganism of claim 1, wherein the Glycine N-Acyltransferase protein comprises a polypeptide with at least 90% sequence identity to a GLYATL 3 of SEQ ID NO:7.

7. The metabolically-engineered microorganism of claim 1, wherein the microorganism is a gram (−) or a gram (+) bacteria.

8. The metabolically-engineered microorganism of claim 7, wherein the gram (+) bacteria is Bacillus subtilis.

9. The metabolically-engineered microorganism of claim 7, wherein the gram (−) bacteria Escherichia coli.

10. The metabolically-engineered microorganism of claim 1, wherein a polynucleotide encoding the Glycine N-Acyltransferase protein is expressed by a bacterial promoter.

11. The metabolically-engineered microorganism of claim 10, wherein the polynucleotide encoding the Glycine N-Acyltransferase protein is codon optimized for expression in the microorganism.

12. The metabolically-engineered microorganism of claim 11, wherein the codon optimized polynucleotide encoding the Glycine N-Acyltransferase protein is selected from the group consisting of SEQ ID NO: 14 and SEQ ID NO: 15.

13. The metabolically-engineered microorganism of claim 10, wherein the bacterial promoter comprises a PsPAC bacterial promoter.

14. The metabolically-engineered microorganism of claim 1, wherein a polynucleotide encoding the Glycine N-Acyltransferase protein is integrated within a genomic locus of the microorganism, or is integrated within an autonomously replicating plasmid.

15. The metabolically-engineered microorganism of claim 14, wherein the genomic locus comprises an amyE genomic locus.

16. The metabolically-engineered microorganism of claim 14, wherein the integration comprises a homologous recombination mediated integration.

17. The metabolically-engineered microorganism of claim 1, wherein the expression of the Glycine N-Acyltransferase protein results in the synthesis of N-acylglycine from medium chain length β-hydroxy fatty acids.

18. A method for producing N-acylglycine from a microorganism, the method comprising;
a. obtaining a microorganism comprising a polynucleotide encoding a Glycine N-Acyltransferase protein of claim 1;
b. culturing the microorganism to produce medium chain length β-hydroxy fatty acid;
c. expressing the Glycine N-Acyltransferase protein, wherein the expression of the Glycine N-Acyltransferase protein synthesizes N-acylglycine from the medium chain length β-hydroxy fatty acid; and,
d. purifying the N-acylglycine from the microorganism to produce the N-acylglycine.

19. A method for fermenting N-acylglycine within a microorganism, the method comprising;
a. obtaining a microorganism comprising a polynucleotide encoding a Glycine N-Acyltransferase protein of claim 1;
b. expressing the Glycine N-Acyltransferase protein; wherein the expression of the Glycine N-Acyltransferase protein synthesizes N-acylglycine from a medium chain length β-hydroxy fatty acid; and,
c. fermenting N-acylglycine within the microorganism.
